SEQUENCE LISTING (44 sequences)

SEQ ID NO: 1 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 1211 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIK

SEQ ID NO: 3 - protein, 1188 aa, Severe acute respiratory syndrome coronavirus 2
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQ

SEQ ID NO: 4 - protein, 1198 aa, Artificial Sequence
  Description of Artificial Sequence Synthetic polypeptide
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIK

SEQ ID NO: 5 - protein, 1198 aa, Artificial Sequence
  Description of Artificial Sequence Synthetic polypeptide
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPAGAGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIK

SEQ ID NO: 6 - protein, 1198 aa, Artificial Sequence
  Description of Artificial Sequence Synthetic polypeptide
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPAGAGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIK

SEQ ID NO: 7 - protein, 1198 aa, Artificial Sequence
  Description of Artificial Sequence Synthetic polypeptide
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPAGAGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIK

SEQ ID NO: 8 - protein, 1234 aa, Artificial Sequence
  Description of Artificial Sequence Synthetic polypeptide
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPAGAGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKGSGYIPEAPRDGQAYVRKDGEWVLLSTFLGHHHHHH

SEQ ID NO: 9 - protein, 1247 aa, Artificial Sequence
  Description of Artificial Sequence Synthetic polypeptide
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPAGAGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKGSGYIPEAPRDGQAYVRKDGEWVLLSTFLGHHHHHH

SEQ ID NO: 10 - protein, 1234 aa, Artificial Sequence
  Description of Artificial Sequence Synthetic polypeptide
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPAGAGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKGSGYIPEAPRDGQAYVRKDGEWVLLSTFLGHHHHHH

SEQ ID NO: 11 - protein, 1247 aa, Artificial Sequence
  Description of Artificial Sequence Synthetic polypeptide
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPAGAGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKGSGYIPEAPRDGQAYVRKDGEWVLLSTFLGHHHHHH

SEQ ID NO: 12 - protein, 1240 aa, Artificial Sequence
  Description of Artificial Sequence Synthetic polypeptide
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPAGAGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKGSGIKRMKQIEDKIEEIESKQKKIENEIARIKKIKGHHHHHH

SEQ ID NO: 13 - protein, 1253 aa, Artificial Sequence
  Description of Artificial Sequence Synthetic polypeptide
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPAGAGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKGSGIKRMKQIEDKIEEIESKQKKIENEIARIKKIKGHHHHHH

SEQ ID NO: 14 - protein, 13 aa, Severe acute respiratory syndrome coronavirus 2
MFVFLVLLPLVSS

SEQ ID NO: 15 - protein, 3 aa, Artificial Sequence
  Description of Artificial Sequence Synthetic peptide
GSG

SEQ ID NO: 16 - protein, 26 aa, Escherichia virus T4
YIPEAPRDGQAYVRKDGEWVLLSTFL

SEQ ID NO: 17 - protein, 32 aa, Saccharomyces cerevisiae
IKRMKQIEDKIEEIESKQKKIENEIARIKKIK

SEQ ID NO: 18 - protein, 30 aa, Artificial Sequence
  Description of Artificial Sequence Synthetic polypeptide
GSGYIPEAPRDGQAYVRKDGEWVLLSTFLG

SEQ ID NO: 19 - protein, 36 aa, Artificial Sequence
  Description of Artificial Sequence Synthetic polypeptide
GSGIKRMKQIEDKIEEIESKQKKIENEIARIKKIKG

SEQ ID NO: 20 - protein, 6 aa, Artificial Sequence
  Description of Artificial Sequence Synthetic 6xHis tag
HHHHHH

SEQ ID NO: 21 - DNA, 3819 bp, Severe acute respiratory syndrome coronavirus 2
atgtttgtcttcctggtcctgctgcctctggtctcgtctcagtgcgtgaacctgactactagaacccagctgcctcctgcctatactaactccttcacccgcggcgtgtactacccagacaaggtgttccgcagctccgtgctgcactccacccaggatctgttcctgcccttcttcagcaacgtgacctggttccacgccatccacgtgagcggcaccaatggcaccaagcggttcgacaatcccgtgctgccattcaacgatggcgtgtacttcgcctccaccgagaagagcaacatcatccgcggctggatcttcggcaccaccctggactccaagacccagagcctgctgatcgtgaacaatgccaccaacgtggtcatcaaggtgtgcgagttccagttctgcaatgatccattcctgggcgtgtactaccacaagaacaataagtcctggatggagagcgagttccgcgtgtacagctccgccaacaattgcaccttcgagtacgtgtcccagcccttcctgatggacctggagggcaagcagggcaatttcaagaacctgcgcgagttcgtgttcaagaatatcgatggctacttcaagatctactccaagcacacccccatcaacctggtgcgcgacctgccacagggcttcagcgccctggagccactggtggatctgccaatcggcatcaacatcaccaggttccagaccctgctggccctgcaccgcagctacctgaccccaggcgacagctccagcggatggaccgctggagctgctgcctactacgtgggctacctgcagccccgcaccttcctgctgaagtacaacgagaatggcaccatcaccgacgccgtggattgcgccctggatccactgtccgagacaaagtgcaccctgaagagcttcaccgtggagaagggcatctaccagacctccaatttccgcgtgcagccaaccgagagcatcgtgcgcttccccaatatcaccaacctgtgcccattcggcgaggtgttcaacgctaccaggttcgccagcgtgtacgcttggaatcgcaagcgcatctccaactgcgtggccgactacagcgtgctgtacaactccgccagcttctccaccttcaagtgctacggcgtgtcccccaccaagctgaatgatctgtgcttcaccaacgtgtacgccgatagcttcgtgatcaggggcgacgaggtgcgccagatcgctccaggacagaccggcaagatcgctgactacaattacaagctgcccgacgatttcaccggctgcgtgatcgcctggaactccaacaatctggatagcaaagtgggcggcaactacaattacctgtaccgcctgttccgcaagtccaatctgaagccattcgagcgcgacatctccaccgagatctaccaggctggaagcaccccatgcaatggagtggagggcttcaactgctacttccccctgcagagctacggcttccagccaaccaacggagtgggataccagccatacagggtggtggtgctgtccttcgagctgctgcacgctccagctaccgtgtgcggaccaaagaagagcaccaatctggtgaagaacaagtgcgtgaacttcaatttcaacggcctgaccggaaccggcgtgctgaccgagtccaacaagaagttcctgccattccagcagttcggaagggacatcgctgataccaccgacgccgtgcgcgacccacagaccctggagatcctggatatcaccccatgctccttcggcggcgtgagcgtgatcaccccaggaaccaataccagcaaccaggtggccgtgctgtaccaggacgtgaattgcaccgaggtgccagtggctatccacgctgatcagctgaccccaacctggcgcgtgtacagcaccggatccaacgtgttccagacccgcgccggatgcctgatcggagctgagcacgtgaacaattcctacgagtgcgacatcccaatcggagctggaatctgcgccagctaccagacccagaccaactccccaaggagggctcgcagcgtggccagccagtccatcatcgcctacaccatgtccctgggcgccgagaatagcgtggcctacagcaacaattccatcgccatcccaaccaacttcaccatctccgtgaccaccgagatcctgcccgtgtccatgaccaagaccagcgtggactgcaccatgtacatctgcggcgattccaccgagtgcagcaacctgctgctgcagtacggcagcttctgcacccagctgaatcgcgccctgaccggaatcgctgtggagcaggataagaacacccaggaggtgttcgcccaggtgaagcagatctacaagacccccccaatcaaggacttcggcggcttcaatttcagccagatcctgcccgatccaagcaagccctccaagcgcagcttcatcgaggacctgctgttcaacaaggtgaccctggccgatgccggcttcatcaagcagtacggcgattgcctgggcgacatcgctgcccgcgacctgatctgcgcccagaagttcaatggcctgaccgtgctgccaccactgctgaccgatgagatgatcgctcagtacacctccgccctgctggccggaaccatcaccagcggatggaccttcggcgctggagccgccctgcagatccccttcgccatgcagatggcctaccgcttcaacggcatcggcgtgacccagaatgtgctgtacgagaaccagaagctgatcgccaatcagttcaactccgccatcggcaagatccaggactccctgtccagcaccgccagcgccctgggcaagctgcaggatgtggtgaatcagaacgcccaggccctgaataccctggtgaagcagctgtccagcaacttcggcgccatctccagcgtgctgaatgatatcctgagccgcctggacaaggtggaggctgaggtgcagatcgataggctgatcaccggccgcctgcagtccctgcagacctacgtgacccagcagctgatcagggctgctgagatcagggccagcgccaatctggctgctaccaagatgtccgagtgcgtgctgggacagagcaagagggtggacttctgcggcaagggctaccacctgatgtccttcccacagagcgccccacacggagtggtgttcctgcacgtgacctacgtgccagcccaggagaagaacttcaccaccgctccagctatctgccacgatggcaaggctcacttcccacgcgagggcgtgttcgtgtccaacggcacccactggttcgtgacccagcgcaatttctacgagccccagatcatcaccaccgacaataccttcgtgagcggcaactgcgacgtggtcatcggaatcgtgaacaataccgtgtacgatcccctgcagccagagctggactccttcaaggaggagctggataagtacttcaagaatcacaccagccccgacgtggatctgggcgacatctccggcatcaatgccagcgtggtgaacatccagaaggagatcgaccgcctgaacgaggtggccaagaatctgaacgagtccctgatcgatctgcaggagctgggcaagtacgagcagtacatcaagtggccatggtacatctggctgggcttcatcgccggcctgatcgccatcgtgatggtgaccatcatgctgtgctgcatgacctcctgctgcagctgcctgaagggctgctgctcctgcggcagctgctgcaagttcgatgaggacgatagcgagcccgtgctgaagggcgtcaaactgcactataca

SEQ ID NO: 22 - DNA, 3741 bp, Artificial Sequence
  Description of Artificial Sequence Synthetic polynucleotide
atgtttgtcttcctggtcctgctgcctctggtctcgtctcagtgcgtgaacctgactactagaacccagctgcctcctgcctatactaactccttcacccgcggcgtgtactacccagacaaggtgttccgcagctccgtgctgcactccacccaggatctgttcctgcccttcttcagcaacgtgacctggttccacgccatccacgtgagcggcaccaatggcaccaagcggttcgacaatcccgtgctgccattcaacgatggcgtgtacttcgcctccaccgagaagagcaacatcatccgcggctggatcttcggcaccaccctggactccaagacccagagcctgctgatcgtgaacaatgccaccaacgtggtcatcaaggtgtgcgagttccagttctgcaatgatccattcctgggcgtgtactaccacaagaacaataagtcctggatggagagcgagttccgcgtgtacagctccgccaacaattgcaccttcgagtacgtgtcccagcccttcctgatggacctggagggcaagcagggcaatttcaagaacctgcgcgagttcgtgttcaagaatatcgatggctacttcaagatctactccaagcacacccccatcaacctggtgcgcgacctgccacagggcttcagcgccctggagccactggtggatctgccaatcggcatcaacatcaccaggttccagaccctgctggccctgcaccgcagctacctgaccccaggcgacagctccagcggatggaccgctggagctgctgcctactacgtgggctacctgcagccccgcaccttcctgctgaagtacaacgagaatggcaccatcaccgacgccgtggattgcgccctggatccactgtccgagacaaagtgcaccctgaagagcttcaccgtggagaagggcatctaccagacctccaatttccgcgtgcagccaaccgagagcatcgtgcgcttccccaatatcaccaacctgtgcccattcggcgaggtgttcaacgctaccaggttcgccagcgtgtacgcttggaatcgcaagcgcatctccaactgcgtggccgactacagcgtgctgtacaactccgccagcttctccaccttcaagtgctacggcgtgtcccccaccaagctgaatgatctgtgcttcaccaacgtgtacgccgatagcttcgtgatcaggggcgacgaggtgcgccagatcgctccaggacagaccggcaagatcgctgactacaattacaagctgcccgacgatttcaccggctgcgtgatcgcctggaactccaacaatctggatagcaaagtgggcggcaactacaattacctgtaccgcctgttccgcaagtccaatctgaagccattcgagcgcgacatctccaccgagatctaccaggctggaagcaccccatgcaatggagtggagggcttcaactgctacttccccctgcagagctacggcttccagccaaccaacggagtgggataccagccatacagggtggtggtgctgtccttcgagctgctgcacgctccagctaccgtgtgcggaccaaagaagagcaccaatctggtgaagaacaagtgcgtgaacttcaatttcaacggcctgaccggaaccggcgtgctgaccgagtccaacaagaagttcctgccattccagcagttcggaagggacatcgctgataccaccgacgccgtgcgcgacccacagaccctggagatcctggatatcaccccatgctccttcggcggcgtgagcgtgatcaccccaggaaccaataccagcaaccaggtggccgtgctgtaccagggcgtgaattgcaccgaggtgccagtggctatccacgctgatcagctgaccccaacctggcgcgtgtacagcaccggatccaacgtgttccagacccgcgccggatgcctgatcggagctgagcacgtgaacaattcctacgagtgcgacatcccaatcggagctggaatctgcgccagctaccagacccagaccaactccccagctggagctggcagcgtggccagccagtccatcatcgcctacaccatgtccctgggcgccgagaatagcgtggcctacagcaacaattccatcgccatcccaaccaacttcaccatctccgtgaccaccgagatcctgcccgtgtccatgaccaagaccagcgtggactgcaccatgtacatctgcggcgattccaccgagtgcagcaacctgctgctgcagtacggcagcttctgcacccagctgaatcgcgccctgaccggaatcgctgtggagcaggataagaacacccaggaggtgttcgcccaggtgaagcagatctacaagacccccccaatcaaggacttcggcggcttcaatttcagccagatcctgcccgatccaagcaagccctccaagcgcagcttcatcgaggacctgctgttcaacaaggtgaccctggccgatgccggcttcatcaagcagtacggcgattgcctgggcgacatcgctgcccgcgacctgatctgcgcccagaagttcaatggcctgaccgtgctgccaccactgctgaccgatgagatgatcgctcagtacacctccgccctgctggccggaaccatcaccagcggatggaccttcggcgctggagccgccctgcagatccccttcgccatgcagatggcctaccgcttcaacggcatcggcgtgacccagaatgtgctgtacgagaaccagaagctgatcgccaatcagttcaactccgccatcggcaagatccaggactccctgtccagcaccgccagcgccctgggcaagctgcaggatgtggtgaatcagaacgcccaggccctgaataccctggtgaagcagctgtccagcaacttcggcgccatctccagcgtgctgaatgatatcctgagccgcctggacccaccggaggctgaggtgcagatcgataggctgatcaccggccgcctgcagtccctgcagacctacgtgacccagcagctgatcagggctgctgagatcagggccagcgccaatctggctgctaccaagatgtccgagtgcgtgctgggacagagcaagagggtggacttctgcggcaagggctaccacctgatgtccttcccacagagcgccccacacggagtggtgttcctgcacgtgacctacgtgccagcccaggagaagaacttcaccaccgctccagctatctgccacgatggcaaggctcacttcccacgcgagggcgtgttcgtgtccaacggcacccactggttcgtgacccagcgcaatttctacgagccccagatcatcaccaccgacaataccttcgtgagcggcaactgcgacgtggtcatcggaatcgtgaacaataccgtgtacgatcccctgcagccagagctggactccttcaaggaggagctggataagtacttcaagaatcacaccagccccgacgtggatctgggcgacatctccggcatcaatgccagcgtggtgaacatccagaaggagatcgaccgcctgaacgaggtggccaagaatctgaacgagtccctgatcgatctgcaggagctgggcaagtacgagcagtacatcaagggatccggctacatccccgaggcccccagagacggccaggcctacgtgagaaaggacggcgagtgggtgctgctgagcaccttcctgggccaccaccaccaccaccac

SEQ ID NO: 23 - DNA, 3759 bp, Artificial Sequence
  Description of Artificial Sequence Synthetic polynucleotide
atgtttgtcttcctggtcctgctgcctctggtctcgtctcagtgcgtgaacctgactactagaacccagctgcctcctgcctatactaactccttcacccgcggcgtgtactacccagacaaggtgttccgcagctccgtgctgcactccacccaggatctgttcctgcccttcttcagcaacgtgacctggttccacgccatccacgtgagcggcaccaatggcaccaagcggttcgacaatcccgtgctgccattcaacgatggcgtgtacttcgcctccaccgagaagagcaacatcatccgcggctggatcttcggcaccaccctggactccaagacccagagcctgctgatcgtgaacaatgccaccaacgtggtcatcaaggtgtgcgagttccagttctgcaatgatccattcctgggcgtgtactaccacaagaacaataagtcctggatggagagcgagttccgcgtgtacagctccgccaacaattgcaccttcgagtacgtgtcccagcccttcctgatggacctggagggcaagcagggcaatttcaagaacctgcgcgagttcgtgttcaagaatatcgatggctacttcaagatctactccaagcacacccccatcaacctggtgcgcgacctgccacagggcttcagcgccctggagccactggtggatctgccaatcggcatcaacatcaccaggttccagaccctgctggccctgcaccgcagctacctgaccccaggcgacagctccagcggatggaccgctggagctgctgcctactacgtgggctacctgcagccccgcaccttcctgctgaagtacaacgagaatggcaccatcaccgacgccgtggattgcgccctggatccactgtccgagacaaagtgcaccctgaagagcttcaccgtggagaagggcatctaccagacctccaatttccgcgtgcagccaaccgagagcatcgtgcgcttccccaatatcaccaacctgtgcccattcggcgaggtgttcaacgctaccaggttcgccagcgtgtacgcttggaatcgcaagcgcatctccaactgcgtggccgactacagcgtgctgtacaactccgccagcttctccaccttcaagtgctacggcgtgtcccccaccaagctgaatgatctgtgcttcaccaacgtgtacgccgatagcttcgtgatcaggggcgacgaggtgcgccagatcgctccaggacagaccggcaagatcgctgactacaattacaagctgcccgacgatttcaccggctgcgtgatcgcctggaactccaacaatctggatagcaaagtgggcggcaactacaattacctgtaccgcctgttccgcaagtccaatctgaagccattcgagcgcgacatctccaccgagatctaccaggctggaagcaccccatgcaatggagtggagggcttcaactgctacttccccctgcagagctacggcttccagccaaccaacggagtgggataccagccatacagggtggtggtgctgtccttcgagctgctgcacgctccagctaccgtgtgcggaccaaagaagagcaccaatctggtgaagaacaagtgcgtgaacttcaatttcaacggcctgaccggaaccggcgtgctgaccgagtccaacaagaagttcctgccattccagcagttcggaagggacatcgctgataccaccgacgccgtgcgcgacccacagaccctggagatcctggatatcaccccatgctccttcggcggcgtgagcgtgatcaccccaggaaccaataccagcaaccaggtggccgtgctgtaccagggcgtgaattgcaccgaggtgccagtggctatccacgctgatcagctgaccccaacctggcgcgtgtacagcaccggatccaacgtgttccagacccgcgccggatgcctgatcggagctgagcacgtgaacaattcctacgagtgcgacatcccaatcggagctggaatctgcgccagctaccagacccagaccaactccccagctggagctggcagcgtggccagccagtccatcatcgcctacaccatgtccctgggcgccgagaatagcgtggcctacagcaacaattccatcgccatcccaaccaacttcaccatctccgtgaccaccgagatcctgcccgtgtccatgaccaagaccagcgtggactgcaccatgtacatctgcggcgattccaccgagtgcagcaacctgctgctgcagtacggcagcttctgcacccagctgaatcgcgccctgaccggaatcgctgtggagcaggataagaacacccaggaggtgttcgcccaggtgaagcagatctacaagacccccccaatcaaggacttcggcggcttcaatttcagccagatcctgcccgatccaagcaagccctccaagcgcagcttcatcgaggacctgctgttcaacaaggtgaccctggccgatgccggcttcatcaagcagtacggcgattgcctgggcgacatcgctgcccgcgacctgatctgcgcccagaagttcaatggcctgaccgtgctgccaccactgctgaccgatgagatgatcgctcagtacacctccgccctgctggccggaaccatcaccagcggatggaccttcggcgctggagccgccctgcagatccccttcgccatgcagatggcctaccgcttcaacggcatcggcgtgacccagaatgtgctgtacgagaaccagaagctgatcgccaatcagttcaactccgccatcggcaagatccaggactccctgtccagcaccgccagcgccctgggcaagctgcaggatgtggtgaatcagaacgcccaggccctgaataccctggtgaagcagctgtccagcaacttcggcgccatctccagcgtgctgaatgatatcctgagccgcctggacccaccggaggctgaggtgcagatcgataggctgatcaccggccgcctgcagtccctgcagacctacgtgacccagcagctgatcagggctgctgagatcagggccagcgccaatctggctgctaccaagatgtccgagtgcgtgctgggacagagcaagagggtggacttctgcggcaagggctaccacctgatgtccttcccacagagcgccccacacggagtggtgttcctgcacgtgacctacgtgccagcccaggagaagaacttcaccaccgctccagctatctgccacgatggcaaggctcacttcccacgcgagggcgtgttcgtgtccaacggcacccactggttcgtgacccagcgcaatttctacgagccccagatcatcaccaccgacaataccttcgtgagcggcaactgcgacgtggtcatcggaatcgtgaacaataccgtgtacgatcccctgcagccagagctggactccttcaaggaggagctggataagtacttcaagaatcacaccagccccgacgtggatctgggcgacatctccggcatcaatgccagcgtggtgaacatccagaaggagatcgaccgcctgaacgaggtggccaagaatctgaacgagtccctgatcgatctgcaggagctgggcaagtacgagcagtacatcaagggatccggcataaaaaggatgaagcaaatcgaggataaaattgaggaaatcgagtcgaagcaaaaaaaaatcgagaatgagatagcgcgaatcaagaagataaagggccaccaccaccaccaccac

SEQ ID NO: 24 - protein, 20 aa, Severe acute respiratory syndrome coronavirus 2
ELGKYEQYIKWPWYIWLGFI

SEQ ID NO: 25 - protein, 1240 aa, Artificial Sequence
  Description of Artificial Sequence Synthetic polypeptide
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPAGAGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKGSGIKRMKQIEDKIEEIESKQKKIENEIARIKKIKGHHHHHH

SEQ ID NO: 26 - protein, 1253 aa, Artificial Sequence
  Description of Artificial Sequence Synthetic polypeptide
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPAGAGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKGSGIKRMKQIEDKIEEIESKQKKIENEIARIKKIKGHHHHHH

SEQ ID NO: 27 - protein, 19 aa, Severe acute respiratory syndrome coronavirus 2
ELGKYEQYIKWPWYIWLGF

SEQ ID NO: 28 - protein, 18 aa, Severe acute respiratory syndrome coronavirus 2
ELGKYEQYIKWPWYIWLG

SEQ ID NO: 29 - protein, 17 aa, Severe acute respiratory syndrome coronavirus 2
ELGKYEQYIKWPWYIWL

SEQ ID NO: 30 - protein, 16 aa, Severe acute respiratory syndrome coronavirus 2
ELGKYEQYIKWPWYIW

SEQ ID NO: 31 - protein, 15 aa, Severe acute respiratory syndrome coronavirus 2
ELGKYEQYIKWPWYI

SEQ ID NO: 32 - protein, 14 aa, Severe acute respiratory syndrome coronavirus 2
ELGKYEQYIKWPWY

SEQ ID NO: 33 - protein, 13 aa, Severe acute respiratory syndrome coronavirus 2
ELGKYEQYIKWPW

SEQ ID NO: 34 - protein, 12 aa, Severe acute respiratory syndrome coronavirus 2
ELGKYEQYIKWP

SEQ ID NO: 35 - protein, 11 aa, Severe acute respiratory syndrome coronavirus 2
ELGKYEQYIKW

SEQ ID NO: 36 - protein, 10 aa, Severe acute respiratory syndrome coronavirus 2
ELGKYEQYIK

SEQ ID NO: 37 - protein, 9 aa, Severe acute respiratory syndrome coronavirus 2
ELGKYEQYI

SEQ ID NO: 38 - protein, 8 aa, Severe acute respiratory syndrome coronavirus 2
ELGKYEQY

SEQ ID NO: 39 - protein, 7 aa, Severe acute respiratory syndrome coronavirus 2
ELGKYEQ

SEQ ID NO: 40 - protein, 6 aa, Severe acute respiratory syndrome coronavirus 2
ELGKYE

SEQ ID NO: 41 - protein, 5 aa, Severe acute respiratory syndrome coronavirus 2
ELGKY

SEQ ID NO: 42 - protein, 4 aa, Severe acute respiratory syndrome coronavirus 2
ELGK

SEQ ID NO: 43 - protein, 8 aa, Artificial Sequence
  Description of Artificial Sequence Synthetic 8xHis tag
HHHHHHHH

SEQ ID NO: 44 - protein, 4 aa, Severe acute respiratory syndrome coronavirus 2
RRAR

(19) United States
(12) Patent Application Publication
(10) Pub. No.: US 2022/0324918 A1
(43) Pub. Date: Oct. 13, 2022

(54) SARS-CoV-2 SPIKE ECTODOMAIN POLYPEPTIDES AND COMPOSITIONS AND METHODS THEREOF

(71) Applicant: REGENTS OF THE UNIVERSITY OF MINNESOTA, Minneapolis, MN (US)

(72) Inventors: Frank Jonathan Lee, Minneapolis, MN (US); Yushun Wan, Chongqing City (CN)

(73) Assignee: REGENTS OF THE UNIVERSITY OF MINNESOTA, Minneapolis, MN (US)

(21) Appl. No.: 17/706,919

(22) Filed: Mar. 29, 2022

Related U.S. Application Data
(60) Provisional application No. 63/167,773, filed on Mar. 30, 2021.

(51) Int. Cl. C07K 14/165; G01N 33/569

(52) U.S. Cl. CPC C07K 14/165; G01N 33/56983; C07K 2319/01

(57) ABSTRACT

The present disclosure provides engineered SARS-CoV-2 spike ectodomain polypeptides and cells for producing such a polypeptide, as well as compositions and methods thereof.

### CROSS REFERENCE TO RELATED APPLICATION

This application claims priority to U.S. Provisional Application No. 63/167,773 filed on 30 Mar. 2021. The entire content of the application referenced above is hereby incorporated by reference herein.

### GOVERNMENT FUNDING

This invention was made with government support under A1089728 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on May 9, 2022, is named 09531_533US1_SL.txt and is 181,659 bytes in size.

### BACKGROUND OF THE INVENTION

Coronaviruses, which cause disease in mammals and birds, are a group of enveloped viruses that have a positive-sense single-stranded RNA genome and a nucleocapsid of helical symmetry. Their genome encodes four major structural proteins: spike (S), membrane (M), envelope (E) and nucleocapsid (N). In humans, coronaviruses cause respiratory tract infections that can range from mild to lethal. Mild illnesses include some cases of the common cold, while more lethal varieties can cause COVID-19, SARS and MERS. SARS-coronavirus 2 (SARS-CoV-2) causes COVID-19, a disease that spread rapidly and created a global pandemic. A current barrier to pandemic control is the lack of ability to produce stable and reliable viral proteins with high yield for testing of infection or acquired immunity. Thus, there is a need for new approaches for developing and engineering such tools, as well as COVID-19 specific diagnostic tests.

### SUMMARY OF THE INVENTION

Certain embodiments of the invention provide a method for detecting an anti-SARS-CoV-2 antibody in a sample, the method comprising 1) contacting the sample with a polypeptide comprising a SARS-CoV-2 spike ectodomain amino acid sequence comprising a D614G mutation, under conditions suitable for an anti-SARS-CoV-2 antibody to bind to the polypeptide; and 2) detecting the presence of an anti-SARS-CoV-2 antibody bound to the polypeptide. In certain embodiments, the polypeptide further comprises one or more mutations in the furin cleavage motif [R682-R683-A684-R685 (SEQ ID NO: 44)], wherein the mutation(s) renders the polypeptide resistant to furin cleavage.

Certain embodiments of the invention provide a method for detecting a molecule associated with an immune response to SARS-CoV-2 or to a SARS-CoV-2 vaccine in an animal (e.g., human), the method comprising 1) contacting a sample from the animal with a polypeptide comprising a SARS-CoV-2 spike ectodomain amino acid sequence comprising a D614G mutation, under conditions suitable for the molecule to bind to the polypeptide; and 2) detecting the presence of the molecule bound to the polypeptide.

Certain embodiments of the invention provide a method of diagnosing an animal as having or having had a SARS-CoV2 infection, the method comprising 1) obtaining a biological sample from the animal; 2) contacting the sample with a polypeptide comprising a SARS-CoV-2 spike ectodomain amino acid sequence comprising a D614G mutation and detecting whether anti-SARS-CoV-2 antibodies are present in the sample; and 3) diagnosing the animal as having or having had a SARS-CoV2 infection when anti-SARS-CoV-2 antibodies are detected.

Certain embodiments of the invention provide a polypeptide comprising a SARS-CoV-2 spike ectodomain amino acid sequence comprising R682A, R683G and R685G mutations.

Certain embodiments of the invention provide a composition comprising a polypeptide as described herein and a carrier.

Certain embodiments of the invention provide a diagnostic composition comprising a polypeptide or trimerized polypeptide as described herein and a substrate, wherein the polypeptide or trimerized polypeptide is immobilized on the substrate.

Certain embodiments of the invention provide a method for detecting an anti-SARS-CoV-2 antibody in a sample, the method comprising 1) contacting the sample with a polypeptide as described herein, under conditions suitable for an anti-SARS-CoV-2 antibody to bind to the polypeptide; and 2) detecting the presence of an anti-SARS-CoV-2 antibody bound to the polypeptide.

Certain embodiments of the invention provide a method for detecting a molecule associated with an immune response to SARS-CoV-2 or to a SARS-CoV-2 vaccine in an animal (e.g., human), the method comprising 1) contacting a sample from the animal with a polypeptide as described herein, under conditions suitable for the molecule to bind to the polypeptide; and 2) detecting the presence of the molecule bound to the polypeptide.

Certain embodiments of the invention provide a method of diagnosing an animal as having or having had a SARS-CoV2 infection, the method comprising 1) obtaining a biological sample from the animal; 2) contacting the sample with a polypeptide as described herein and detecting whether anti-SARS-CoV-2 antibodies are present in the sample; and 3) diagnosing the animal as having or having had a SARS-CoV2 infection when anti-SARS-CoV-2 antibodies are detected.

Certain embodiments of the invention provide a kit comprising an isolated or purified SARS-CoV-2 spike ectodomain polypeptide as described herein, packaging material, and instructions for using the polypeptide in a diagnostic method as described herein.

Certain embodiments of the invention provide an isolated polynucleotide comprising a nucleotide sequence encoding the polypeptide as described herein.

Certain embodiments of the invention provide an expression cassette comprising a promoter operably linked to the polynucleotide as described herein.

Certain embodiments of the invention provide a vector comprising the polynucleotide as described herein or the expression cassette as described herein.

Certain embodiments of the invention provide a cell comprising the polynucleotide as described herein, the expression cassette as described herein or the vector as described herein.

Certain embodiments of the invention provide a method of making a cell as described herein, the method comprising transfecting or transducing the cell with the polynucleotide as described herein, the expression cassette as described herein or the vector as described herein.

Certain embodiments of the invention provide a method of producing a polypeptide, the method comprising transfecting or transducing a cell with the polynucleotide as described herein, the expression cassette as described herein or the vector as described herein.

Certain embodiments of the invention provide a method of producing a polypeptide, the method comprising culturing a cell as described herein under conditions appropriate for polypeptide expression.

Certain embodiments of the invention provide a polypeptide produced by a method as described herein.

The invention also provides processes and intermediates disclosed herein that are useful for preparing polypeptides and cells of the invention, as well as compositions described herein.

### BRIEF DESCRIPTION OF THE FIGURES

FIG. 1. Schematic graph of SARS-CoV-2 Spike protein, full-length sequence, and two embodiments of engineered SARS-CoV-2 spike ectodomain polypeptide.

FIG. 2. One embodiment of a vector comprising an expression cassette that comprises a nucleic acid sequence encoding an engineered SARS-CoV-2 spike ectodomain polypeptide described herein. Figure discloses SEQ ID NO: 20.

FIGS. 3A-3B. Purification of an engineered SARS-CoV-2 spike ectodomain. FIG. 3A elution profile of gel filtration chromatography of engineered SARS-CoV-2 spike ectodomain.

FIG. 3B SDS-PAGE analysis of purified engineered SARS-CoV-2 spike ectodomain.

FIG. 4. Antibody titers in sera of RBD- or control spike ectodomain-administered mice as measured by ELISA using RBD or engineered spike ectodomain as an ELISA antigen.

FIG. 5. Comparison of RBD and spike ectodomain as antigens in COVID-19 diagnostic assays. ELISA was performed to detect the SARS-CoV-2-specific antibody titers in COVID-19 patients (samples #1-6), vaccinated people (samples #7-12, with #10 as a control), and pre-pandemic samples. Two antigens, RBD and spike ectodomain, were used for head-to-head comparisons (equal molar amounts of the antigens were coated).

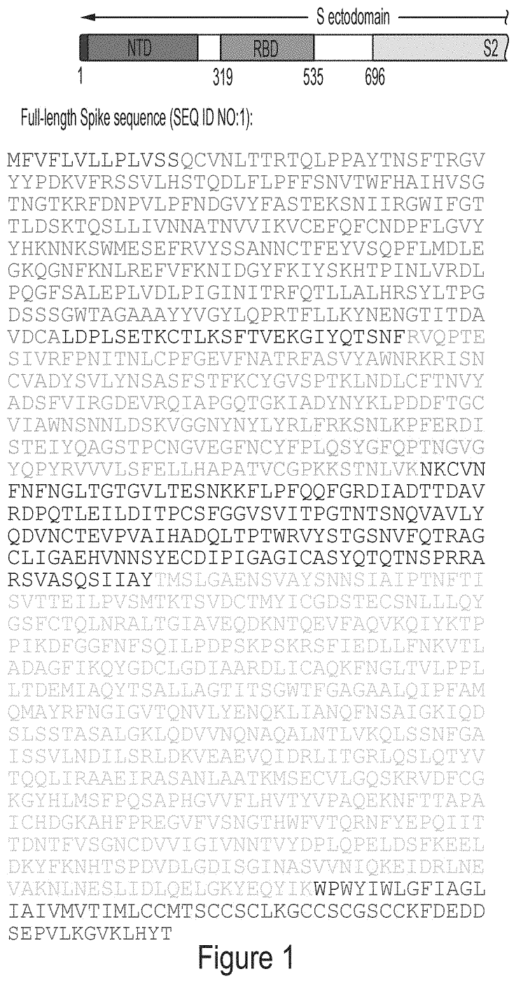

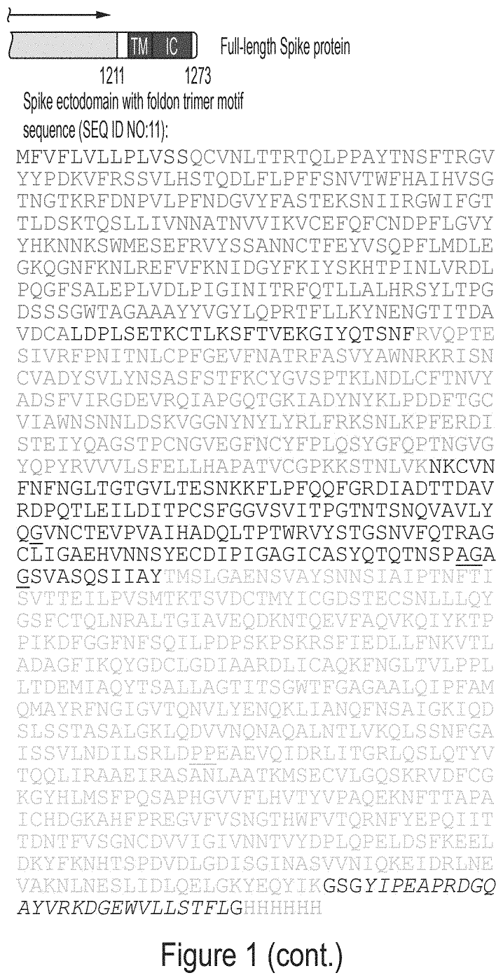

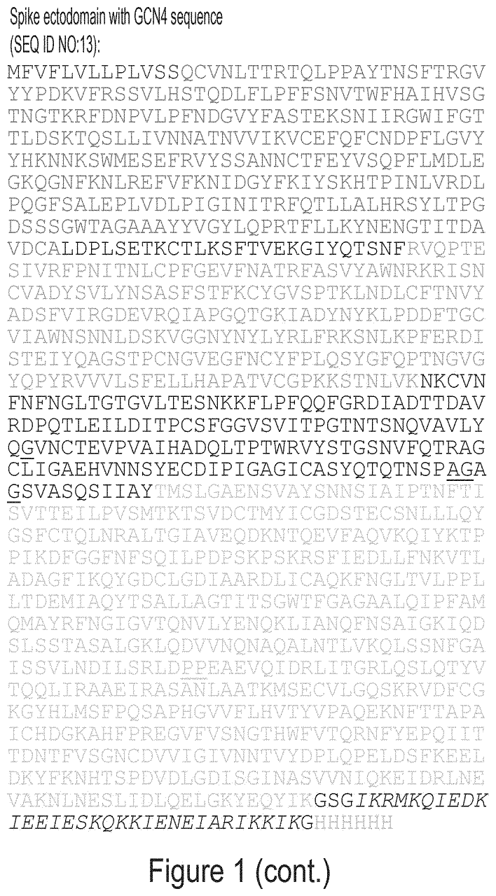

### DETAILED DESCRIPTION

Coronaviruses (CoV) are positive sense, single-stranded RNA viruses whose genome encodes 4 major structural proteins: Spike (S), membrane (M), envelope (E), and nucleocapsid (N). The S protein mediates binding of the virus to the host cell receptor, and fusion between the two membranes allows for viral entry into the host cell. The S protein of SARS-CoV-2 has two subunits, 51 and S2. The 51 subunit binds to the host receptor through its receptor-binding domain (RBD), allowing for conformational changes and membrane fusion. The Spike protein is a membrane anchored protein that has extracellular segment (ectodomain), transmembrane segment and intracellular segment.

The RBD and the N protein have traditionally been used as an antigen in COVID-19 diagnostics. However, the recent emergence of many SARS-CoV-2 variants raises concerns over the effectiveness of using the RBD in diagnosis of these SARS-CoV-2 variants. Additionally, compared to the RBD or the N protein, the spike ectodomain has the following advantages as a diagnostic antigen: (i) the N protein has relatively low specificity; (ii) the spike ectodomain contains more epitopes than the RBD, which makes the spike ectodomain a superior diagnostics antigen; (iii) the non-RBD regions in the spike ectodomain are relatively evolutionarily stable, allowing the spike protein to be an effective diagnostic antigen against different SARS-CoV-2 variants; and (iv) the spike ectodomain is uniquely important for detecting spike-targeting antibodies in people immunized by spike mRNA vaccines (i.e., those produced by Moderna and Pfizer). Despite these advantages, two limiting factors for the spike ectodomain are its instability and very low yield.

As described herein, these problems have been addressed through the development of an engineered SARS-CoV-2 spike ectodomain construct. In particular, described herein are certain engineered recombinant SARS-CoV-2 spike ectodomain polypeptides with improved yield and stability. In certain embodiments, the engineered spike ectodomain comprises a D614G mutation. As described herein, it was found that the D614G mutation surprisingly increases the yield of spike ectodomain production by about 20-fold (see, comparison made in Example 1). In addition, certain engineered polypeptides have enhanced stability and/or resistance against denaturation or proteolytic degradation. Accordingly, such engineered recombinant spike ectodomain polypeptides can be quickly obtained with a high yield and purity. Additionally, the resulting protein was shown to be properly folded and biologically active (e.g., it demonstrates high sensitivity in detecting spike-specific antibodies in diagnostic test such as ELISA) (see, e.g., the Examples describing an engineered spike ectodomain polypeptide comprising D614G and five other mutations). Production of an engineered spike ectodomain polypeptide as described herein may also be readily scaled up for commercial uses, thereby enabling the development of COVID-19 diagnostic assays.

Accordingly, provided herein are engineered spike ectodomain polypeptides for use in certain diagnostic methods, as well as certain diagnostic compositions.

### Engineered Polypeptides

Certain embodiments of the invention provide a polypeptide comprising a SARS-CoV-2 spike ectodomain amino acid sequence, or a fragment thereof, which comprises a D614G mutation and/or mutations that confer resistance to furin cleavage (e.g., R682A, R683G and R685G) for use in a diagnostic composition/method described herein. In certain embodiments, the polypeptide described herein is a recombinant polypeptide. In certain embodiments, the polypeptide described herein is isolated or purified.

As used herein, the term “spike ectodomain” refers to the extracellular segment of the Spike protein, which lacks the transmembrane segment and intracellular segment (see, e.g., certain embodiments in Table 1). An embodiment of a wildtype spike ectodomain sequence is shown as SEQ ID NO:2 in Table 1.

In certain embodiments, the polypeptide comprises a D614G mutation. Without wishing to be bound by theory, it is believed that the D614G mutation may further expose the RBD. Additionally, this mutation was shown to increase the yield of spike ectodomain production by about 20-fold (see, comparison made in Example 1).

In certain embodiments, an engineered polypeptide as described herein possesses improved stability and/or resistance to certain proteinase digestion. In certain embodiments, the polypeptide is resistant to furin cleavage. For example, the furin cleavage motif [R682-R683-A684-R685 (SEQ ID NO: 44)] of the spike ectodomain may be mutated to confer resistance to furin mediated cleavage. In certain embodiments, the polypeptide comprises one, two, three, or four mutations in the furin cleavage motif [R682-R683-A684-R685 (SEQ ID NO: 44)]. In certain embodiments, the polypeptide comprises three mutations in the furin cleavage motif [R682-R683-A684-R685 (SEQ ID NO: 44)]. In certain embodiments, the polypeptide comprises R682A, R683G and R685G mutations.

In certain embodiments, the polypeptide further comprises one or more mutations that stabilize the spike ectodomain polypeptide. For example, in certain embodiments, the polypeptide comprises K986P and V987P mutations.

Thus, in certain embodiments, a polypeptide as described herein comprises a D614G mutation. In certain embodiments, a polypeptide as described herein comprises D614G, R682A, R683G, and R685G mutations. In certain embodiments, a polypeptide as described herein comprises D614G, K986P, and V987P mutations. In certain embodiments, a polypeptide as described herein comprises R682A, R683G, R685G mutations. In certain embodiments, a polypeptide as described herein comprises R682A, R683G, R685G, K986P, and V987P mutations. In certain embodiments, a polypeptide as described herein comprises D614G, R682A, R683G, R685G, K986P, and V987P mutations.

In certain embodiments, the spike ectodomain amino acid sequence is between about 1,180 to about 1,230 amino acids in length. In certain embodiments, the spike ectodomain amino acid sequence is between about 1,185 to about 1,225 amino acids in length. In certain embodiments, the spike ectodomain amino acid sequence is between about 1,190 to about 1,220 amino acids in length. In certain embodiments, the spike ectodomain amino acid sequence is between about 1,192 to about 1,218 amino acids in length. In certain embodiments, the spike ectodomain amino acid sequence is between about 1,194 to about 1,216 amino acids in length. In certain embodiments, the spike ectodomain amino acid sequence is between about 1,194 to about 1,214 amino acids in length. In certain embodiments, the spike ectodomain amino acid sequence is between about 1,188 to about 1,208 amino acids in length. In certain embodiments, the spike ectodomain amino acid sequence is between about 1,190 to about 1,206 amino acids in length. In certain embodiments, the spike ectodomain amino acid sequence is between about 1,192 to about 1,204 amino acids in length. In certain embodiments, the spike ectodomain amino acid sequence is between about 1,194 to about 1,202 amino acids in length. In certain embodiments, the spike ectodomain amino acid sequence is between about 1,196 to about 1,200 amino acids in length. In certain embodiments, the spike ectodomain amino acid sequence is about 1,198 amino acids in length. In certain embodiments, the spike ectodomain amino acid sequence is between about 1,201 to about 1,221 amino acids in length. In certain embodiments, the spike ectodomain amino acid sequence is between about 1,203 to about 1,219 amino acids in length. In certain embodiments, the spike ectodomain amino acid sequence is between about 1,205 to about 1,217 amino acids in length. In certain embodiments, the spike ectodomain amino acid sequence is between about 1,207 to about 1,215 amino acids in length. In certain embodiments, the spike ectodomain amino acid sequence is between about 1,209 to about 1,213 amino acids in length. In certain embodiments, the spike ectodomain amino acid sequence is about 1,211 amino acids in length.

In certain embodiments, the SARS-CoV-2 spike ectodomain amino acid sequence comprises an amino acid sequence having at least about 80% sequence identity to SEQ ID NO:3 and comprises 1) a D614G mutation; and/or 2) R682A, R683G and R685G mutations, and optionally, 3) K986P, and V987P mutations. In certain embodiments, the SARS-CoV-2 spike ectodomain comprises an amino acid sequence having at least about 81%, 82%, 83%, 84%, 85%, 86%, 87%, 88%, 89%, 90%, 91%, 92%, 93%, 94%, 95%, 96%, 97%, 98%, 99% or 100% identity to SEQ ID NO:3; and comprises 1) a D614G mutation; and/or 2) R682A, R683G and R685G mutations, and optionally, 3) K986P, and V987P mutations. In certain embodiments, the SARS-CoV-2 spike ectodomain comprises an amino acid sequence having at least about 85% sequence identity to SEQ ID NO: 3; and comprises 1) a D614G mutation; and/or 2) R682A, R683G and R685G mutations, and optionally, 3) K986P, and V987P mutations. In certain embodiments, the SARS-CoV-2 spike ectodomain comprises an amino acid sequence having at least about 90% sequence identity to SEQ ID NO: 3; and comprises 1) a D614G mutation; and/or 2) R682A, R683G and R685G mutations, and optionally, 3) K986P, and V987P mutations. In certain embodiments, the SARS-CoV-2 spike ectodomain comprises an amino acid sequence having at least about 95% sequence identity to SEQ ID NO: 3; and comprises 1) a D614G mutation; and/or 2) R682A, R683G and R685G mutations, and optionally, 3) K986P, and V987P mutations.

In certain embodiments, the SARS-CoV-2 spike ectodomain amino acid sequence comprises an amino acid sequence having at least about 80% sequence identity to SEQ ID NO:3, and comprises 1) a D614G mutation; and/or 2) R682A, R683G and R685G mutations, and optionally, 3) K986P, and V987P mutations. In certain embodiments, the SARS-CoV-2 spike ectodomain amino acid sequence is further linked (e.g., through a peptide bond) to 1 to 20 (e.g., consecutive) amino acids provided in a sequence corresponding to SEQ ID NO:24. For example, in certain embodiments, the SARS-CoV-2 spike ectodomain (e.g., SEQ ID NO:3) carboxy-terminus is further linked to: ELGKYEQYIKWPWYIWLGFI (SEQ ID NO: 24), ELGKYEQYIKWPWYIWLGF (SEQ ID NO: 27), ELGKYEQYIKWPWYIWLG (SEQ ID NO: 28), ELGKYEQYIKWPWYIWL (SEQ ID NO: 29), ELGKYEQYIKWPWYIW (SEQ ID NO: 30), ELGKYEQYIKWPWYI (SEQ ID NO: 31), ELGKYEQYIKWPWY (SEQ ID NO: 32), ELGKYEQYIKWPW (SEQ ID NO: 33), ELGKYEQYIKWP (SEQ ID NO: 34), ELGKYEQYIKW (SEQ ID NO: 35), ELGKYEQYIK (SEQ ID NO: 36), ELGKYEQYI (SEQ ID NO: 37), ELGKYEQY (SEQ ID NO: 38), ELGKYEQ (SEQ ID NO: 39), ELGKYE (SEQ ID NO: 40), ELGKY (SEQ ID NO: 41), ELGK (SEQ ID NO: 42), ELG, EL, or E.

In certain embodiments, the SARS-CoV-2 spike ectodomain amino acid sequence comprises an amino acid sequence having at least about 80% sequence identity to any one of SEQ ID NOs:4-7. In certain embodiments, the SARS-CoV-2 spike ectodomain comprises an amino acid sequence having at least about 81%, 82%, 83%, 84%, 85%, 86%, 87%, 88%, 89%, 90%, 91%, 92%, 93%, 94%, 95%, 96%, 97%, 98%, 99% or 100% identity to any one of SEQ ID NOs:4-7. In certain embodiments, the SARS-CoV-2 spike ectodomain comprises an amino acid sequence having at least about 85% sequence identity to any one of SEQ ID NOs: 4-7. In certain embodiments, the SARS-CoV-2 spike ectodomain comprises an amino acid sequence having at least about 90% sequence identity to SEQ ID NOs: 4-7. In certain embodiments, the SARS-CoV-2 spike ectodomain comprises an amino acid sequence having at least about 95% sequence identity to any one of SEQ ID NOs: 4-7. In certain embodiments, the SARS-CoV-2 spike ectodomain comprises an amino acid sequence having at least about 99% sequence identity to any one of SEQ ID NOs: 4-7. In certain embodiments, the SARS-CoV-2 spike ectodomain comprises SEQ ID NOs: 4-7. In certain embodiments, the SARS-CoV-2 spike ectodomain consists of SEQ ID NOs: 4-7.

In certain embodiments, the SARS-CoV-2 spike ectodomain comprises an amino acid sequence having at least about 80% sequence identity to SEQ ID NO:4. In certain embodiments, the SARS-CoV-2 spike ectodomain comprises an amino acid sequence having at least about 81%, 82%, 83%, 84%, 85%, 86%, 87%, 88%, 89%, 90%, 91%, 92%, 93%, 94%, 95%, 96%, 97%, 98%, 99% or 100% identity to SEQ ID NO:4. In certain embodiments, the SARS-CoV-2 spike ectodomain comprises an amino acid sequence having at least about 85% sequence identity to SEQ ID NO:4. In certain embodiments, the SARS-CoV-2 spike ectodomain comprises an amino acid sequence having at least about 90% sequence identity to SEQ ID NO:4. In certain embodiments, the SARS-CoV-2 spike ectodomain comprises an amino acid sequence having at least about 95% sequence identity to SEQ ID NO:4. In certain embodiments, the SARS-CoV-2 spike ectodomain comprises an amino acid sequence having at least about 99% sequence identity to SEQ ID NO:4. In certain embodiments, the SARS-CoV-2 spike ectodomain comprises SEQ ID NO:4. In certain embodiments, the SARS-CoV-2 spike ectodomain consists of SEQ ID NO:4.

In certain embodiments, the SARS-CoV-2 spike ectodomain comprises an amino acid sequence having at least about 80% sequence identity to SEQ ID NO:7. In certain embodiments, the SARS-CoV-2 spike ectodomain comprises an amino acid sequence having at least about 81%, 82%, 83%, 84%, 85%, 86%, 87%, 88%, 89%, 90%, 91%, 92%, 93%, 94%, 95%, 96%, 97%, 98%, 99% or 100% identity to SEQ ID NO:7. In certain embodiments, the SARS-CoV-2 spike ectodomain comprises an amino acid sequence having at least about 85% sequence identity to SEQ ID NO:7. In certain embodiments, the SARS-CoV-2 spike ectodomain comprises an amino acid sequence having at least about 90% sequence identity to SEQ ID NO:7. In certain embodiments, the SARS-CoV-2 spike ectodomain comprises an amino acid sequence having at least about 95% sequence identity to SEQ ID NO:7. In certain embodiments, the SARS-CoV-2 spike ectodomain comprises an amino acid sequence having at least about 99% sequence identity to SEQ ID NO:7. In certain embodiments, the SARS-CoV-2 spike ectodomain comprises SEQ ID NO:7. In certain embodiments, the SARS-CoV-2 spike ectodomain consists of SEQ ID NO:7.

In certain embodiments, a polypeptide as described herein comprises a trimerization motif, such as a foldon trimer motif or a GCN4 motif. Accordingly, a polypeptide described herein comprising a SARS-CoV-2 spike ectodomain may assemble into a trimer, thereby providing a timeric protein. In certain embodiments, the polypeptide described herein is operably linked to a trimerization motif. For example, a trimerization motif may be fused either directly or indirectly via a linker group to the N-terminus or C-terminus of a spike ectodomain amino acid sequence described herein (e.g., fused to the C-terminus). In certain embodiments, the trimerization motif is a foldon trimer motif (e.g., from bacteriophage T4 phagehead fibritin [Escherichia virus T4]). In certain embodiments, the foldon trimer motif comprises/consists of SEQ ID NO: 16. In certain embodiments, the trimerization motif is a GCN4 motif (e.g., from yeast [Saccharomyces cerevisiae S288C]). In certain embodiments, the GCN4 motif comprises/consists of SEQ ID NO: 17.

In certain embodiments, a spike ectodomain amino acid sequence described herein may be directly linked to a trimerization motif. In certain embodiments, a spike ectodomain amino acid sequence described herein may be operably linked to a trimerization motif via a linker group, such as a peptide linker. While the linker group may vary, it should not interfere with the function of the spike ectodomain or the trimerization motif. For example, the linker can be a flexible peptide linker such as a glycine rich linker. In certain embodiments, the linker is a GS rich amino acid sequence. In certain embodiments, the linker is a di-peptide, such as GS. In certain embodiments, the linker is a GSG (SEQ ID NO: 15).

In certain embodiments, the linker group is about 1 to about 30 amino acids in length. In certain embodiments, the linker group is about 1 to about 25 amino acids in length. In certain embodiments, the linker group is about 1 to about 20 amino acids in length. In certain embodiments, the linker group is about 1 to about 15 amino acids in length. In certain embodiments, the linker group is about 1 to about 12 amino acids in length. In certain embodiments, the linker group is about 1 to about 10 amino acids in length. In certain embodiments, the linker group is about 1 to about 9 amino acids in length. In certain embodiments, the linker group is about 1 to about 8 amino acids in length. In certain embodiments, the linker group is about 1 to about 7 amino acids in length. In certain embodiments, the linker group is about 1 to about 6 amino acids in length. In certain embodiments, the linker group is about 1 to about 5 amino acids in length. In certain embodiments, the linker group is about 1 to about 4 amino acids in length. In certain embodiments, the linker group is about 1 to about 3 amino acids in length. In certain embodiments, the linker group is about 2 amino acids in length. In certain embodiments, the linker group is about 1 amino acid in length.

In certain embodiments, a polypeptide as described herein may be further operably linked (e.g., either directly or through a linker group) to one or more detectable markers, such as a protein or peptide tag or a fluorescent tag. In certain embodiments, a polypeptide as described herein may be further operably linked (e.g., directly or through a linker group) to one or more protein or peptide tags that are useful for purification (e.g., an epitope tag or an affinity tag).

In certain embodiments, a polypeptide as described herein is operably linked to a protein or peptide tag. In certain embodiments, the peptide tag is an affinity tag. In certain embodiments, the affinity tag is a poly(His) tag, FLAG, 3×FLAG, c-Myc, Fc tag or a hemagglutinin tag (e.g. HA). In certain embodiments, the affinity tag is a poly(His) tag. In certain embodiments, the affinity tag is a 6×His tag (SEQ ID NO: 20). In certain embodiments, the affinity tag is an 8×His tag (SEQ ID NO: 43).

In some embodiments, the protein or peptide tag is operably linked to the N-terminal end and/or the C-terminal end of the polypeptide described herein (e.g., to the N- or C-terminus of the spike ectodomain amino acid sequence, such as the C-terminus).

In certain embodiments, the polypeptide (e.g., the spike ectodomain amino acid sequence) is directly linked to a protein or peptide tag. In certain embodiments, the polypeptide (e.g., the spike ectodomain amino acid sequence) is linked to a protein or peptide tag via a linker group. While the linker group may vary, it should not interfere with the function of the polypeptide (e.g., the spike ectodomain). For example, the linker can be a flexible peptide linker such as a glycine rich linker. In certain embodiments, the linker is a GS rich amino acid sequence. In certain embodiments, the linker is a di-peptide, such as GS. In certain embodiments, the linker is a GSG (SEQ ID NO: 15). In certain embodiments, the linker group is a single Glycine. In certain embodiments, the linker group comprises a trimerization motif described herein. For example, in certain embodiments, the linker comprises/consists of SEQ ID NO: 18 or 19.

In certain embodiments, the linker group is about 1 to about 50 amino acids in length. In certain embodiments, the linker group is about 1 to about 45 amino acids in length. In certain embodiments, the linker group is about 1 to about 40 amino acids in length. In certain embodiments, the linker group is about 1 to about 35 amino acids in length. In certain embodiments, the linker group is about 1 to about 30 amino acids in length. In certain embodiments, the linker group is about 1 to about 25 amino acids in length. In certain embodiments, the linker group is about 1 to about 20 amino acids in length. In certain embodiments, the linker group is about 1 to about 15 amino acids in length. In certain embodiments, the linker group is about 1 to about 12 amino acids in length. In certain embodiments, the linker group is about 1 to about 10 amino acids in length. In certain embodiments, the linker group is about 1 to about 9 amino acids in length. In certain embodiments, the linker group is about 1 to about 8 amino acids in length. In certain embodiments, the linker group is about 1 to about 7 amino acids in length. In certain embodiments, the linker group is about 1 to about 6 amino acids in length. In certain embodiments, the linker group is about 1 to about 5 amino acids in length. In certain embodiments, the linker group is about 1 to about 4 amino acids in length. In certain embodiments, the linker group is about 1 to about 3 amino acids in length. In certain embodiments, the linker group is about 2 amino acids in length. In certain embodiments, the linker group is about 1 amino acid in length. In certain embodiments, the linker group is about 30 amino acids in length. In certain embodiments, the linker group is about 32 amino acids in length.

In certain embodiments, the polypeptide comprises an amino acid sequence having at least about 80% sequence identity to SEQ ID NO:8, 10, 12, or 25. In certain embodiments, the polypeptide comprises an amino acid sequence having at least about 81%, 82%, 83%, 84%, 85%, 86%, 87%, 88%, 89%, 90%, 91%, 92%, 93%, 94%, 95%, 96%, 97%, 98%, 99% or 100% identity to SEQ ID NO: 8, 10, 12, or 25. In certain embodiments, the polypeptide comprises an amino acid sequence having at least about 85% sequence identity to SEQ ID NO: 8, 10, 12, or 25. In certain embodiments, the polypeptide comprises an amino acid sequence having at least about 90% sequence identity to SEQ ID NO: 8, 10, 12, or 25. In certain embodiments, the polypeptide comprises an amino acid sequence having at least about 95% sequence identity to SEQ ID NO: 8, 10, 12, or 25. In certain embodiments, the polypeptide comprises an amino acid sequence having at least about 99% sequence identity to SEQ ID NO: 8, 10, 12, or 25. In certain embodiments, the polypeptide comprises SEQ ID NO: 8, 10, 12, or 25. In certain embodiments, the polypeptide consists of SEQ ID NO: 8, 10, 12, or 25.

In certain embodiments, the polypeptide comprises an amino acid sequence having at least about 80% sequence identity to SEQ ID NO:8. In certain embodiments, the polypeptide comprises an amino acid sequence having at least about 81%, 82%, 83%, 84%, 85%, 86%, 87%, 88%, 89%, 90%, 91%, 92%, 93%, 94%, 95%, 96%, 97%, 98%, 99% or 100% identity to SEQ ID NO:8. In certain embodiments, the polypeptide comprises an amino acid sequence having at least about 85% sequence identity to SEQ ID NO:8. In certain embodiments, the polypeptide comprises an amino acid sequence having at least about 90% sequence identity to SEQ ID NO:8. In certain embodiments, the polypeptide comprises an amino acid sequence having at least about 95% sequence identity to SEQ ID NO:8. In certain embodiments, the polypeptide comprises an amino acid sequence having at least about 99% sequence identity to SEQ ID NO:8. In certain embodiments, the polypeptide comprises SEQ ID NO:8. In certain embodiments, the polypeptide consists of SEQ ID NO:8.

In certain embodiments, the polypeptide comprises an amino acid sequence having at least about 80% sequence identity to SEQ ID NO:10. In certain embodiments, the polypeptide comprises an amino acid sequence having at least about 81%, 82%, 83%, 84%, 85%, 86%, 87%, 88%, 89%, 90%, 91%, 92%, 93%, 94%, 95%, 96%, 97%, 98%, 99% or 100% identity to SEQ ID NO:10. In certain embodiments, the polypeptide comprises an amino acid sequence having at least about 85% sequence identity to SEQ ID NO:10. In certain embodiments, the polypeptide comprises an amino acid sequence having at least about 90% sequence identity to SEQ ID NO:10. In certain embodiments, the polypeptide comprises an amino acid sequence having at least about 95% sequence identity to SEQ ID NO:10. In certain embodiments, the polypeptide comprises an amino acid sequence having at least about 99% sequence identity to SEQ ID NO:10. In certain embodiments, the polypeptide comprises SEQ ID NO:10. In certain embodiments, the polypeptide consists of SEQ ID NO:10.

In certain embodiments, the polypeptide comprises an amino acid sequence having at least about 80% sequence identity to SEQ ID NO:12. In certain embodiments, the polypeptide comprises an amino acid sequence having at least about 81%, 82%, 83%, 84%, 85%, 86%, 87%, 88%, 89%, 90%, 91%, 92%, 93%, 94%, 95%, 96%, 97%, 98%, 99% or 100% identity to SEQ ID NO:12. In certain embodiments, the polypeptide comprises an amino acid sequence having at least about 85% sequence identity to SEQ ID NO:12. In certain embodiments, the polypeptide comprises an amino acid sequence having at least about 90% sequence identity to SEQ ID NO:12. In certain embodiments, the polypeptide comprises an amino acid sequence having at least about 95% sequence identity to SEQ ID NO:12. In certain embodiments, the polypeptide comprises an amino acid sequence having at least about 99% sequence identity to SEQ ID NO:12. In certain embodiments, the polypeptide comprises SEQ ID NO:12. In certain embodiments, the polypeptide consists of SEQ ID NO:12.

In certain embodiments, the polypeptide comprises an amino acid sequence having at least about 80% sequence identity to SEQ ID NO:25. In certain embodiments, the polypeptide comprises an amino acid sequence having at least about 81%, 82%, 83%, 84%, 85%, 86%, 87%, 88%, 89%, 90%, 91%, 92%, 93%, 94%, 95%, 96%, 97%, 98%, 99% or 100% identity to SEQ ID NO:25. In certain embodiments, the polypeptide comprises an amino acid sequence having at least about 85% sequence identity to SEQ ID NO:25. In certain embodiments, the polypeptide comprises an amino acid sequence having at least about 90% sequence identity to SEQ ID NO:25. In certain embodiments, the polypeptide comprises an amino acid sequence having at least about 95% sequence identity to SEQ ID NO:25. In certain embodiments, the polypeptide comprises an amino acid sequence having at least about 99% sequence identity to SEQ ID NO:25. In certain embodiments, the polypeptide comprises SEQ ID NO:25. In certain embodiments, the polypeptide consists of SEQ ID NO:25.

In certain embodiments, the polypeptide does not comprise a spike transmembrane segment. In certain embodiments, the polypeptide does not comprise a spike intracellular segment. In certain embodiments, the polypeptide does not comprise a spike transmembrane segment or a spike intracellular segment. In certain embodiments, the polypeptide is a SAR-COV-2 spike ectodomain polypeptide as described herein that is optionally operably linked to a peptide tag (e.g., directly or through a linker group described herein, such as a linker group comprising a trimerization motif). In certain embodiments, the polypeptide consists of a spike ectodomain polypeptide described herein. In certain embodiments, the polypeptide consists of a spike ectodomain polypeptide as described herein and a trimerization motif. In certain embodiments, the polypeptide consists of a spike ectodomain polypeptide as described herein, a linker group, and a trimerization motif. In certain embodiments, the polypeptide consists of a spike ectodomain polypeptide as described herein, an optional linker group (e.g., comprising a trimerization motif), and a peptide tag.

In certain embodiments, the polypeptide further comprises a signal peptide. In certain embodiments, the signal peptide is a wildtype SARS-CoV-2 spike protein signal peptide (e.g., SEQ ID NO: 14). In certain embodiments, the signal peptide is cleaved from the polypeptide prior to secretion.

In certain embodiments, the signal peptide is operably linked to the N-terminus of the spike ectodomain amino acid sequence. In certain embodiments, the signal peptide is operably linked to the C-terminus of the spike ectodomain amino acid sequence.

In certain embodiments, the signal peptide is directly linked to the spike ectodomain amino acid sequence. In certain embodiments, the signal peptide is linked to the spike ectodomain amino acid sequence through a linker group. In certain embodiments, the linker group is a peptide linker.

In certain embodiments, the polypeptide comprises an amino acid sequence having at least about 80% identity to SEQ ID NO:9. In certain embodiments, the polypeptide comprises an amino acid sequence having at least about 81%, 82%, 83%, 84%, 85%, 86%, 87%, 88%, 89%, 90%, 91%, 92%, 93%, 94%, 95%, 96%, 97%, 98%, 99% or 100% identity to SEQ ID NO:9. In certain embodiments, the polypeptide comprises an amino acid sequence having at least about 85% sequence identity to SEQ ID NO:9. In certain embodiments, the polypeptide comprises an amino acid sequence having at least about 90% sequence identity to SEQ ID NO:9. In certain embodiments, the polypeptide comprises an amino acid sequence having at least about 95% sequence identity to SEQ ID NO:9. In certain embodiments, the polypeptide comprises an amino acid sequence having at least about 99% sequence identity to SEQ ID NO:9. In certain embodiments, the polypeptide comprises SEQ ID NO:9. In certain embodiments, the polypeptide consists of SEQ ID NO:9.

In certain embodiments, the polypeptide comprises an amino acid sequence having at least about 80% identity to SEQ ID NO:11. In certain embodiments, the polypeptide comprises an amino acid sequence having at least about 81%, 82%, 83%, 84%, 85%, 86%, 87%, 88%, 89%, 90%, 91%, 92%, 93%, 94%, 95%, 96%, 97%, 98%, 99% or 100% identity to SEQ ID NO:11. In certain embodiments, the polypeptide comprises an amino acid sequence having at least about 85% sequence identity to SEQ ID NO:11. In certain embodiments, the polypeptide comprises an amino acid sequence having at least about 90% sequence identity to SEQ ID NO:11. In certain embodiments, the polypeptide comprises an amino acid sequence having at least about 95% sequence identity to SEQ ID NO:11. In certain embodiments, the polypeptide comprises an amino acid sequence having at least about 99% sequence identity to SEQ ID NO:11. In certain embodiments, the polypeptide comprises SEQ ID NO:11. In certain embodiments, the polypeptide consists of SEQ ID NO:11.

In certain embodiments, the polypeptide comprises an amino acid sequence having at least about 80% identity to SEQ ID NO:13. In certain embodiments, the polypeptide comprises an amino acid sequence having at least about 81%, 82%, 83%, 84%, 85%, 86%, 87%, 88%, 89%, 90%, 91%, 92%, 93%, 94%, 95%, 96%, 97%, 98%, 99% or 100% identity to SEQ ID NO:13. In certain embodiments, the polypeptide comprises an amino acid sequence having at least about 85% sequence identity to SEQ ID NO:13. In certain embodiments, the polypeptide comprises an amino acid sequence having at least about 90% sequence identity to SEQ ID NO:13. In certain embodiments, the polypeptide comprises an amino acid sequence having at least about 95% sequence identity to SEQ ID NO:13. In certain embodiments, the polypeptide comprises an amino acid sequence having at least about 99% sequence identity to SEQ ID NO:13. In certain embodiments, the polypeptide comprises SEQ ID NO:13. In certain embodiments, the polypeptide consists of SEQ ID NO:13.

In certain embodiments, the polypeptide comprises an amino acid sequence having at least about 80% identity to SEQ ID NO:26. In certain embodiments, the polypeptide comprises an amino acid sequence having at least about 81%, 82%, 83%, 84%, 85%, 86%, 87%, 88%, 89%, 90%, 91%, 92%, 93%, 94%, 95%, 96%, 97%, 98%, 99% or 100% identity to SEQ ID NO:26. In certain embodiments, the polypeptide comprises an amino acid sequence having at least about 85% sequence identity to SEQ ID NO:26. In certain embodiments, the polypeptide comprises an amino acid sequence having at least about 90% sequence identity to SEQ ID NO:26. In certain embodiments, the polypeptide comprises an amino acid sequence having at least about 95% sequence identity to SEQ ID NO:26. In certain embodiments, the polypeptide comprises an amino acid sequence having at least about 99% sequence identity to SEQ ID NO:26. In certain embodiments, the polypeptide comprises SEQ ID NO:26. In certain embodiments, the polypeptide consists of SEQ ID NO:26.

In certain embodiments, the polypeptide is between about 1,180 to about 3,000 amino acids in length. In certain embodiments, the polypeptide is between about 1,180 to about 2,500 amino acids in length. In certain embodiments, the polypeptide is between about 1,180 to about 2,225 amino acids in length. In certain embodiments, the polypeptide is between about 1,180 to about 2,000 amino acids in length. In certain embodiments, the polypeptide is between about 1,180 to about 1,750 amino acids in length. In certain embodiments, the polypeptide is between about 1,180 to about 1,500 amino acids in length. In certain embodiments, the polypeptide is between about 1,180 to about 1,400 amino acids in length. In certain embodiments, the polypeptide is between about 1,180 to about 1,300 amino acids in length. In certain embodiments, the polypeptide is between about 1,180 to about 1,275 amino acids in length. In certain embodiments, the polypeptide is between about 1,180 to about 1,263 amino acids in length. In certain embodiments, the polypeptide is between about 1,190 to about 1,263 amino acids in length. In certain embodiments, the polypeptide is between about 1,200 to about 1,263 amino acids in length. In certain embodiments, the polypeptide is between about 1,210 to about 1,263 amino acids in length. In certain embodiments, the polypeptide is between about 1,220 to about 1,263 amino acids in length. In certain embodiments, the polypeptide is between about 1,225 to about 1,263 amino acids in length. In certain embodiments, the polypeptide is between about 1,230 to about 1,257 amino acids in length. In certain embodiments, the polypeptide is between about 1,248 to about 1,258 amino acids in length. In certain embodiments, the polypeptide is between about 1,251 to about 1,255 amino acids in length. In certain embodiments, the polypeptide is about 1,253 amino acids in length. In certain embodiments, the polypeptide is between about 1,242 to about 1,252 amino acids in length. In certain embodiments, the polypeptide is between about 1,245 to about 1,249 amino acids in length. In certain embodiments, the polypeptide is about 1,247 amino acids in length. In certain embodiments, the polypeptide is between about 1,235 to about 1,245 amino acids in length. In certain embodiments, the polypeptide is between about 1,238 to about 1,242 amino acids in length. In certain embodiments, the polypeptide is about 1,240 amino acids in length. In certain embodiments, the polypeptide is between about 1,229 to about 1,239 amino acids in length. In certain embodiments, the polypeptide is between about 1,232 to about 1,236 amino acids in length. In certain embodiments, the polypeptide is about 1,234 amino acids in length.

In certain embodiments, the polypeptide is capable of binding to angiotensin-converting enzyme 2 (ACE2) (e.g., with high affinity). In certain embodiments, ACE2 is human ACE2.

In some embodiments, a polypeptide described herein is prepared using recombinant methods. Accordingly, in certain embodiments, a polynucleotide (e.g., isolated polynucleotides) comprising a nucleic acid sequence encoding a polypeptide described herein is used to generate the polypeptide. In certain embodiments, the nucleic acid is operably linked to a promoter. In certain embodiments, the nucleic acid is comprised within an expression cassette, wherein the nucleic acid is operably linked to a promoter. Nucleic acids/expression cassettes encoding a polypeptide described herein can also be engineered into a vector (e.g., a vector described herein) for use in producing a polypeptide described herein.

In certain embodiments, a genetically modified cell may be used to produce a polypeptide described herein. Thus, in certain embodiments, a nucleic acid, expression cassette or vector (e.g., a vector described herein) encoding a polypeptide described herein may be used to transfect or transduce a cell (e.g., a cell described herein) to produce the polypeptide. In certain embodiments, the cell is a mammalian cell. In certain embodiments, the cell is a 293F cell. In certain embodiments, the cell is a cell described herein. In certain embodiments, the cell is stably transfected. In certain embodiments, the cell is stably transduced.

### A Polypeptide Immobilized on a Substrate

In certain embodiments, a polypeptide described herein is immobilized (e.g., covalently or non-covalently) on a substrate. Accordingly, certain embodiments provide a composition comprising a polypeptide described herein and a substrate, wherein the polypeptide is immobilized on the substrate.

In certain embodiments, the substrate is a solid substrate. In certain embodiments, a polypeptide described herein is coated on a substrate via passive adsorption. In certain embodiments, a polypeptide described herein is immobilized on a substrate via chemical conjugation or complexation. In certain embodiments, a polypeptide described herein is immobilized on a substrate via a capture antibody (e.g., an anti-His tag antibody).

In certain embodiments, the substrate is an ELISA plate (e.g., a polystyrene plate).

In certain embodiments, the substrate is a particle (e.g., a nanoparticle or microparticle). In certain embodiments, the particle is a metal particle (e.g., gold or silver). In certain embodiments, the particle is a magnetic particle (e.g., a paramagnetic nanoparticle or microparticle). In certain embodiments, the particle is a polymeric particle (e.g., polystyrene particle). In certain embodiments, the particle is a dextran coated particle.

In certain embodiments, the substrate is a chip (e.g., a surface plasmon resonance (SPR) sensor chip).

In certain embodiments, the substrate is a matrix. In certain embodiments, the substrate is a matrix (e.g., sugar- or acrylamide-based polymer resin and/or gel) in an affinity column for isolating or purifying a SARS-Cov-2 Spike binder agent (e.g., antibody). In certain embodiments, the substrate is a porous matrix. In certain embodiments, the matrix is agarose, cellulose, dextran, polyacrylamide, latex or controlled pore glass. In certain embodiments, the polypeptide described herein is immobilized onto a matrix (e.g., for affinity column).

### Certain Diagnostic Compositions

In certain embodiments, a polypeptide described herein is present in a composition that further comprises a carrier.

In certain embodiments, a polypeptide described herein is formulated for use in a diagnostic assay (e.g., immunoassay). In certain embodiments, the polypeptide is operably linked to a solid substrate or support, as described herein (e.g., a plate, chip, or particle). Accordingly, certain embodiments provide a diagnostic composition comprising a polypeptide described herein.

In certain embodiments, a polypeptide described herein is present in a lyophilized composition. In certain embodiments, the lyophilized composition further comprises one or more excipients selected from the group consisting of a cryo-lyoprotectant (e.g., trehalose, sucrose) and a bulking agent (e.g., mannitol, glycine).

“Antigen” refers to a molecule capable of being bound by an antibody. An antigen is additionally capable of being recognized by the immune system and/or being a target of a humoral immune response and/or cellular immune response. An antigen can have one or more epitopes (B- and/or T-cell epitopes).

The term, “antibody” or “immunoglobulin” is used herein in the broadest sense. “Antibody” refers to a polypeptide comprising an antigen binding region (including the complementarity determining region (CDRs)) from an immunoglobulin. An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa) connected by disulfide bonds. Each chain is composed of structural domains, which are referred to as immunoglobulin domains. These domains are classified into different categories by size and function, e.g., variable domains or regions on the light and heavy chains (V_{L} and V_{H}, respectively) and constant domains or regions on the light and heavy chains (C_{L} and C_{H}, respectively). The N-terminus of each chain defines a variable region of about 100 to 110 or more amino acids, referred to as the paratope, primarily responsible for antigen recognition, i.e., the antigen binding domain. Light chains are classified as either kappa or lambda. Heavy chains are classified as gamma, mu, alpha, delta, or epsilon, which in turn define the immunoglobulin classes, IgG, IgM, IgA, IgD and IgE, respectively. IgG antibodies are large molecules of about 150 kDa composed of four peptide chains. IgG antibodies contain two identical class y heavy chains of about 50 kDa and two identical light chains of about 25 kDa, thus a tetrameric quaternary structure. The two heavy chains are linked to each other and to a light chain each by disulfide bonds. The resulting tetramer has two identical halves, which together form the Y-like shape. Each end of the fork contains an identical antigen binding domain. There are four IgG subclasses (IgG1, IgG2, IgG3, and IgG4) in humans, named in order of their abundance in serum (i.e., IgG1 is the most abundant).

### Methods of Use

As described herein, a polypeptide described herein may be used for diagnostic assays.

### Diagnostic Methods

Certain embodiments of the invention provide a method of detecting a molecule associated with an immune response to SARS-CoV-2 or to a SARS-CoV-2 vaccine (e.g., an anti-SARS-CoV-2 antibody, or antigen specific T cell response) in an animal (e.g., human), the method comprising contacting a sample from the animal with a polypeptide or composition as described herein (e.g., detecting the presence or absence of an anti-SARS-CoV-2 antibody). In certain embodiments, the sample and the polypeptide are contacted under conditions suitable for the molecule (e.g., anti-SARS-CoV-2 antibody) to bind to the polypeptide. In certain embodiments, the method further comprises detecting the bound molecule. In certain embodiments, the immune response may be induced by natural infection of the virus, or by vaccination. In certain embodiments, the method further comprises measuring the level of the immune response. For example, in certain embodiments, the method comprises measuring anti-SARS-CoV-2 antibody titers in the sample. In certain embodiments, the sample is from an animal that has or had COVID-19. In certain embodiments, the sample is from an animal that is being tested to determine whether they had a SARS-CoV-2 infection. In certain embodiments, the sample is from an animal that was vaccinated against SARS-CoV-2.

Certain embodiments of the invention provide a method of detecting an anti-SARS-CoV-2 antibody in a test sample (e.g., a sample from an animal, such as a human), the method comprising contacting the test sample with a polypeptide or composition as described herein. In certain embodiments, the sample and the polypeptide are contacted under conditions suitable for an anti-SARS-CoV-2 antibody to bind to the polypeptide. In certain embodiments, the method further comprises detecting a bound anti-SARS-CoV-2 antibody. In certain embodiments, the method comprises measuring anti-SARS-CoV-2 antibody titers in the sample. In certain embodiments, the sample is from an animal that has or had COVID-19. In certain embodiments, the sample is from an animal that was vaccinated against SARS-CoV-2. In certain embodiments, the method further comprises diagnosing the animal as having or having had a SARS-CoV2 infection when anti-SARS-CoV-2 antibodies are detected, and optionally, further comprises administering an anti-SARS-CoV-2 therapeutic agent to the animal.

Certain embodiments of the invention provide a method of evaluating the immune response in an animal that received a SARS-CoV-2 vaccination, the method comprising contacting a test sample from the animal with a polypeptide or composition described herein and detecting the levels of anti-SARS-CoV2 antibodies in the test sample as compared to a control or reference value.

Certain embodiments of the invention also provide a method of identifying an animal that has or has had a SARS-CoV2 infection, the method comprising contacting a test sample from the animal with a polypeptide or composition described herein and detecting the presence or absence of an anti-SARS-CoV2 antibody in the test sample, wherein the animal is identified as having or having had a SARS-CoV2 infection when the presence of an anti-SARS-CoV2 antibody is detected.

Certain embodiments of the invention provide a method of diagnosing an animal as having or having had a SARS-CoV2 infection, the method comprising 1) obtaining a biological sample from the animal; 2) contacting the sample with a polypeptide or composition described herein and detecting whether anti-SARS-CoV-2 antibodies are present in the sample; and 3) diagnosing the animal as having or having had a SARS-CoV2 infection when anti-SARS-CoV-2 antibodies are detected.

In certain embodiments, the method describe herein further comprises detecting whether SARS-CoV-2 viral nucleic acid are present in a biological sample from the animal.

In certain embodiments, the method describe herein further comprises administering a therapeutic agent (e.g., an anti-SARS-CoV-2 therapeutic agent, such as an anti-viral agent such as Remdesivir, or an antibody therapy) to the animal.

A polypeptide described herein is readily applicable in antigen-antibody reactions with an antibody that is capable of binding to SARS-Cov-2 Spike protein. Thus, in certain embodiments, anti-SARS-CoV-2 antibodies are detected using an immunoassay, such as an immunoassay described herein. Typical immunoassay methods of antigen-antibody reactions include, but are not limited to, immunodiffusion assay, immunoelectrophoresis, agglutination assay, enzyme immunoassays, and radioimmunoassay (RIA).

In certain embodiments, the immunoassay method is a lateral flow assay.

In certain embodiments, the immunoassay method is an ELISA based method. In certain embodiments, the ELISA is an antigen-down ELISA. In certain embodiments, the ELISA is a capture assay also referred to as sandwich ELISA. In certain embodiments, the immunoassay method is a chemiluminescence based method.

In certain embodiments, the immunoassay method is conducted manually. In certain embodiments, the immunoassay method is conducted within an automated immunoassay system (e.g., a high throughput immunoassay automation system).

In certain embodiments, a test sample is contacted with the polypeptide or composition described herein for a first period. In certain embodiments, the polypeptide is immobilized on a solid substrate for a first period. In certain embodiments, the polypeptide described herein is directly immobilized on a solid substrate (e.g., adsorbed or chemically conjugated on a solid substrate). In certain embodiments, a polypeptide described herein is indirectly immobilized on a solid substrate via a capture agent (e.g., a capture antibody from the solid substrate in a sandwich ELISA). In certain embodiments, the polypeptide described herein is indirectly immobilized on a solid substrate via an anti-tag antibody. In certain embodiments, the polypeptide described herein is indirectly immobilized on an anti-tag antibody (e.g., anti-His tag antibody) pre-coated ELISA plate. In certain embodiments, the polypeptide described herein is indirectly immobilized on an anti-tag antibody (e.g., anti-His tag antibody) conjugated particle (e.g., magnetic particle), for example, in a chemiluminescence immunoassay.

In certain embodiments, the method comprises one or more washing steps after the first period.

In certain embodiments, the method comprises contacting the solid substrate with an enzyme-linked detection agent (e.g., alkaline phosphatase (ALP) or horseradish peroxidase (HRP) linked anti-IgG, anti-IgM or anti-IgA antibody) for a second period.

In certain embodiments, the method comprises one or more washing steps after the second period.

In certain embodiments, the method comprises contacting the solid substrate with a signal development agent (e.g., luminol, TMB, OPD) for a third period to produce detectable signal (e.g., luminescent light signal or chromogenic signal).

In certain embodiments, the test sample is a biological sample. In certain embodiment, the test sample is blood, plasma, serum, saliva, tears, feces and lavage (e.g., bronchoalveolar lavage). In certain embodiment, the test sample is serum.

In certain embodiments, the test sample is a clinically acquired biological sample. In certain embodiment, the test sample is a diluted biological sample. In certain embodiments, the test sample is a non-clinical research and development sample.

In certain embodiments, the anti-SARS-CoV-2 antibody is an IgM antibody. In certain embodiments, the anti-SARS-CoV-2 antibody is an IgA antibody. In certain embodiments, the anti-SARS-CoV-2 antibody is an IgG antibody. In certain embodiments, the anti-SARS-CoV-2 antibody is an IgG1 antibody. In certain embodiments, the anti-SARS-CoV-2 antibody is an IgG2 antibody. In certain embodiments, the anti-SARS-CoV-2 antibody is an IgG3 antibody. In certain embodiments, the anti-SARS-CoV-2 antibody is an IgG4 antibody.

In certain embodiments, SARS-CoV-2 specific T cell(s) may be detected in an ELISPOT assay using the engineered polypeptide described herein.

### Antibody Screening Methods

As described herein, certain embodiments of the invention provide a method of screening for a protective SARS-Cov-2 neutralizing agent using a polypeptide as described herein. In certain embodiments, the agent is an antibody or a fragment thereof. In certain embodiments, the agent is an aptamer. In certain embodiments, the agent is an affibody or nanobody.

Certain embodiments also provide a method for screening an anti-SARS-CoV-2 antibody, the method comprising screening a phage antibody/nanobody library against the polypeptide described herein (e.g., immobilized on a solid surface) and isolating the binder(s).

A polypeptide described herein may also be used to screen/characterize potential anti-SARS-CoV2 antibodies (e.g., for affinity or specificity). Accordingly, certain embodiments also provide a method for screening an antibody for affinity and/or specificity to SARS-CoV2, the method comprising contacting the antibody with a polypeptide described herein and measuring binding between the antibody and the polypeptide.

In certain embodiments, the binding strength or affinity is measured via an ELISA based assay or a SPR based assay.

Certain embodiments of the invention provide a method of screening for a SARS-Cov-2 neutralizing agent comprising contacting a polypeptide described herein with a display library (e.g., phage surface display, yeast surface display, ribosomal display) and selecting a binder(s) that has affinity or specificity for the polypeptide.

### Kits

Certain embodiments of the invention provide a kit comprising:
1) a polypeptide as described herein or a composition as described herein;
2) packaging material; and
3) instructions for using the polypeptide or composition, e.g., as described in a diagnostic method herein.

Certain embodiments of the invention provide a kit comprising:
1) a polypeptide described herein in a lyophilized formulation or a liquid formulation;
2) packaging material; and
3) instructions to reconstitute the polypeptide for use in a diagnostic method (e.g., a diagnostic as described herein).

In certain embodiments, the instruction further comprises directions for conjugating the polypeptide onto a substrate or other agent.

In certain embodiments, the kit further comprises optional components selected from the group consisting of an ELISA plate, a coating buffer, a blocking buffer, a diluent, a washing solution, a detection agent and a signal development agent as described above, a magnetic particle, a chromatography column, and an activating agent for conjugation to a fluorochrome.

Certain embodiments of the invention provide a kit comprising:
1) an ELISA plate coated with the polypeptide described herein;
2) packaging material; and
3) instructions to conduct an ELISA immunoassay.

In certain embodiments, the kit further comprises optional reagents selected from the group consisting of a blocking buffer, a diluent, a washing solution, a detection agent and signal development agent as described above.

Certain embodiments of the invention provide a kit comprising:
1) an ELISA plate coated with an anti-tag capture antibody (e.g., anti-His tag antibody);
2) a polypeptide as described herein or a composition as described herein;
3) packaging material; and
4) instructions to conduct an ELISA immunoassay.

Certain embodiments of the invention provide a kit comprising:
1) particles (e.g., polymeric particle or metal particle) conjugated with a polypeptide described herein;
2) packaging material; and
3) instructions to conduct an assay.

Certain embodiments of the invention provide a kit comprising:
1) lateral flow cartridge comprising a polypeptide described herein;
2) packaging material; and
3) instructions to conduct the lateral flow assay.

### Certain Polypeptides, Nucleic Acids, Vectors and Cells of the Invention

Certain embodiments of the invention provide a polypeptide comprising a SARS-CoV-2 spike ectodomain amino acid sequence, or a fragment thereof, wherein the amino acid sequence comprises R682A, R683G and R685G mutations, and optionally a D614G mutation. In certain embodiments, the polypeptide comprises the D614G mutation. In certain embodiments, the polypeptide further comprises one or more mutations that stabilize the spike ectodomain polypeptide. For example, in certain embodiments, the polypeptide comprises K986P and V987P mutations. In certain embodiments, the polypeptide described herein is a recombinant polypeptide. In certain embodiments, the polypeptide described herein is isolated or purified.

Thus, in certain embodiments, a polypeptide as described herein comprises R682A, R683G, and R685G mutations. In certain embodiments, a polypeptide as described herein comprises D614G, R682A, R683G, and R685G mutations. In certain embodiments, a polypeptide as described herein comprises R682A, R683G, R685G, K986P, and V987P mutations. In certain embodiments, a polypeptide as described herein comprises D614G, R682A, R683G, R685G, K986P, and V987P mutations.

In certain embodiments, the spike ectodomain amino acid sequence is between about 1,180 to about 1,230 amino acids in length. In certain embodiments, the spike ectodomain amino acid sequence is between about 1,185 to about 1,225 amino acids in length. In certain embodiments, the spike ectodomain amino acid sequence is between about 1,190 to about 1,220 amino acids in length. In certain embodiments, the spike ectodomain amino acid sequence is between about 1,192 to about 1,218 amino acids in length. In certain embodiments, the spike ectodomain amino acid sequence is between about 1,194 to about 1,216 amino acids in length. In certain embodiments, the spike ectodomain amino acid sequence is between about 1,194 to about 1,214 amino acids in length. In certain embodiments, the spike ectodomain amino acid sequence is between about 1,188 to about 1,208 amino acids in length. In certain embodiments, the spike ectodomain amino acid sequence is between about 1,190 to about 1,206 amino acids in length. In certain embodiments, the spike ectodomain amino acid sequence is between about 1,192 to about 1,204 amino acids in length. In certain embodiments, the spike ectodomain amino acid sequence is between about 1,194 to about 1,202 amino acids in length. In certain embodiments, the spike ectodomain amino acid sequence is between about 1,196 to about 1,200 amino acids in length. In certain embodiments, the spike ectodomain amino acid sequence is about 1,198 amino acids in length. In certain embodiments, the spike ectodomain amino acid sequence is between about 1,201 to about 1,221 amino acids in length. In certain embodiments, the spike ectodomain amino acid sequence is between about 1,203 to about 1,219 amino acids in length. In certain embodiments, the spike ectodomain amino acid sequence is between about 1,205 to about 1,217 amino acids in length. In certain embodiments, the spike ectodomain amino acid sequence is between about 1,207 to about 1,215 amino acids in length. In certain embodiments, the spike ectodomain amino acid sequence is between about 1,209 to about 1,213 amino acids in length. In certain embodiments, the spike ectodomain amino acid sequence is about 1,211 amino acids in length.

In certain embodiments, the SARS-CoV-2 spike ectodomain amino acid sequence comprises an amino acid sequence having at least about 80% sequence identity to SEQ ID NO:3 and comprises 1) R682A, R683G and R685G mutations, and optionally, 2) a D614G mutation; and/or 3) K986P, and V987P mutations. In certain embodiments, the SARS-CoV-2 spike ectodomain comprises an amino acid sequence having at least about 81%, 82%, 83%, 84%, 85%, 86%, 87%, 88%, 89%, 90%, 91%, 92%, 93%, 94%, 95%, 96%, 97%, 98%, 99% or 100% identity to SEQ ID NO:3; and comprises 1) R682A, R683G and R685G mutations, and optionally, 2) a D614G mutation; and/or 3) K986P, and V987P mutations. In certain embodiments, the SARS-CoV-2 spike ectodomain comprises an amino acid sequence having at least about 85% sequence identity to SEQ ID NO: 3; and comprises 1) R682A, R683G and R685G mutations, and optionally, 2) a D614G mutation; and/or 3) K986P, and V987P mutations. In certain embodiments, the SARS-CoV-2 spike ectodomain comprises an amino acid sequence having at least about 90% sequence identity to SEQ ID NO: 3; and comprises 1) R682A, R683G and R685G mutations, and optionally, 2) a D614G mutation; and/or 3) K986P, and V987P mutations. In certain embodiments, the SARS-CoV-2 spike ectodomain comprises an amino acid sequence having at least about 95% sequence identity to SEQ ID NO: 3; and comprises 1) R682A, R683G and R685G mutations, and optionally, 2) a D614G mutation; and/or 3) K986P, and V987P mutations.

In certain embodiments, the SARS-CoV-2 spike ectodomain amino acid sequence comprises an amino acid sequence having at least about 80% sequence identity to SEQ ID NO:3, and comprises 1) R682A, R683G and R685G mutations, and optionally, 2) a D614G mutation; and/or 3) K986P, and V987P mutations. In certain embodiments, the SARS-CoV-2 spike ectodomain amino acid sequence is further linked (e.g., through a peptide bond) to 1 to 20 (e.g., consecutive) amino acids provided in a sequence corresponding to SEQ ID NO:24. For example, in certain embodiments, the SARS-CoV-2 spike ectodomain carboxy-terminus is further linked to: ELGKYEQYIKWPWYIWLGFI (SEQ ID NO: 24), ELGKYEQYIKWPWYIWLGF (SEQ ID NO: 27), ELGKYEQYIKWPWYIWLG (SEQ ID NO: 28), ELGKYEQYIKWPWYIWL (SEQ ID NO: 29), ELGKYEQYIKWPWYIW (SEQ ID NO: 30), ELGKYEQYIKWPWYI (SEQ ID NO: 31), ELGKYEQYIKWPWY (SEQ ID NO: 32), ELGKYEQYIKWPW (SEQ ID NO: 33), ELGKYEQYIKWP (SEQ ID NO: 34), ELGKYEQYIKW (SEQ ID NO: 35), ELGKYEQYIK (SEQ ID NO: 36), ELGKYEQYI (SEQ ID NO: 37), ELGKYEQY (SEQ ID NO: 38), ELGKYEQ (SEQ ID NO: 39), ELGKYE (SEQ ID NO: 40), ELGKY (SEQ ID NO: 41), ELGK (SEQ ID NO: 42), ELG, EL, or E.

In certain embodiments, the SARS-CoV-2 spike ectodomain amino acid sequence comprises an amino acid sequence having at least about 80% sequence identity to SEQ ID NO:5-7. In certain embodiments, the SARS-CoV-2 spike ectodomain comprises an amino acid sequence having at least about 81%, 82%, 83%, 84%, 85%, 86%, 87%, 88%, 89%, 90%, 91%, 92%, 93%, 94%, 95%, 96%, 97%, 98%, 99% or 100% identity to SEQ ID NO:5-7. In certain embodiments, the SARS-CoV-2 spike ectodomain comprises an amino acid sequence having at least about 85% sequence identity to SEQ ID NO: 5-7. In certain embodiments, the SARS-CoV-2 spike ectodomain comprises an amino acid sequence having at least about 90% sequence identity to SEQ ID NO: 5-7. In certain embodiments, the SARS-CoV-2 spike ectodomain comprises an amino acid sequence having at least about 95% sequence identity to SEQ ID NO: 5-7. In certain embodiments, the SARS-CoV-2 spike ectodomain comprises an amino acid sequence having at least about 99% sequence identity to SEQ ID NO: 5-7. In certain embodiments, the SARS-CoV-2 spike ectodomain comprises SEQ ID NO: 5-7. In certain embodiments, the SARS-CoV-2 spike ectodomain consists of SEQ ID NO: 5-7.

In certain embodiments, the SARS-CoV-2 spike ectodomain comprises an amino acid sequence having at least about 80% sequence identity to SEQ ID NO:5. In certain embodiments, the SARS-CoV-2 spike ectodomain comprises an amino acid sequence having at least about 81%, 82%, 83%, 84%, 85%, 86%, 87%, 88%, 89%, 90%, 91%, 92%, 93%, 94%, 95%, 96%, 97%, 98%, 99% or 100% identity to SEQ ID NO:5. In certain embodiments, the SARS-CoV-2 spike ectodomain comprises an amino acid sequence having at least about 85% sequence identity to SEQ ID NO:5. In certain embodiments, the SARS-CoV-2 spike ectodomain comprises an amino acid sequence having at least about 90% sequence identity to SEQ ID NO:5. In certain embodiments, the SARS-CoV-2 spike ectodomain comprises an amino acid sequence having at least about 95% sequence identity to SEQ ID NO:5. In certain embodiments, the SARS-CoV-2 spike ectodomain comprises an amino acid sequence having at least about 99% sequence identity to SEQ ID NO:5. In certain embodiments, the SARS-CoV-2 spike ectodomain comprises SEQ ID NO:5. In certain embodiments, the SARS-CoV-2 spike ectodomain consists of SEQ ID NO:5.

In certain embodiments, the SARS-CoV-2 spike ectodomain comprises an amino acid sequence having at least about 80% sequence identity to SEQ ID NO:6. In certain embodiments, the SARS-CoV-2 spike ectodomain comprises an amino acid sequence having at least about 81%, 82%, 83%, 84%, 85%, 86%, 87%, 88%, 89%, 90%, 91%, 92%, 93%, 94%, 95%, 96%, 97%, 98%, 99% or 100% identity to SEQ ID NO:6. In certain embodiments, the SARS-CoV-2 spike ectodomain comprises an amino acid sequence having at least about 85% sequence identity to SEQ ID NO:6. In certain embodiments, the SARS-CoV-2 spike ectodomain comprises an amino acid sequence having at least about 90% sequence identity to SEQ ID NO:6. In certain embodiments, the SARS-CoV-2 spike ectodomain comprises an amino acid sequence having at least about 95% sequence identity to SEQ ID NO:6. In certain embodiments, the SARS-CoV-2 spike ectodomain comprises an amino acid sequence having at least about 99% sequence identity to SEQ ID NO:6. In certain embodiments, the SARS-CoV-2 spike ectodomain comprises SEQ ID NO:6. In certain embodiments, the SARS-CoV-2 spike ectodomain consists of SEQ ID NO:6.

In certain embodiments, the SARS-CoV-2 spike ectodomain comprises an amino acid sequence having at least about 80% sequence identity to SEQ ID NO:7. In certain embodiments, the SARS-CoV-2 spike ectodomain comprises an amino acid sequence having at least about 81%, 82%, 83%, 84%, 85%, 86%, 87%, 88%, 89%, 90%, 91%, 92%, 93%, 94%, 95%, 96%, 97%, 98%, 99% or 100% identity to SEQ ID NO:7. In certain embodiments, the SARS-CoV-2 spike ectodomain comprises an amino acid sequence having at least about 85% sequence identity to SEQ ID NO:7. In certain embodiments, the SARS-CoV-2 spike ectodomain comprises an amino acid sequence having at least about 90% sequence identity to SEQ ID NO:7. In certain embodiments, the SARS-CoV-2 spike ectodomain comprises an amino acid sequence having at least about 95% sequence identity to SEQ ID NO:7. In certain embodiments, the SARS-CoV-2 spike ectodomain comprises an amino acid sequence having at least about 99% sequence identity to SEQ ID NO:7. In certain embodiments, the SARS-CoV-2 spike ectodomain comprises SEQ ID NO:7. In certain embodiments, the SARS-CoV-2 spike ectodomain consists of SEQ ID NO:7.

In certain embodiments, a polypeptide as described herein comprises a trimerization motif, such as a foldon trimer motif or a GCN4 motif. Accordingly, a polypeptide described herein comprising a SARS-CoV-2 spike ectodomain may assemble into a trimer, thereby providing a timeric protein. In certain embodiments, the polypeptide described herein is operably linked to a trimerization motif. For example, a trimerization motif may be fused either directly or indirectly via a linker group to the N-terminus or C-terminus of a spike ectodomain amino acid sequence described herein (e.g., fused to the C-terminus). In certain embodiments, the trimerization motif is a foldon trimer motif (e.g., bacteriophage T4 fibritin foldon trimer motif). In certain embodiments, the foldon trimer motif comprises/consists of SEQ ID NO: 16. In certain embodiments, the trimerization motif is a GCN4 motif. In certain embodiments, the GCN4 motif comprises/consists of SEQ ID NO: 17.

In certain embodiments, a spike ectodomain amino acid sequence described herein may be directly linked to a trimerization motif. In certain embodiments, a spike ectodomain amino acid sequence described herein may be operably linked to a trimerization motif via a linker group, such as a peptide linker. While the linker group may vary, it should not interfere with the function of the spike ectodomain or the trimerization motif. For example, the linker can be a flexible peptide linker such as a glycine rich linker. In certain embodiments, the linker is a GS rich amino acid sequence. In certain embodiments, the linker is a di-peptide, such as GS. In certain embodiments, the linker is a GSG (SEQ ID NO: 15).

In certain embodiments, the linker group is about 1 to about 30 amino acids in length. In certain embodiments, the linker group is about 1 to about 25 amino acids in length. In certain embodiments, the linker group is about 1 to about 20 amino acids in length. In certain embodiments, the linker group is about 1 to about 15 amino acids in length. In certain embodiments, the linker group is about 1 to about 12 amino acids in length. In certain embodiments, the linker group is about 1 to about 10 amino acids in length. In certain embodiments, the linker group is about 1 to about 9 amino acids in length. In certain embodiments, the linker group is about 1 to about 8 amino acids in length. In certain embodiments, the linker group is about 1 to about 7 amino acids in length. In certain embodiments, the linker group is about 1 to about 6 amino acids in length. In certain embodiments, the linker group is about 1 to about 5 amino acids in length. In certain embodiments, the linker group is about 1 to about 4 amino acids in length. In certain embodiments, the linker group is about 1 to about 3 amino acids in length. In certain embodiments, the linker group is about 2 amino acids in length. In certain embodiments, the linker group is about 1 amino acid in length.

In certain embodiments, a polypeptide as described herein may be further operably linked (e.g., either directly or through a linker group) to one or more detectable markers, such as a protein or peptide tag or a fluorescent tag. In certain embodiments, a polypeptide as described herein may be further operably linked (e.g., directly or through a linker group) to one or more protein or peptide tags that are useful for purification (e.g., an epitope tag or an affinity tag).

In certain embodiments, a polypeptide as described herein is operably linked to a protein or peptide tag. In certain embodiments, the tag is a poly(His) tag, FLAG, 3×FLAG, c-Myc, Fc tag or a hemagglutinin tag (e.g. HA). In certain embodiments, the tag is a poly(His) tag. In certain embodiments, the peptide tag is a 6×His tag (SEQ ID NO: 20). In certain embodiments, the tag is an 8×His tag (SEQ ID NO: 43).

In some embodiments, the protein or peptide tag is operably linked to the N-terminal end and/or the C-terminal end of the polypeptide described herein (e.g., to the N- or C-terminus of the spike ectodomain amino acid sequence, such as the C-terminus).

In certain embodiments, the polypeptide (e.g., the spike ectodomain amino acid sequence) is directly linked to a protein or peptide tag. In certain embodiments, the polypeptide (e.g., the spike ectodomain amino acid sequence) is linked to a protein or peptide tag via a linker group. While the linker group may vary, it should not interfere with the function of the polypeptide (e.g., the spike ectodomain). For example, the linker can be a flexible peptide linker such as a glycine rich linker. In certain embodiments, the linker is a GS rich amino acid sequence. In certain embodiments, the linker is a di-peptide, such as GS. In certain embodiments, the linker is a GSG (SEQ ID NO: 15). In certain embodiments, the linker group is a single Glycine. In certain embodiments, the linker group comprises a trimerization motif described herein. For example, in certain embodiments, the linker comprises/consists of SEQ ID NO: 18 or 19.

In certain embodiments, the linker group is about 1 to about 50 amino acids in length. In certain embodiments, the linker group is about 1 to about 45 amino acids in length. In certain embodiments, the linker group is about 1 to about 40 amino acids in length. In certain embodiments, the linker group is about 1 to about 35 amino acids in length. In certain embodiments, the linker group is about 1 to about 30 amino acids in length. In certain embodiments, the linker group is about 1 to about 25 amino acids in length. In certain embodiments, the linker group is about 1 to about 20 amino acids in length. In certain embodiments, the linker group is about 1 to about 15 amino acids in length. In certain embodiments, the linker group is about 1 to about 12 amino acids in length. In certain embodiments, the linker group is about 1 to about 10 amino acids in length. In certain embodiments, the linker group is about 1 to about 9 amino acids in length. In certain embodiments, the linker group is about 1 to about 8 amino acids in length. In certain embodiments, the linker group is about 1 to about 7 amino acids in length. In certain embodiments, the linker group is about 1 to about 6 amino acids in length. In certain embodiments, the linker group is about 1 to about 5 amino acids in length. In certain embodiments, the linker group is about 1 to about 4 amino acids in length. In certain embodiments, the linker group is about 1 to about 3 amino acids in length. In certain embodiments, the linker group is about 2 amino acids in length. In certain embodiments, the linker group is about 1 amino acid in length. In certain embodiments, the linker group is about 30 amino acids in length. In certain embodiments, the linker group is about 32 amino acids in length.

In certain embodiments, the polypeptide comprises an amino acid sequence having at least about 80% sequence identity to SEQ ID NO:8, 10, 12, or 25. In certain embodiments, the polypeptide comprises an amino acid sequence having at least about 81%, 82%, 83%, 84%, 85%, 86%, 87%, 88%, 89%, 90%, 91%, 92%, 93%, 94%, 95%, 96%, 97%, 98%, 99% or 100% identity to SEQ ID NO: 8, 10, 12, or 25. In certain embodiments, the polypeptide comprises an amino acid sequence having at least about 85% sequence identity to SEQ ID NO: 8, 10, 12, or 25. In certain embodiments, the polypeptide comprises an amino acid sequence having at least about 90% sequence identity to SEQ ID NO: 8, 10, 12, or 25. In certain embodiments, the polypeptide comprises an amino acid sequence having at least about 95% sequence identity to SEQ ID NO: 8, 10, 12, or 25. In certain embodiments, the polypeptide comprises an amino acid sequence having at least about 99% sequence identity to SEQ ID NO: 8, 10, 12, or 25. In certain embodiments, the polypeptide comprises SEQ ID NO: 8, 10, 12, or 25. In certain embodiments, the polypeptide consists of SEQ ID NO: 8, 10, 12, or 25.

In certain embodiments, the polypeptide comprises an amino acid sequence having at least about 80% sequence identity to SEQ ID NO:8. In certain embodiments, the polypeptide comprises an amino acid sequence having at least about 81%, 82%, 83%, 84%, 85%, 86%, 87%, 88%, 89%, 90%, 91%, 92%, 93%, 94%, 95%, 96%, 97%, 98%, 99% or 100% identity to SEQ ID NO:8. In certain embodiments, the polypeptide comprises an amino acid sequence having at least about 85% sequence identity to SEQ ID NO:8. In certain embodiments, the polypeptide comprises an amino acid sequence having at least about 90% sequence identity to SEQ ID NO:8. In certain embodiments, the polypeptide comprises an amino acid sequence having at least about 95% sequence identity to SEQ ID NO:8. In certain embodiments, the polypeptide comprises an amino acid sequence having at least about 99% sequence identity to SEQ ID NO:8. In certain embodiments, the polypeptide comprises SEQ ID NO:8. In certain embodiments, the polypeptide consists of SEQ ID NO:8.

In certain embodiments, the polypeptide comprises an amino acid sequence having at least about 80% sequence identity to SEQ ID NO:10. In certain embodiments, the polypeptide comprises an amino acid sequence having at least about 81%, 82%, 83%, 84%, 85%, 86%, 87%, 88%, 89%, 90%, 91%, 92%, 93%, 94%, 95%, 96%, 97%, 98%, 99% or 100% identity to SEQ ID NO:10. In certain embodiments, the polypeptide comprises an amino acid sequence having at least about 85% sequence identity to SEQ ID NO:10. In certain embodiments, the polypeptide comprises an amino acid sequence having at least about 90% sequence identity to SEQ ID NO:10. In certain embodiments, the polypeptide comprises an amino acid sequence having at least about 95% sequence identity to SEQ ID NO:10. In certain embodiments, the polypeptide comprises an amino acid sequence having at least about 99% sequence identity to SEQ ID NO:10. In certain embodiments, the polypeptide comprises SEQ ID NO:10. In certain embodiments, the polypeptide consists of SEQ ID NO:10.

In certain embodiments, the polypeptide comprises an amino acid sequence having at least about 80% sequence identity to SEQ ID NO:12. In certain embodiments, the polypeptide comprises an amino acid sequence having at least about 81%, 82%, 83%, 84%, 85%, 86%, 87%, 88%, 89%, 90%, 91%, 92%, 93%, 94%, 95%, 96%, 97%, 98%, 99% or 100% identity to SEQ ID NO:12. In certain embodiments, the polypeptide comprises an amino acid sequence having at least about 85% sequence identity to SEQ ID NO:12. In certain embodiments, the polypeptide comprises an amino acid sequence having at least about 90% sequence identity to SEQ ID NO:12. In certain embodiments, the polypeptide comprises an amino acid sequence having at least about 95% sequence identity to SEQ ID NO:12. In certain embodiments, the polypeptide comprises an amino acid sequence having at least about 99% sequence identity to SEQ ID NO:12. In certain embodiments, the polypeptide comprises SEQ ID NO:12. In certain embodiments, the polypeptide consists of SEQ ID NO:12.

In certain embodiments, the polypeptide comprises an amino acid sequence having at least about 80% sequence identity to SEQ ID NO:25. In certain embodiments, the polypeptide comprises an amino acid sequence having at least about 81%, 82%, 83%, 84%, 85%, 86%, 87%, 88%, 89%, 90%, 91%, 92%, 93%, 94%, 95%, 96%, 97%, 98%, 99% or 100% identity to SEQ ID NO:25. In certain embodiments, the polypeptide comprises an amino acid sequence having at least about 85% sequence identity to SEQ ID NO:25. In certain embodiments, the polypeptide comprises an amino acid sequence having at least about 90% sequence identity to SEQ ID NO:25. In certain embodiments, the polypeptide comprises an amino acid sequence having at least about 95% sequence identity to SEQ ID NO:25. In certain embodiments, the polypeptide comprises an amino acid sequence having at least about 99% sequence identity to SEQ ID NO:25. In certain embodiments, the polypeptide comprises SEQ ID NO:25. In certain embodiments, the polypeptide consists of SEQ ID NO:25.

In certain embodiments, the polypeptide does not comprise a spike transmembrane segment. In certain embodiments, the polypeptide does not comprise a spike intracellular segment. In certain embodiments, the polypeptide does not comprise a spike transmembrane segment or a spike intracellular segment. In certain embodiments, the polypeptide is a SAR-COV-2 spike ectodomain polypeptide as described herein that is optionally operably linked to a peptide tag (e.g., directly or through a linker group described herein, such as a linker group comprising a trimerization motif). In certain embodiments, the polypeptide consists of a spike ectodomain polypeptide described herein. In certain embodiments, the polypeptide consists of a spike ectodomain polypeptide as described herein and a trimerization motif. In certain embodiments, the polypeptide consists of a spike ectodomain polypeptide as described herein, a linker group, and a trimerization motif. In certain embodiments, the polypeptide consists of a spike ectodomain polypeptide as described herein, an optional linker group (e.g., comprising a trimerization motif), and a peptide tag.

In certain embodiments, the polypeptide further comprises a signal peptide. In certain embodiments, the signal peptide is a wildtype SARS-CoV-2 spike protein signal peptide (e.g., SEQ ID NO: 14). In certain embodiments, the signal peptide is cleaved from the polypeptide prior to secretion.

In certain embodiments, the signal peptide is operably linked to the N-terminus of the spike ectodomain amino acid sequence. In certain embodiments, the signal peptide is operably linked to the C-terminus of the spike ectodomain amino acid sequence.

In certain embodiments, the signal peptide is directly linked to the spike ectodomain amino acid sequence. In certain embodiments, the signal peptide is linked to the spike ectodomain amino acid sequence through a linker group. In certain embodiments, the linker group is a peptide linker.

In certain embodiments, the polypeptide comprises an amino acid sequence having at least about 80% identity to SEQ ID NO:9. In certain embodiments, the polypeptide comprises an amino acid sequence having at least about 81%, 82%, 83%, 84%, 85%, 86%, 87%, 88%, 89%, 90%, 91%, 92%, 93%, 94%, 95%, 96%, 97%, 98%, 99% or 100% identity to SEQ ID NO:9. In certain embodiments, the polypeptide comprises an amino acid sequence having at least about 85% sequence identity to SEQ ID NO:9. In certain embodiments, the polypeptide comprises an amino acid sequence having at least about 90% sequence identity to SEQ ID NO:9. In certain embodiments, the polypeptide comprises an amino acid sequence having at least about 95% sequence identity to SEQ ID NO:9. In certain embodiments, the polypeptide comprises an amino acid sequence having at least about 99% sequence identity to SEQ ID NO:9. In certain embodiments, the polypeptide comprises SEQ ID NO:9. In certain embodiments, the polypeptide consists of SEQ ID NO:9.

In certain embodiments, the polypeptide comprises an amino acid sequence having at least about 80% identity to SEQ ID NO:11. In certain embodiments, the polypeptide comprises an amino acid sequence having at least about 81%, 82%, 83%, 84%, 85%, 86%, 87%, 88%, 89%, 90%, 91%, 92%, 93%, 94%, 95%, 96%, 97%, 98%, 99% or 100% identity to SEQ ID NO:11. In certain embodiments, the polypeptide comprises an amino acid sequence having at least about 85% sequence identity to SEQ ID NO:11. In certain embodiments, the polypeptide comprises an amino acid sequence having at least about 90% sequence identity to SEQ ID NO:11. In certain embodiments, the polypeptide comprises an amino acid sequence having at least about 95% sequence identity to SEQ ID NO:11. In certain embodiments, the polypeptide comprises an amino acid sequence having at least about 99% sequence identity to SEQ ID NO:11. In certain embodiments, the polypeptide comprises SEQ ID NO:11. In certain embodiments, the polypeptide consists of SEQ ID NO:11.

In certain embodiments, the polypeptide comprises an amino acid sequence having at least about 80% identity to SEQ ID NO:13. In certain embodiments, the polypeptide comprises an amino acid sequence having at least about 81%, 82%, 83%, 84%, 85%, 86%, 87%, 88%, 89%, 90%, 91%, 92%, 93%, 94%, 95%, 96%, 97%, 98%, 99% or 100% identity to SEQ ID NO:13. In certain embodiments, the polypeptide comprises an amino acid sequence having at least about 85% sequence identity to SEQ ID NO:13. In certain embodiments, the polypeptide comprises an amino acid sequence having at least about 90% sequence identity to SEQ ID NO:13. In certain embodiments, the polypeptide comprises an amino acid sequence having at least about 95% sequence identity to SEQ ID NO:13. In certain embodiments, the polypeptide comprises an amino acid sequence having at least about 99% sequence identity to SEQ ID NO:13. In certain embodiments, the polypeptide comprises SEQ ID NO:13. In certain embodiments, the polypeptide consists of SEQ ID NO:13.

In certain embodiments, the polypeptide comprises an amino acid sequence having at least about 80% identity to SEQ ID NO:26. In certain embodiments, the polypeptide comprises an amino acid sequence having at least about 81%, 82%, 83%, 84%, 85%, 86%, 87%, 88%, 89%, 90%, 91%, 92%, 93%, 94%, 95%, 96%, 97%, 98%, 99% or 100% identity to SEQ ID NO: 26. In certain embodiments, the polypeptide comprises an amino acid sequence having at least about 85% sequence identity to SEQ ID NO:26. In certain embodiments, the polypeptide comprises an amino acid sequence having at least about 90% sequence identity to SEQ ID NO:26. In certain embodiments, the polypeptide comprises an amino acid sequence having at least about 95% sequence identity to SEQ ID NO:26. In certain embodiments, the polypeptide comprises an amino acid sequence having at least about 99% sequence identity to SEQ ID NO:13. In certain embodiments, the polypeptide comprises SEQ ID NO:26. In certain embodiments, the polypeptide consists of SEQ ID NO:26.

In certain embodiments, the polypeptide is between about 1,180 to about 3,000 amino acids in length. In certain embodiments, the polypeptide is between about 1,180 to about 2,500 amino acids in length. In certain embodiments, the polypeptide is between about 1,180 to about 2,225 amino acids in length. In certain embodiments, the polypeptide is between about 1,180 to about 2,000 amino acids in length. In certain embodiments, the polypeptide is between about 1,180 to about 1,750 amino acids in length. In certain embodiments, the polypeptide is between about 1,180 to about 1,500 amino acids in length. In certain embodiments, the polypeptide is between about 1,180 to about 1,400 amino acids in length. In certain embodiments, the polypeptide is between about 1,180 to about 1,300 amino acids in length. In certain embodiments, the polypeptide is between about 1,180 to about 1,275 amino acids in length. In certain embodiments, the polypeptide is between about 1,180 to about 1,263 amino acids in length. In certain embodiments, the polypeptide is between about 1,190 to about 1,263 amino acids in length. In certain embodiments, the polypeptide is between about 1,200 to about 1,263 amino acids in length. In certain embodiments, the polypeptide is between about 1,210 to about 1,263 amino acids in length. In certain embodiments, the polypeptide is between about 1,220 to about 1,263 amino acids in length. In certain embodiments, the polypeptide is between about 1,225 to about 1,263 amino acids in length. In certain embodiments, the polypeptide is between about 1,230 to about 1,257 amino acids in length. In certain embodiments, the polypeptide is between about 1,248 to about 1,258 amino acids in length. In certain embodiments, the polypeptide is between about 1,251 to about 1,255 amino acids in length. In certain embodiments, the polypeptide is about 1,253 amino acids in length. In certain embodiments, the polypeptide is between about 1,242 to about 1,252 amino acids in length. In certain embodiments, the polypeptide is between about 1,245 to about 1,249 amino acids in length. In certain embodiments, the polypeptide is about 1,247 amino acids in length. In certain embodiments, the polypeptide is between about 1,235 to about 1,245 amino acids in length. In certain embodiments, the polypeptide is between about 1,238 to about 1,242 amino acids in length. In certain embodiments, the polypeptide is about 1,240 amino acids in length. In certain embodiments, the polypeptide is between about 1,229 to about 1,239 amino acids in length. In certain embodiments, the polypeptide is between about 1,232 to about 1,236 amino acids in length. In certain embodiments, the polypeptide is about 1,234 amino acids in length.

In certain embodiments, the polypeptide is capable of binding to angiotensin-converting enzyme 2 (ACE2) (e.g., with high affinity). In certain embodiments, ACE2 is human ACE2.

In certain embodiments, the polypeptide may be operably linked to a solid substrate as described herein (e.g., immobilized on a solid substrate).

Certain embodiments of the invention provide a polypeptide as described herein, which comprises R682A, R683G, and R685G mutations, and optionally a D614G mutation, in the spike ectodomain.

Certain embodiments of the invention provide a polypeptide produced using a method described herein, wherein the polypeptide comprises R682A, R683G, and R685G mutations, and optionally a D614G mutation, in the spike ectodomain.

Certain embodiments of the invention provide a polypeptide produced from a cell described herein (e.g., a stably transfected mammalian cell), wherein the polypeptide comprises R682A, R683G, and R685G mutations, and optionally a D614G mutation, in the spike ectodomain.

Certain embodiments also provide a composition comprising a polypeptide described herein and a carrier, wherein the polypeptide comprises R682A, R683G, and R685G mutations, and optionally a D614G mutation, in the spike ectodomain. In certain embodiments, the composition is a diagnostic composition described herein. In certain embodiments, a polypeptide described herein is present in a lyophilized composition. In certain embodiments, the lyophilized composition further comprises one or more excipients selected from the group consisting of a cryo-lyoprotectant (e.g., trehalose, sucrose) and a bulking agent (e.g., mannitol, glycine).

Certain embodiments also provide a kit comprising a polypeptide as described herein and instructions for use, wherein the polypeptide comprises R682A, R683G, and R685G mutations, and optionally a D614G mutation, in the spike ectodomain.

### Nucleic Acids, Expression Cassettes and Vectors

Certain embodiments provide polynucleotides (e.g., isolated polynucleotides) comprising a nucleic acid sequence encoding a polypeptide described herein, wherein the polypeptide comprises R682A, R683G, and R685G mutations, and optionally a D614G mutation, in the spike ectodomain. The polynucleotides may be single-stranded or double-stranded. In some embodiments, the polynucleotide is DNA. In some embodiments, the polynucleotide is cDNA. In some embodiments, the polynucleotide is RNA. In some embodiments, the polynucleotide comprises a nucleic acid sequence that has at least about 90% (e.g., at least about 90%, 91%, 92%, 93%, 94%, 95%, 96%, 97%, 98%, 99% or 100%) sequence identity to SEQ ID NO:22 or 23.

In certain embodiments, the nucleic acid further comprises a promoter.

Certain embodiments of the invention provide an expression cassette comprising a nucleic acid sequence described herein and a promoter operably linked to the nucleic acid.

In certain embodiments, the promoter is a regulatable promoter. In certain embodiments, the promoter is a constitutive promoter.

In certain embodiments, the expression cassette further comprises an expression control sequence (e.g., an enhancer) operably linked to the nucleic acid sequence. Expression control sequences and techniques for operably linking sequences together are well known in the art.

Nucleic acids/expression cassettes encoding a polypeptide described herein can be engineered into a vector using standard ligation techniques, such as those described in Sambrook and Russell, Molecular Cloning: A Laboratory Manual, Cold Spring Harbor Laboratory Press Cold Spring Harbor, N.Y. (2001). For example, ligations can be accomplished in 20 mM Tris-Cl pH 7.5, 10 mM MgCl2, 10 mM DTT, 33 μg/ml BSA, 10 mM-50 mM NaCl, and either 40 μM ATP, 0.01-0.02 (Weiss) units T4 DNA ligase at 0° C. (for “sticky end” ligation) or 1 mM ATP, 0.3-0.6 (Weiss) units T4 DNA ligase at 14° C. (for “blunt end” ligation). Intermolecular “sticky end” ligations are usually performed at 30-100 μg/ml total DNA concentrations (5-100 nM total end concentration).

Accordingly, certain embodiments of the invention provide a vector comprising an expression cassette described herein. In particular, certain embodiments provide a vector comprising an expression cassette comprising a promoter operably linked to a nucleic acid sequence encoding a polypeptide of the invention (e.g., SEQ ID NO:5-13 or 25-26). In certain embodiments, the promoter is a CMV promoter.

Non-limiting examples of vectors include plasmids and viral expression systems, such as a lentiviral, adenoviral, and adeno-associated virus (AAV) expression systems. Further non-limiting examples mammalian expression vectors include the pRc/CMV, pSV2gpt, pSV2neo, pcDNA3, pcDNAI/amp, pcDNAI/neo, pSV2-dhfr, pMSG, pSVT7, pTk2, pRSVneo, pko-neo, and pHyg-derived vectors. In certain embodiments, the vector is a lentivirus vector. In certain embodiments, the vector is a vector described herein.

### Cells

Certain embodiments of the invention provide a cell comprising a polypeptide described herein, a nucleic acid described herein, an expression cassette described herein or a vector described herein, wherein the polypeptide comprises R682A, R683G, and R685G mutations, and optionally a D614G mutation, in the spike ectodomain or the nucleic acid/expression cassette or vector encodes such a polypeptide.

In certain embodiments, the cell is a mammalian cell. In certain embodiments, the cell is stably transfected. In certain embodiments, the cell is stably transduced.

In certain embodiments, the cell is a human mammalian cell. In certain embodiments, the cell is a human embryonic kidney (HEK) 293 cell. In certain embodiments, the cell is a 293F cell. In certain embodiments, the cell is a 293T cell. In certain embodiments, the cell is a human embryonic retinal (PER.C6) cell. In certain embodiments, the cell is a HT-1080 cell. In certain embodiments, the cell is a Huh-7 cell.

In certain embodiments, the cell is a non-human mammalian cell. In certain embodiments, the cell is a Monkey kidney epithelial (Vero) cell. In certain embodiments, the cell is a Chinese Hamster Ovary (CHO) cell. In certain embodiments, the cell is a baby hamster kidney (BHK) cell.

In certain embodiments, the cell is a non-mammalian cell. In certain embodiments, the cell is an insect cell. In certain embodiments, the cell is a yeast cell. In certain embodiments, the cell is a bacteria cell.

Certain embodiments provide a cell produced using a method described herein.

### Methods of Producing a Cell or Recombinant Polypeptide of the Invention

Certain embodiments of the invention provide a method of making a genetically modified cell capable of producing a polypeptide described herein, the method comprising transfecting or transducing a cell with a nucleic acid, expression cassette or vector described herein, wherein the polypeptide comprises R682A, R683G, and R685G mutations, and optionally a D614G mutation, in the spike ectodomain or the nucleic acid/expression cassette or vector encodes such a polypeptide. In certain embodiments, the vector comprises a selectable marker. In certain embodiments, the vector is a lentivirus vector.

Certain embodiments also provide a method of producing a polypeptide described herein (i.e., comprising R682A, R683G, and R685G mutations, and optionally a D614G mutation, in the spike ectodomain), comprising transfecting or transducing a cell with a nucleic acid, expression cassette or vector described herein. In certain embodiments, the vector comprises a selectable marker. In certain embodiments, the vector is a lentivirus vector.

In certain embodiments, the cell is a mammalian cell. In certain embodiments, the cell is a 293F cell. In certain embodiments, the cell is a cell described herein.

In certain embodiments, the method further comprises culturing the cell under appropriate conditions and for a sufficient time to allow expression of the recombinant polypeptide to occur. In certain embodiments, the cell is cultured for about 12 h, 18 h, 24 h, 36 h, 48 h, 3 days, 4 days, 5 days, 6 days, 1 week, 2 weeks, 3 weeks, 4 weeks or more.

In some embodiments, a selectable marker is used to select cells that have been successfully transfected/transduced. As a non-limiting example, antibiotic resistance (e.g., to puromycin or another antibiotic) can be used to select genetically modified cells that contain an expression construct of interest.

In certain embodiments, the cell is stably transfected. In certain embodiments, the cell is stably transduced.

In certain embodiments, the method further comprises isolating the recombinant polypeptide from the cell, cellular components and/or the growth media.

In certain embodiments, the cell secretes the recombinant polypeptide into the cell growth media. In certain embodiments, the recombinant polypeptide is isolated from the growth media.

In certain embodiments, the method further comprises purifying the isolated recombinant polypeptide. In certain embodiments, the recombinant polypeptide is purified using an affinity column (e.g., a Nickel affinity column). In certain embodiments, the recombinant polypeptide is purified using gel filtration. In certain embodiments, the recombinant polypeptide is purified using an ion exchange column.

In certain embodiments, isolated recombinant polypeptide is substantially pure. For example, in certain embodiments, the isolated recombinant polypeptide comprises less than about 30% contaminants, such as host cell proteins or growth media. In certain embodiments, the isolated recombinant polypeptide comprises less than about 25%, 20%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, 0.5%, 0.4%, 0.3%, 0.2%, 0.1% or less of contaminants, such as host cell proteins or growth media.

In certain embodiments, a method described herein may be used to produce a polypeptide as described herein 1) that is biologically active (e.g., capable of binding to ACE2 with, e.g., high affinity and specificity) and/or 2) which is properly folded. Thus, in certain embodiments, the produced polypeptide is capable of binding to ACE2. In certain embodiments, the produced polypeptide is properly folded.

In certain embodiments, a method described herein may be used to produce a high yield of a polypeptide of the invention that has enhanced stability and/or resistance against denaturation or proteolytic degradation.

### Certain Definitions

The term “nucleic acid” refers to deoxyribonucleotides or ribonucleotides and polymers thereof in either single- or double-stranded form, composed of monomers (nucleotides) containing a sugar, phosphate and a base which is either a purine or pyrimidine. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al. (1991) Nucl. Acids Res., 19:508; Ohtsuka et al. (1985) JBC, 260:2605; Rossolini et al. (1994) Mol. Cell. Probes, 8:91. A “nucleic acid fragment” is a fraction of a given nucleic acid molecule. Deoxyribonucleic acid (DNA) in the majority of organisms is the genetic material while ribonucleic acid (RNA) is involved in the transfer of information contained within DNA into proteins. The term “nucleotide sequence” refers to a polymer of DNA or RNA that can be single- or double-stranded, optionally containing synthetic, non-natural or altered nucleotide bases capable of incorporation into DNA or RNA polymers. The terms “nucleic acid,” “nucleic acid molecule,” “nucleic acid fragment,” “nucleic acid sequence or segment,” or “polynucleotide” may also be used interchangeably with gene, cDNA, DNA and RNA encoded by a gene.

By “portion” or “fragment,” as it relates to a nucleic acid molecule, sequence or segment of the invention, when it is linked to other sequences for expression, is meant a sequence having at least 80 nucleotides, more preferably at least 150 nucleotides, and still more preferably at least 400 nucleotides. If not employed for expressing, a “portion” or “fragment” means at least 9, preferably 12, more preferably 15, even more preferably at least 20, consecutive nucleotides, e.g., probes and primers (oligonucleotides), corresponding to the nucleotide sequence of the nucleic acid molecules of the invention.

The term “amino acid,” comprises the residues of the natural amino acids (e.g. Ala, Arg, Asn, Asp, Cys, Glu, Gln, Gly, His, Hyl, Hyp, Ile, Leu, Lys, Met, Phe, Pro, Ser, Thr, Trp, Tyr, and Val) in D or L form, as well as unnatural amino acids (e.g. phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine, ornithine, citruline, α-methyl-alanine, para-benzoylphenylalanine, phenylglycine, propargylglycine, sarcosine, and tert-butylglycine). The term also comprises natural and unnatural amino acids bearing a conventional amino protecting group (e.g. acetyl or benzyloxycarbonyl), as well as natural and unnatural amino acids protected at the carboxy terminus (e.g. as a (C₁-C₆) alkyl, phenyl or benzyl ester or amide; or as an α-methylbenzyl amide). Other suitable amino and carboxy protecting groups are known to those skilled in the art (See for example, T. W. Greene, Protecting Groups In Organic Synthesis; Wiley: New York, 1981, and references cited therein). An amino acid can be linked to the remainder of a conjugate of formula I through the carboxy terminus, the amino terminus, or through any other convenient point of attachment, such as, for example, through the sulfur of a cysteine.

The terms “protein,” “peptide” and “polypeptide” are used interchangeably herein. Polypeptide sequences specifically recited herein are written with the amino terminus on the left and the carboxy terminus on the right.

The invention encompasses isolated or substantially purified nucleic acid or protein compositions. In the context of the present invention, an “isolated” or “purified” DNA molecule or an “isolated” or “purified” polypeptide is a DNA molecule or polypeptide that exists apart from its native environment and is therefore not a product of nature. An isolated DNA molecule or polypeptide may exist in a purified form or may exist in a non-native environment such as, for example, a transgenic host cell. For example, an “isolated” or “purified” nucleic acid molecule or protein, or biologically active portion thereof, is substantially free of other cellular material, or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. A protein that is substantially free of cellular material includes preparations of protein or polypeptide having less than about 30%, 20%, 10%, 5%, (by dry weight) of contaminating protein. When the protein of the invention, or biologically active portion thereof, is recombinantly produced, preferably culture medium represents less than about 30%, 20%, 10%, or 5% (by dry weight) of chemical precursors or non-protein-of-interest chemicals. Fragments and variants of the disclosed nucleotide sequences and proteins or partial-length proteins encoded thereby are also encompassed by the present invention. By “fragment” or “portion” is meant a full length or less than full length of the nucleotide sequence encoding, or the amino acid sequence of, a polypeptide or protein.

“Naturally occurring” is used to describe an object that can be found in nature as distinct from being artificially produced. For example, a protein or nucleotide sequence present in an organism (including a virus), which can be isolated from a source in nature and which has not been intentionally modified by man in the laboratory, is naturally occurring.

“Wild-type” refers to the normal gene, or organism found in nature without any known mutation.

A “variant” of a molecule is a sequence that is substantially similar to the sequence of the native molecule. For nucleotide sequences, variants include those sequences that, because of the degeneracy of the genetic code, encode the identical amino acid sequence of the native protein. Naturally occurring allelic variants such as these can be identified with the use of well-known molecular biology techniques, as, for example, with polymerase chain reaction (PCR) and hybridization techniques. Variant nucleotide sequences also include synthetically derived nucleotide sequences, such as those generated, for example, by using site-directed mutagenesis that encode the native protein, as well as those that encode a polypeptide having amino acid substitutions. Generally, nucleotide sequence variants of the invention will have at least 40, 50, 60, to 70%, e.g., preferably 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, to 98%, sequence identity to the native (endogenous) nucleotide sequence.

“Conservatively modified variations” of a particular nucleic acid sequence refers to those nucleic acid sequences that encode identical or essentially identical amino acid sequences, or where the nucleic acid sequence does not encode an amino acid sequence, to essentially identical sequences. Because of the degeneracy of the genetic code, a large number of functionally identical nucleic acids encode any given polypeptide. For instance, the codons CGT, CGC, CGA, CGG, AGA, and AGG all encode the amino acid arginine. Thus, at every position where an arginine is specified by a codon, the codon can be altered to any of the corresponding codons described without altering the encoded protein. Such nucleic acid variations are “silent variations” which are one species of “conservatively modified variations.” Every nucleic acid sequence described herein which encodes a polypeptide also describes every possible silent variation, except where otherwise noted. One of skill will recognize that each codon in a nucleic acid (except ATG, which is ordinarily the only codon for methionine) can be modified to yield a functionally identical molecule by standard techniques. Accordingly, each “silent variation” of a nucleic acid which encodes a polypeptide is implicit in each described sequence.

“Recombinant DNA molecule” is a combination of DNA sequences that are joined together using recombinant DNA technology and procedures used to join together DNA sequences as described, for example, in Sambrook and Russell, Molecular Cloning: A Laboratory Manual, Cold Spring Harbor, N.Y.: Cold Spring Harbor Laboratory Press (3^{rd} edition, 2001).

The terms “heterologous DNA sequence,” “exogenous DNA segment” or “heterologous nucleic acid,” each refer to a sequence that originates from a source foreign to the particular host cell or, if from the same source, is modified from its original form. Thus, a heterologous gene in a host cell includes a gene that is endogenous to the particular host cell but has been modified. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides.

A “homologous” DNA sequence is a DNA sequence that is naturally associated with a host cell into which it is introduced.

The term “gene” is used broadly to refer to any segment of nucleic acid associated with a biological function. Genes include coding sequences and/or the regulatory sequences required for their expression. For example, gene refers to a nucleic acid fragment that expresses mRNA, functional RNA, or a specific protein, including its regulatory sequences. Genes also include nonexpressed DNA segments that, for example, form recognition sequences for other proteins. Genes can be obtained from a variety of sources, including cloning from a source of interest or synthesizing from known or predicted sequence information, and may include sequences designed to have desired parameters. In addition, a “gene” or a “recombinant gene” refers to a nucleic acid molecule comprising an open reading frame and including at least one exon and (optionally) an intron sequence. The term “intron” refers to a DNA sequence present in a given gene which is not translated into protein and is generally found between exons.

A “vector” is defined to include, inter alia, any viral vector, plasmid, cosmid, phage or binary vector in double or single stranded linear or circular form which may or may not be self-transmissible or mobilizable, and which can transform prokaryotic or eukaryotic host either by integration into the cellular genome or exist extrachromosomally (e.g., autonomous replicating plasmid with an origin of replication).

“Cloning vectors” typically contain one or a small number of restriction endonuclease recognition sites at which foreign DNA sequences can be inserted in a determinable fashion without loss of essential biological function of the vector, as well as a marker gene that is suitable for use in the identification and selection of cells transformed with the cloning vector. Marker genes typically include genes that provide tetracycline resistance, hygromycin resistance or ampicillin resistance.

“Expression cassette” as used herein means a DNA sequence capable of directing expression of a particular nucleotide sequence in an appropriate host cell, comprising a promoter operably linked to the nucleotide sequence of interest which is operably linked to termination signals. It also typically comprises sequences required for proper translation of the nucleotide sequence. The coding region usually codes for a protein of interest but may also code for a functional RNA of interest, for example antisense RNA or a nontranslated RNA, in the sense or antisense direction. The expression cassette comprising the nucleotide sequence of interest may be chimeric, meaning that at least one of its components is heterologous with respect to at least one of its other components. The expression cassette may also be one that is naturally occurring but has been obtained in a recombinant form useful for heterologous expression. The expression of the nucleotide sequence in the expression cassette may be under the control of a constitutive promoter or of an inducible promoter that initiates transcription only when the host cell is exposed to some particular external stimulus. In the case of a multicellular organism, the promoter can also be specific to a particular tissue or organ or stage of development.

Such expression cassettes will comprise the transcriptional initiation region of the invention linked to a nucleotide sequence of interest. Such an expression cassette is provided with a plurality of restriction sites for insertion of the gene of interest to be under the transcriptional regulation of the regulatory regions. The expression cassette may additionally contain selectable marker genes.

The term “RNA transcript” refers to the product resulting from RNA polymerase catalyzed transcription of a DNA sequence. When the RNA transcript is a perfect complementary copy of the DNA sequence, it is referred to as the primary transcript or it may be a RNA sequence derived from posttranscriptional processing of the primary transcript and is referred to as the mature RNA. “Messenger RNA” (mRNA) refers to the RNA that is without introns and that can be translated into protein by the cell. “cDNA” refers to a single- or a double-stranded DNA that is complementary to and derived from mRNA.

“Regulatory sequences” and “suitable regulatory sequences” each refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences include enhancers, promoters, translation leader sequences, introns, and polyadenylation signal sequences. They include natural and synthetic sequences as well as sequences that may be a combination of synthetic and natural sequences. As is noted above, the term “suitable regulatory sequences” is not limited to promoters. However, some suitable regulatory sequences useful in the present invention will include, but are not limited to constitutive promoters, tissue-specific promoters, development-specific promoters, inducible promoters and viral promoters.

“5′ non-coding sequence” refers to a nucleotide sequence located 5′ (upstream) to the coding sequence. It is present in the fully processed mRNA upstream of the initiation codon and may affect processing of the primary transcript to mRNA, mRNA stability or translation efficiency (Turner et al. (1995) Mol. Biotech. 3:225).

“3′ non-coding sequence” refers to nucleotide sequences located 3′ (downstream) to a coding sequence and include polyadenylation signal sequences and other sequences encoding regulatory signals capable of affecting mRNA processing or gene expression. The polyadenylation signal is usually characterized by affecting the addition of polyadenylic acid tracts to the 3′ end of the mRNA precursor.

The term “translation leader sequence” refers to that DNA sequence portion of a gene between the promoter and coding sequence that is transcribed into RNA and is present in the fully processed mRNA upstream (5′) of the translation start codon. The translation leader sequence may affect processing of the primary transcript to mRNA, mRNA stability or translation efficiency.

The term “mature” protein refers to a post-translationally processed polypeptide without its signal peptide. “Precursor” protein refers to the primary product of translation of an mRNA. “Signal peptide” refers to the amino terminal extension of a polypeptide, which is translated in conjunction with the polypeptide forming a precursor peptide and which is required for its entrance into the secretory pathway. The term “signal sequence” refers to a nucleotide sequence that encodes the signal peptide.

“Promoter” refers to a nucleotide sequence, usually upstream (5′) to its coding sequence, which controls the expression of the coding sequence by providing the recognition for RNA polymerase and other factors required for proper transcription. “Promoter” includes a minimal promoter that is a short DNA sequence comprised of a TATA-box and other sequences that serve to specify the site of transcription initiation, to which regulatory elements are added for control of expression. “Promoter” also refers to a nucleotide sequence that includes a minimal promoter plus regulatory elements that is capable of controlling the expression of a coding sequence or functional RNA. This type of promoter sequence consists of proximal and more distal upstream elements, the latter elements often referred to as enhancers. Accordingly, an “enhancer” is a DNA sequence that can stimulate promoter activity and may be an innate element of the promoter or a heterologous element inserted to enhance the level or tissue specificity of a promoter. Promoters may be derived in their entirety from a native gene, or be composed of different elements derived from different promoters found in nature, or even be comprised of synthetic DNA segments. A promoter may also contain DNA sequences that are involved in the binding of protein factors that control the effectiveness of transcription initiation in response to physiological or developmental conditions.

The “initiation site” is the position surrounding the first nucleotide that is part of the transcribed sequence, which is also defined as position +1. With respect to this site all other sequences of the gene and its controlling regions are numbered. Downstream sequences (i.e. further protein encoding sequences in the 3′ direction) are denominated positive, while upstream sequences (mostly of the controlling regions in the 5′ direction) are denominated negative.

Promoter elements, particularly a TATA element, that are inactive or that have greatly reduced promoter activity in the absence of upstream activation are referred to as “minimal or core promoters.” In the presence of a suitable transcription factor, the minimal promoter functions to permit transcription. A “minimal or core promoter” thus consists only of all basal elements needed for transcription initiation, e.g., a TATA box and/or an initiator.

“Constitutive expression” refers to expression using a constitutive or regulated promoter. “Conditional” and “regulated expression” refer to expression controlled by a regulated promoter.

As used herein, the term “operably linked” refers to a linkage of two elements in a functional relationship. For example, “operably linked” may refer to a linkage of polynucleotide (or polypeptide) elements in a functional relationship. A nucleic acid is “operably linked” when it is placed into a functional relationship with another nucleic acid sequence. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. “Operably-linked” also refers to the association two chemical moieties so that the function of one is affected by the other, e.g., an arrangement of elements wherein the components so described are configured so as to perform their usual function.

“Expression” refers to the transcription and/or translation in a cell of an endogenous gene, transgene, as well as the transcription and stable accumulation of sense (mRNA) or functional RNA. In the case of antisense constructs, expression may refer to the transcription of the antisense DNA only. Expression may also refer to the production of protein.

“Transcription stop fragment” refers to nucleotide sequences that contain one or more regulatory signals, such as polyadenylation signal sequences, capable of terminating transcription. Examples of transcription stop fragments are known to the art.

“Translation stop fragment” refers to nucleotide sequences that contain one or more regulatory signals, such as one or more termination codons in all three frames, capable of terminating translation. Insertion of a translation stop fragment adjacent to or near the initiation codon at the 5′ end of the coding sequence will result in no translation or improper translation. Excision of the translation stop fragment by site-specific recombination will leave a site-specific sequence in the coding sequence that does not interfere with proper translation using the initiation codon.

“Homology” refers to the percent identity between two polynucleotides or two polypeptide sequences. Two DNA or polypeptide sequences are “homologous” to each other when the sequences exhibit at least about 75% to 85% (including 75%, 76%, 77%, 78%, 79%, 80%, 81%, 82%, 83%, 84%, and 85%), at least about 90%, or at least about 95% to 99% (including 95%, 96%, 97%, 98%, 99%) contiguous sequence identity over a defined length of the sequences.

The following terms are used to describe the sequence relationships between two or more sequences (e.g., nucleic acids, polynucleotides or polypeptides): (a) “reference sequence,” (b) “comparison window,” (c) “sequence identity,” (d) “percentage of sequence identity,” and (e) “substantial identity.”

(a) As used herein, “reference sequence” is a defined sequence used as a basis for sequence comparison. A reference sequence may be a subset or the entirety of a specified sequence; for example, as a segment of a full length cDNA, gene sequence or peptide sequence, or the complete cDNA, gene sequence or peptide sequence.

(b) As used herein, “comparison window” makes reference to a contiguous and specified segment of a sequence, wherein the sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the sequence a gap penalty is typically introduced and is subtracted from the number of matches.

Methods of alignment of sequences for comparison are well known in the art. Thus, the determination of percent identity between any two sequences can be accomplished using a mathematical algorithm. Non-limiting examples of such mathematical algorithms are the algorithm of Myers and Miller (1988) CABIOS, 4:11; the local homology algorithm of Smith et al. (1981) Adv. Appl. Math. 2:482; the homology alignment algorithm of Needleman and Wunsch, (1970) JMB, 48:443; the search-for-similarity-method of Pearson and Lipman, (1988) Proc. Natl. Acad. Sci. USA, 85:2444; the algorithm of Karlin and Altschul, (1990) Proc. Natl. Acad. Sci. USA, 87:2264, modified as in Karlin and Altschul, (1993) Proc. Natl. Acad. Sci. USA, 90:5873.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wis., USA). Alignments using these programs can be performed using the default parameters. The CLUSTAL program is well described by Higgins et al. (1988) Gene 73:237; Higgins et al. (1989) CABIOS 5:151; Corpet et al. (1988) Nucl. Acids Res. 16:10881; Huang et al. (1992) CABIOS 8:155; and Pearson et al. (1994) Meth. Mol. Biol. 24:307. The ALIGN program is based on the algorithm of Myers and Miller, supra. The BLAST programs of Altschul et al. (1990) JMB, 215:403; Nucl. Acids Res., 25:3389 (1990), are based on the algorithm of Karlin and Altschul supra.

Software for performing BLAST analyses is publicly available through the National Center for Biotechnology Information (available on the world wide web at ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached.

In addition to calculating percent sequence identity, the BLAST algorithm also performs a statistical analysis of the similarity between two sequences. One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, more preferably less than about 0.01, and most preferably less than about 0.001.

To obtain gapped alignments for comparison purposes, Gapped BLAST (in BLAST 2.0) can be utilized as described in Altschul et al. (1997) Nucleic Acids Res. 25:3389. Alternatively, PSI-BLAST (in BLAST 2.0) can be used to perform an iterated search that detects distant relationships between molecules. See Altschul et al., supra. When utilizing BLAST, Gapped BLAST, PSI-BLAST, the default parameters of the respective programs (e.g., BLASTN for nucleotide sequences, BLASTX for proteins) can be used. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix. See the world wide web at ncbi.nlm.nih.gov. Alignment may also be performed manually by visual inspection.

For purposes of the present invention, comparison of sequences for determination of percent sequence identity to another sequence may be made using the Blast program (e.g., BlastN, version 1.4.7 or later) with its default parameters or any equivalent program. By “equivalent program” is intended any sequence comparison program that, for any two sequences in question, generates an alignment having identical nucleotide or amino acid residue matches and an identical percent sequence identity when compared to the corresponding alignment generated by the preferred program.

(c) As used herein, “sequence identity” or “identity” in the context of two nucleic acid or polypeptide sequences makes reference to a specified percentage of residues in the two sequences that are the same when aligned for maximum correspondence over a specified comparison window, as measured by sequence comparison algorithms or by visual inspection. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

(e)(i) The term “substantial identity” of sequences means that a polynucleotide comprises a sequence that has at least 70%, 71%, 72%, 73%, 74%, 75%, 76%, 77%, 78%, or 79%, at least 80%, 81%, 82%, 83%, 84%, 85%, 86%, 87%, 88%, or 89%, at least 90%, 91%, 92%, 93%, or 94%, and at least 95%, 96%, 97%, 98%, or 99% sequence identity, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 70%, at least 80%, 90%, at least 95%.

Another indication that nucleotide sequences are substantially identical is if two molecules hybridize to each other under stringent conditions (see below). Generally, stringent conditions are selected to be about 5° C. lower than the thermal melting point (Tₘ) for the specific sequence at a defined ionic strength and pH. However, stringent conditions encompass temperatures in the range of about 1° C. to about 20° C., depending upon the desired degree of stringency as otherwise qualified herein. Nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code. One indication that two nucleic acid sequences are substantially identical is when the polypeptide encoded by the first nucleic acid is immunologically cross reactive with the polypeptide encoded by the second nucleic acid.

(e)(ii) The term “substantial identity” in the context of a peptide indicates that a peptide comprises a sequence with at least 70%, 71%, 72%, 73%, 74%, 75%, 76%, 77%, 78%, or 79%, 80%, 81%, 82%, 83%, 84%, 85%, 86%, 87%, 88%, or 89%, at least 90%, 91%, 92%, 93%, or 94%, or 95%, 96%, 97%, 98% or 99%, sequence identity to the reference sequence over a specified comparison window. Optimal alignment is conducted using the homology alignment algorithm of Needleman and Wunsch, J. Mol. Biol. 48:443 (1970). An indication that two peptide sequences are substantially identical is that one peptide is immunologically reactive with antibodies raised against the second peptide. Thus, a peptide is substantially identical to a second peptide, for example, where the two peptides differ only by a conservative substitution.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

As noted above, another indication that two nucleic acid sequences are substantially identical is that the two molecules hybridize to each other under stringent conditions. The phrase “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

By “variant” polypeptide is intended a polypeptide derived from the native protein by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein. Such variants may results form, for example, genetic polymorphism or from human manipulation. Methods for such manipulations are generally known in the art.

Thus, the polypeptides of the invention may be altered in various ways including amino acid substitutions, deletions, truncations, and insertions. Methods for such manipulations are generally known in the art. For example, amino acid sequence variants of the polypeptides can be prepared by mutations in the DNA. Methods for mutagenesis and nucleotide sequence alterations are well known in the art. See, for example, Kunkel (1985) Proc. Natl. Acad. Sci. USA 82:488; Kunkel et al. (1987) Meth. Enzymol. 154:367; U.S. Pat. No. 4,873,192; Walker and Gaastra (1983) Techniques in Mol. Biol. (MacMillan Publishing Co., and the references cited therein. Guidance as to appropriate amino acid substitutions that do not affect biological activity of the protein of interest may be found in the model of Dayhoff et al., Atlas of Protein Sequence and Structure (Natl. Biomed. Res. Found. 1978). Conservative substitutions, such as exchanging one amino acid with another having similar properties, are preferred.

Thus, the genes and nucleotide sequences of the invention include both the naturally occurring sequences as well as mutant forms. Likewise, the polypeptides of the invention encompass naturally occurring proteins as well as variations and modified forms thereof. Such variants will continue to possess the desired activity. In certain embodiments, the deletions, insertions, and substitutions of the polypeptide sequence encompassed herein may not produce radical changes in the characteristics of the polypeptide. However, when it is difficult to predict the exact effect of the substitution, deletion, or insertion in advance of doing so, one skilled in the art will appreciate that the effect will be evaluated by routine screening assays.

Individual substitutions deletions or additions that alter, add or delete a single amino acid or a small percentage of amino acids (typically less than 5%, more typically less than 1%) in an encoded sequence are “conservatively modified variations,” where the alterations result in the substitution of an amino acid with a chemically similar amino acid. Conservative substitution tables providing functionally similar amino acids are well known in the art. The following five groups each contain amino acids that are conservative substitutions for one another: Aliphatic: Glycine (G), Alanine (A), Valine (V), Leucine (L), Isoleucine (I); Aromatic: Phenylalanine (F), Tyrosine (Y), Tryptophan (W); Sulfur-containing: Methionine (M), Cysteine (C); Basic: Arginine (R), Lysine (K), Histidine (H); Acidic: Aspartic acid (D), Glutamic acid (E), Asparagine (N), Glutamine (Q). In addition, individual substitutions, deletions or additions which alter, add or delete a single amino acid or a small percentage of amino acids in an encoded sequence are also “conservatively modified variations.”

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” “transduced” and “recombinant” refer to a host cell or organism into which a heterologous nucleic acid molecule has been introduced. The nucleic acid molecule can be stably integrated into the genome generally known in the art and are disclosed in Sambrook and Russell, supra. See also Innis et al., PCR Protocols, Academic Press (1995); and Gelfand, PCR Strategies, Academic Press (1995); and Innis and Gelfand, PCR Methods Manual, Academic Press (1999). Known methods of PCR include, but are not limited to, methods using paired primers, nested primers, single specific primers, degenerate primers, gene-specific primers, vector-specific primers, partially mismatched primers, and the like. For example, “transformed,” “transformant,” and “transgenic” cells have been through the transformation process and contain a foreign gene integrated into their chromosome. The term “untransformed” refers to normal cells that have not been through the transformation process.

“Genetically altered cells” denotes cells which have been modified by the introduction of recombinant or heterologous nucleic acids (e.g., one or more DNA constructs or their RNA counterparts) and further includes the progeny of such cells which retain part or all of such genetic modification.

As used herein, the term “derived” or “directed to” with respect to a nucleotide molecule means that the molecule has complementary sequence identity to a particular molecule of interest.

As used herein, a “subject” is an animal, e.g., a mammal, e.g., a human, monkey, dog, cat, horse, cow, pig, goat, rabbit, or mouse.

Certain exemplary embodiments are as follows.

### Embodiment 1

A method for detecting an anti-SARS-CoV-2 antibody in a sample, the method comprising 1) contacting the sample with a polypeptide comprising a SARS-CoV-2 spike ectodomain amino acid sequence comprising a D614G mutation, under conditions suitable for an anti-SARS-CoV-2 antibody to bind to the polypeptide; and 2) detecting the presence of an anti-SARS-CoV-2 antibody bound to the polypeptide.

### Embodiment 2

The method of embodiment 1, wherein the sample is from an animal.

### Embodiment 3

The method of embodiment 2, wherein the animal has been vaccinated against SARS-CoV-2.

### Embodiment 4

A method for detecting a molecule associated with an immune response to SARS-CoV-2 or to a SARS-CoV-2 vaccine in an animal (e.g., human), the method comprising 1) contacting a sample from the animal with a polypeptide comprising a SARS-CoV-2 spike ectodomain amino acid sequence comprising a D614G mutation, under conditions suitable for the molecule to bind to the polypeptide; and 2) detecting the presence of the molecule bound to the polypeptide.

### Embodiment 5

The method of embodiment 4, wherein the molecule is an anti-SARS-CoV-2 antibody.

### Embodiment 6

The method of any one of embodiments 2-5, wherein the sample is from an animal that has been vaccinated against SARS-CoV-2.

### Embodiment 7

The method of any one of embodiments 1-3 and 5-6, wherein anti-SARS-CoV-2 antibody titers are measured.

### Embodiment 8

A method of diagnosing an animal as having or having had a SARS-CoV2 infection, the method comprising 1) obtaining a biological sample from the animal; 2) contacting the sample with a polypeptide comprising a SARS-CoV-2 spike ectodomain amino acid sequence comprising a D614G mutation and detecting whether anti-SARS-CoV-2 antibodies are present in the sample; and 3) diagnosing the animal as having or having had a SARS-CoV2 infection when anti-SARS-CoV-2 antibodies are detected.

### Embodiment 9

The method of embodiment 8, further comprising administering an anti-SARS-CoV-2 therapeutic agent to the animal.

### Embodiment 10

The method of any one of embodiments 1-9, wherein the detection step comprises an immunoassay.

### Embodiment 11

The method of any one of embodiments 1-10, wherein the polypeptide is resistant to furin cleavage.

### Embodiment 12

The method of any one of embodiments 1-11, wherein the polypeptide further comprises R682A, R683G and R685G mutations.

### Embodiment 13

The method of any one of embodiments 1-12, wherein the polypeptide further comprises K986P and V987P mutations.

### Embodiment 14

The method of any one of embodiments 1-13, wherein the spike ectodomain amino acid sequence is between about 1,180 to about 1,230 amino acids in length.

### Embodiment 15

The method of any one of embodiments 1-13, wherein the spike ectodomain amino acid sequence is between about 1,194 to about 1,216 amino acids in length.

### Embodiment 16

The method of any one of embodiments 1-15, wherein the spike ectodomain amino acid sequence comprises an amino acid sequence having at least about 80%, 85%, 90%, or 95% sequence identity to SEQ ID NO:3, and wherein the spike ectodomain amino acid sequence is further linked to 1 to 20 (e.g., consecutive) amino acids provided in a sequence corresponding to SEQ ID NO:24.

### Embodiment 17

The method of any one of embodiments 1-15, wherein the spike ectodomain amino acid sequence comprises an amino acid sequence having at least about 80%, 85%, 90%, or 95% sequence identity to any one of SEQ ID NOS:4-7 (e.g., SEQ ID NO:4 or 7).

### Embodiment 18

The method of embodiment 17, wherein the spike ectodomain amino acid sequence comprises an amino acid sequence having at least about 80%, 85%, 90%, or 95% sequence identity to SEQ ID NO:4 (about 95% sequence identity to SEQ ID NO:4).

### Embodiment 19

The method of embodiment 17, wherein the spike ectodomain amino acid sequence comprises SEQ ID NO:4.

### Embodiment 20

The method of embodiment 17, wherein the spike ectodomain amino acid sequence consists of SEQ ID NO:4.

### Embodiment 21

The method of embodiment 17, wherein the spike ectodomain amino acid sequence comprises an amino acid sequence having at least about 80%, 85%, 90%, or 95% sequence identity to SEQ ID NO:7.

### Embodiment 22

The method of embodiment 17, wherein the spike ectodomain amino acid sequence comprises SEQ ID NO:7.

### Embodiment 23

The method of embodiment 17, wherein the spike ectodomain amino acid sequence consists of SEQ ID NO:7.

### Embodiment 24

The method of any one of embodiments 1-23, wherein the polypeptide further comprises a trimerization motif, and wherein the trimerization motif is operably linked to the spike ectodomain amino acid sequence.

### Embodiment 25

The method of embodiment 24, wherein the trimerization motif is a foldon trimer motif (e.g., SEQ ID NO:16).

### Embodiment 26

The method of embodiment 24, wherein the trimerization motif is a GCN4 motif (e.g., SEQ ID NO:17).

### Embodiment 27

The method of any one of embodiments 24-26, wherein the trimerization motif is directly linked to the spike ectodomain amino acid sequence.

### Embodiment 28

The method of any one of embodiments 24-26, wherein the trimerization motif is linked to the spike ectodomain amino acid sequence through a linker group.

### Embodiment 29

The method of any one of embodiments 1-28, wherein the polypeptide is further operably linked to a detectable marker.

### Embodiment 30

The method of embodiment 29, wherein the detectable marker is directly linked to the spike ectodomain amino acid sequence.

### Embodiment 31

The method of embodiment 29, wherein the detectable marker is linked to the spike domain amino acid sequence through a linker group.

### Embodiment 32

The method of embodiment 31, wherein the linker group comprises a trimerization motif amino acid sequence (e.g., SEQ ID NO: 18 or 19).

### Embodiment 33

The method of any one of embodiments 29-32, wherein the detectable marker is a peptide tag.

### Embodiment 34

The method of embodiment 33, wherein the peptide tag is an affinity tag (e.g., a poly(His) tag).

### Embodiment 35

The method of any one of embodiments 1-34, wherein the polypeptide comprises an amino acid sequence having at least about 80%, 85%, 90%, or 95% sequence identity to SEQ ID NO:10 or 12.

### Embodiment 36

The method of embodiment 35, wherein the polypeptide comprises an amino acid sequence having at least about 80%, 85%, 90%, or 95% sequence identity to SEQ ID NO:10.

### Embodiment 37

The method of embodiment 35, wherein the polypeptide comprises SEQ ID NO:10.

### Embodiment 38

The method of embodiment 35, wherein the polypeptide consists of SEQ ID NO:10.

### Embodiment 39

The method of embodiment 35, wherein the polypeptide comprises an amino acid sequence having at least about 80%, 85%, 90%, or 95% sequence identity to SEQ ID NO:12.

### Embodiment 40

The method of embodiment 35, wherein the polypeptide comprises SEQ ID NO:12.

### Embodiment 41

The method of embodiment 35, wherein the polypeptide consists of SEQ ID NO:12.

### Embodiment 42

The method of any one of embodiments 1-41, wherein the polypeptide further comprises a signal peptide (e.g., a wildtype SARS-CoV-2 signal peptide).

### Embodiment 43

The method of embodiment 42, wherein the signal peptide is linked to the spike ectodomain amino acid sequence directly or through a linker group.

### Embodiment 44

The method of any one of embodiments 1-43, wherein the polypeptide comprises an amino acid sequence having at least about 80%, 85%, 90%, or 95% sequence identity to SEQ ID NO:11 or 13.

### Embodiment 45

The method of embodiment 44, wherein the polypeptide comprises an amino acid sequence having at least about 80%, 85%, 90%, or 95% sequence identity to SEQ ID NO:11.

### Embodiment 46

The method of embodiment 44, wherein the polypeptide comprises SEQ ID NO:11.

### Embodiment 47

The method of embodiment 44, wherein the polypeptide consists of SEQ ID NO:11.

### Embodiment 48

The method of embodiment 44, wherein the polypeptide comprises an amino acid sequence having at least about 80%, 85%, 90%, or 95% sequence identity to SEQ ID NO:13.

### Embodiment 49

The method of embodiment 44, wherein the polypeptide comprises SEQ ID NO:13.

### Embodiment 50

The method of embodiment 44, wherein the polypeptide consists of SEQ ID NO:13.

### Embodiment 51

The method of any one of embodiments 24-50, wherein the polypeptide is trimerized.

### Embodiment 52

The method of any one of embodiments 1-51, wherein the polypeptide or trimerized polypeptide is immobilized on a substrate.

### Embodiment 53

The method of embodiment 52, wherein the substrate is a plate, chip, or particle.

### Embodiment 54

A polypeptide comprising a SARS-CoV-2 spike ectodomain amino acid sequence comprising R682A, R683G and R685G mutations.

### Embodiment 55

The polypeptide of embodiment 54, further comprising a D614G mutation.

### Embodiment 56

The polypeptide of any one of embodiments 54-55, further comprising K986P and V987P mutations.

### Embodiment 57

The polypeptide of any one of embodiments 54-56, wherein the spike ectodomain amino acid sequence is between about 1,180 to about 1,230 amino acids in length.

### Embodiment 58

The polypeptide of any one of embodiments 54-56, wherein the spike ectodomain amino acid sequence is between about 1,194 to about 1,216 amino acids in length.

### Embodiment 59

The polypeptide of any one of embodiments 54-58, wherein the spike ectodomain comprises an amino acid sequence having at least about 80%, 85%, 90%, or 95% sequence identity to SEQ ID NO:3, and wherein spike ectodomain is further linked to 1 to 20 (e.g., consecutive) amino acids provided in a sequence corresponding to SEQ ID NO:24.

### Embodiment 60

The polypeptide of any one of embodiments 54-59, wherein the spike ectodomain amino acid sequence comprises an amino acid sequence having at least about 80%, 85%, 90%, or 95% sequence identity to SEQ ID NO:5, 6 or 7.

### Embodiment 61

The polypeptide of embodiment 60, wherein the spike ectodomain amino acid sequence comprises an amino acid sequence having at least about 80%, 85%, 90%, or 95% sequence identity to SEQ ID NO:7.

### Embodiment 62

The polypeptide of embodiment 60, wherein the spike ectodomain amino acid sequence comprises SEQ ID NO:7.

### Embodiment 63

The polypeptide of embodiment 60, wherein the spike ectodomain amino acid sequence consists of SEQ ID NO:7.

### Embodiment 64

The polypeptide of any one of embodiments 54-63, wherein the polypeptide further comprises a trimerization motif, wherein the trimerization motif is operably linked to the spike ectodomain amino acid sequence.

### Embodiment 65

The polypeptide of embodiment 64, wherein the trimerization motif is a foldon trimer motif. (e.g., SEQ ID NO:16).

### Embodiment 66

The polypeptide of embodiment 64, wherein the trimerization motif is a GCN4 motif (e.g., SEQ ID NO:17).

### Embodiment 67

The polypeptide of any one of embodiments 64-66, wherein the trimerization motif is directly linked to the spike ectodomain amino acid sequence.

### Embodiment 68

The polypeptide of any one of embodiments 64-66, wherein the trimerization motif is linked to the spike ectodomain amino acid sequence through a linker group.

### Embodiment 69

The polypeptide of any one of embodiments 54-68, wherein the polypeptide is further operably linked to a detectable marker.

### Embodiment 70

The polypeptide of embodiment 69, wherein the detectable marker is directly linked to the spike ectodomain amino acid sequence.

### Embodiment 71

The polypeptide of embodiment 69, wherein the detectable marker is linked to the spike domain amino acid sequence through a linker group.

### Embodiment 72

The polypeptide of embodiment 71, wherein the linker group comprises a trimerization motif amino acid sequence (e.g., SEQ ID NO: 18 or 19).

### Embodiment 73

The polypeptide of any one of embodiments 69-72, wherein the detectable marker is a peptide tag.

### Embodiment 74

The polypeptide of embodiment 73, wherein the peptide tag is an affinity tag (e.g., a poly(His) tag).

### Embodiment 75

The polypeptide of any one of embodiments 54-74, wherein the polypeptide comprises an amino acid sequence having at least about 80%, 85%, 90%, or 95% sequence identity to SEQ ID NO:8, 10, 12, or 25.

### Embodiment 76

The polypeptide of embodiment 75, wherein the polypeptide comprises an amino acid sequence having at least about 80%, 85%, 90%, or 95% sequence identity to SEQ ID NO:10.

### Embodiment 77

The polypeptide of embodiment 75, wherein the polypeptide comprises SEQ ID NO:10.

### Embodiment 78

The polypeptide of embodiment 75, wherein the polypeptide consists of SEQ ID NO:10.

### Embodiment 79

The polypeptide of embodiment 75, wherein the polypeptide comprises an amino acid sequence having at least about 80%, 85%, 90%, or 95% sequence identity to SEQ ID NO:12.

### Embodiment 80

The polypeptide of embodiment 75, wherein the polypeptide comprises SEQ ID NO:12.

### Embodiment 81

The polypeptide of embodiment 75, wherein the polypeptide consists of SEQ ID NO:12.

### Embodiment 82

The polypeptide of any one of embodiments 54-81, wherein the polypeptide further comprises a signal peptide (e.g., a wildtype SARS-CoV-2 signal peptide).

### Embodiment 83

The polypeptide of embodiment 82, wherein the signal peptide is linked to the spike ectodomain amino acid sequence directly or through a linker group.

### Embodiment 84

The polypeptide of any one of embodiments 54-83, wherein the polypeptide comprises an amino acid sequence having at least about 80%, 85%, 90%, or 95% sequence identity to SEQ ID NO:9, 11, 13, or 26.

### Embodiment 85

The polypeptide of embodiment 84, wherein the polypeptide comprises an amino acid sequence having at least about 80%, 85%, 90%, or 95% sequence identity to SEQ ID NO:11.

### Embodiment 86

The polypeptide of embodiment 84, wherein the polypeptide comprises SEQ ID NO:11.

### Embodiment 87

The polypeptide of embodiment 84, wherein the polypeptide consists of SEQ ID NO:11.

### Embodiment 88

The polypeptide of embodiment 84, wherein the polypeptide comprises an amino acid sequence having at least about 80%, 85%, 90%, or 95% sequence identity to SEQ ID NO:13.

### Embodiment 89

The polypeptide of embodiment 84, wherein the polypeptide comprises SEQ ID NO:13.

### Embodiment 90

The polypeptide of embodiment 84, wherein the polypeptide consists of SEQ ID NO:13.

### Embodiment 91

The polypeptide of any one of embodiments 64-90, wherein the polypeptide is trimerized.

### Embodiment 92

A diagnostic composition comprising a polypeptide or trimerized polypeptide as described in any one of embodiments 54-91 and a substrate, wherein the polypeptide or trimerized polypeptide is immobilized on the substrate.

### Embodiment 93

A composition comprising a polypeptide as described in any one of embodiments 54-91 and a carrier.

### Embodiment 94

The composition of embodiment 93, which is a diagnostic composition.

### Embodiment 95

An isolated polynucleotide comprising a nucleotide sequence encoding the polypeptide of any one of embodiments 54-91.

### Embodiment 96

The polynucleotide of embodiment 95, which comprises a nucleic acid sequence having at least about 90% sequence identity to SEQ ID NO:22 or 23.

### Embodiment 97

An expression cassette comprising a promoter operably linked to the polynucleotide of embodiment 95 or 96.

### Embodiment 98

A vector comprising the polynucleotide of embodiment 95 or 96 or the expression cassette of embodiment 97.

### Embodiment 99

A cell comprising the polynucleotide of embodiment 95 or 96, the expression cassette of embodiment 97 or the vector of embodiment 98.

### Embodiment 100

The cell of embodiment 99, which is a mammalian cell.

### Embodiment 101

The cell of embodiment 99, which is a human embryonic kidney (HEK) 293 cell (e.g., a 293F cell).

### Embodiment 102

A method of making a cell as described in any one of embodiments 99-101, the method comprising transfecting or transducing the cell with the polynucleotide of embodiment 95 or 96, the expression cassette of embodiment 97 or the vector of embodiment 98.

### Embodiment 103

The method of embodiment 102, further comprising using a selectable marker to select a cell that comprises the polynucleotide of embodiment 95 or 96, the expression cassette of embodiment 97 or the vector of embodiment 98.

### Embodiment 104

A method of producing a polypeptide, the method comprising transfecting or transducing a cell with the polynucleotide of embodiment 95 or 96, the expression cassette of embodiment 97 or the vector of embodiment 98.

### Embodiment 105

The method of embodiment 104, wherein the cell is a mammalian cell.

### Embodiment 106

The method of embodiment 105, wherein the cell is a human embryonic kidney (HEK) 293 cell (e.g., a 293F cell).

### Embodiment 107

The method of any one of embodiments 104-106, further comprising culturing the cell under appropriate conditions for expression of the polypeptide.

### Embodiment 108

A method of producing a polypeptide, the method comprising culturing a cell as described in any one of embodiments 99-101 under conditions appropriate for polypeptide expression.

### Embodiment 109

The method of any one of embodiments 104-108, further comprising isolating the polypeptide from the cell, cellular components and/or growth media.

### Embodiment 110

The method of any one of embodiments 104-109, further comprising purifying the isolated polypeptide.

### Embodiment 111

The method of embodiment 110, wherein the polypeptide is purified using an affinity column.

### Embodiment 112

The method of embodiment 110 or 111, wherein the polypeptide is purified using gel filtration.

### Embodiment 113

The method of any one of embodiments 110-112, wherein the purified protein comprises less than about 10% contaminants.

### Embodiment 114

The method of any one of embodiments 104-113, wherein the polypeptide is capable of binding to ACE2.

### Embodiment 115

A polypeptide produced by a method as described in any one of embodiments 104-114.

### Embodiment 116

A method for detecting an anti-SARS-CoV-2 antibody in a sample, the method comprising 1) contacting the sample with a polypeptide as described in any one of embodiments 54-91 and 115, under conditions suitable for an anti-SARS-CoV-2 antibody to bind to the polypeptide; and 2) detecting the presence of an anti-SARS-CoV-2 antibody bound to the polypeptide.

### Embodiment 117

A method for detecting a molecule associated with an immune response to SARS-CoV-2 or to a SARS-CoV-2 vaccine in an animal (e.g., human), the method comprising 1) contacting a sample from the animal with a polypeptide as described in any one of embodiments 54-91 and 115, under conditions suitable for the molecule to bind to the polypeptide; and 2) detecting the presence of the molecule bound to the polypeptide.

### Embodiment 118

A method of diagnosing an animal as having or having had a SARS-CoV2 infection, the method comprising 1) obtaining a biological sample from the animal; 2) contacting the sample with a polypeptide as described in any one of embodiments 54-91 and 115 and detecting whether anti-SARS-CoV-2 antibodies are present in the sample; and 3) diagnosing the animal as having or having had a SARS-CoV2 infection when anti-SARS-CoV-2 antibodies are detected.

### Embodiment 119

A kit comprising an isolated or purified SARS-CoV-2 spike ectodomain polypeptide of any one of embodiments 54-91 and 115, packaging material, and instructions for using the polypeptide in a method as described in any one of embodiments 116-118.

The invention will now be illustrated by the following non-limiting Examples.

### Example 1. Generation of Stable Cell Lines Expressing SARS-CoV2 Spike Ectodomain Polypeptide

The SARS-CoV-2 Spike protein (S protein) ectodomain is directly involved in the binding of SARS-CoV-2 to the host receptor ACE2 and virus cellular entry. As described below, mammalian 293F cells were genetically modified using a lentivirus system to stably express a SARS-CoV-2 spike ectodomain polypeptide.

### Materials and Methods

HEK293T cells were purchased from ATCC and 293F cells were purchased from Thermo Fisher Scientific.

### Cloning

Constructs encoding SEQ ID NO:9, SEQ ID NO:11, SEQ ID NO:13 and SEQ ID NO:26 were prepared. The constructs were cloned into a lentivirus vector (spike ectodomain plasmid). The plasmid was transformed into DH5α competent cells. Single bacterial colonies were picked for plasmids extraction. The plasmids were confirmed by DNA sequencing, and the correct constructs were used for further experiments.

### Preparation of Lentivirus Particles

HEK293T cells (5*10⁵ cells per well in 6 wells plate) were incubated at 37° C., 5% CO₂ overnight. The transfection was performed the next day using Lipofectamine 3000. Briefly, the spike ectodomain plasmid (1.5 ug) was mixed with 750 ng psPAX2 packaging plasmid and 250 ng pMD2.G envelope plasmid to 100u1 serum-free OPTI-MEN in a polypropylene tube. 5 ul P3000 was added to the plasmid mixture while diluting 5 ul lipofectamine 3000 with another 100u1 serum-free OPTI-MEN in another polypropylene tube. The plasmid cocktail was pipetted directly into the liquid containing the lipofectamine and then mixed by swirling or gently flicking the tube. The mixture was incubated for 10-15 minutes at room temperature and then the transfection mixture was gently added dropwise to the cells (the HEK293T cells should be 50-80% confluent). The cells were incubated at 37° C., 5% CO₂ for 24-48 hours. Media from the cells was harvested and transferred to a polypropylene storage tube. The harvested media, which contains the lentiviral particles, was stored at 4° C. (Note: for handling the lentivirus, the BSL2+ environment and following safety procedures are required).

### Infecting 293F Cells and Selection

30 ml of 293F cells (1*10⁶ cells/ml) were placed into a 125 ml shaking flask and Hexadimethrine bromide (polybrene) was added into media with a final concentration of 8 ug/ml. 1 ml of the harvested lentiviral particle solution from the above step was added to the flask. The cells were incubated at 37° C., 8% CO₂ in a shaking incubator overnight. Fresh 293F expression media was added the next morning; puromycin (1 ug/ml) was included in the media for selection. After two days, cells which contain the spike ectodomain gene will survive, while cells that do not contain the spike ectodomain gene will not survive. Fresh puromycin-containing media was provided as needed every few days. After almost one week, all the cells were selected and had turned into the spike ectodomain expressing cells.

### Protein Purification

(1) 293F cells stably expressing SARS-CoV-2 Spike ectodomain were grown in 293 FreeStyle expression medium at 37° C. with 8% CO₂ and 125 rpm in the CO₂ shaker. The cells were seeded in a 2 L flask at 0.5×10⁶ cells/ml.
(2) 6 days later, the cell culture supernatant was harvested: the cell culture was subjected to centrifuge at 6,000×g for 20 min at 4° C. The cell culture supernatant was collected.
(3) The cell culture supernatant was concentrated using Tangential Flow Filtration. During the concentration process, the buffer was changed from cell culture medium to PBS (phosphate-buffered saline) (to remove chemicals in the cell culture medium that may interfere with the subsequent protein purification steps). After concentration, the final volume of the sample was ˜300 ml (this volume may vary depending on the estimated amount of protein being purified). The sample was subjected to centrifugation at 12,000×g for 20 min at 4° C. The sample in the supernatant was collected. The sample was filtered using a 0.22 μm filter.
(4) The sample was loaded onto Nickel column (NiNTA column from GE healthcare; see GE product manual on details of preparing the column for protein purification).
(5) The protein was eluted off the Nickel column using gradient imidazole buffer. Buffer A: 20 mM Tris pH 7.2+500 mM NaCl. Buffer B: 20 mM Tris pH 7.2+500 mM NaCl+500 mM imidazole. The protein was eluted at 150-180 mM imidazole concentration.
(6) To check for the purity of the sample, the sample was run on SDS-PAGE and it was stained using Coomassie Blue. If the sample was pure enough (only one band on SDS-PAGE), then the method proceeded to step 8; however, if there were multiple bands on SDS-PAGE, then the method proceeded to go to step 7.
(7) The protein was optionally concentrated using Ultrafilter (Amicon) and gel filtration chromatography was run. Buffer was 20 mM Tris pH 7.2+200 mM NaCl.
(8) The protein was harvested and concentrated to ˜20 mg/ml using Ultrafilter (Amicon). If step 7 was skipped, during the concentration process, the buffer was changed to 20 mM Tris pH 7.2+200 mM NaCl.
(9) The protein's UV absorbance was measured (at 280 nm). The protein concentration was calculated based on the absorbance and its extinction coefficient (1 mg/ml protein has a UV absorbance of 1.00)
(10) The protein was flash frozen in liquid nitrogen and stored it at ˜80° C.

### Buffer Formulations

PBS (phosphate buffered saline, pH = 7.4):

| Salt | MW | Concentration (g/L) | Concentration (mol/L) |
|---|---|---|---|
| NaCl | 58.44 | 8.00669 | 0.1370 |
| Na₂HPO₄ | 141.95 | 1.41960 | 0.0100 |
| KCl | 74.55 | 0.20129 | 0.0027 |
| KH₂PO₄ | 136.08 | 0.24496 | 0.0018 |

Buffer A:

| Salt | MW | Concentration (g/L) | Concentration (mol/L) |
|---|---|---|---|
| NaCl | 58.44 | 29.22 | 0.500 |
| Tris-Base | 121.14 | 2.422 | 0.020 |
| | | | Adjust pH to 7.2 |

Buffer B:

| Salt | MW | Concentration (g/L) | Concentration (mol/L) |
|---|---|---|---|
| NaCl | 58.44 | 29.22 | 0.500 |
| Tris-Base | 121.14 | 2.422 | 0.020 |
| Imidazole | 68.08 | 34.04 | 0.500 |
| | | | Adjust pH to 7.2 |

Buffer A2 (gel-filtration buffer):

| Salt | MW | Concentration (g/L) | Concentration (mol/L) |
|---|---|---|---|
| NaCl | 58.44 | 11.69 | 0.200 |
| Tris-Base | 121.14 | 2.422 | 0.020 |
| | | | Adjust pH to 7.2 |

### Protein Purity and Quality Procedures (SDS-PAGE Method)

### SDS-PAGE Method:

1. 2 ug and bug purified protein sample was obtained and 4×loading buffer was added.
2. The sample was boiled for 5 min.
3. The samples were loaded into a SDS-PAGE gel, and the electrophoresis was started under 120V for 1 hour.
4. The gel was taken out and stained by Stain buffer (Coomassie blue) for 1 hour on a shaker with low speed.
5. The stain buffer was removed and washed a time by distill water.
6. De-stain buffer (30% methanol, 10% Acetic acid, 60% distill water) was added and placed on a low speed shaker for another 1 hour.
7. Step 6 was repeated until the gel become clear.
8. The purity was checked on a white plate.

### SDS-PAGE Materials:

SDS-PAGE gel, Electrophoresis system, Purified sample and
Sample loading buffer (Bid-Rad)
Stain buffer: 30% Methanol,
   10% Acetic Acid,
   60% distill water
   1.25% (w/v) Coomassie blue R250
Destain buffer: 30% Methanol,
   10% Acetic Acid,
   60% distill water

Results

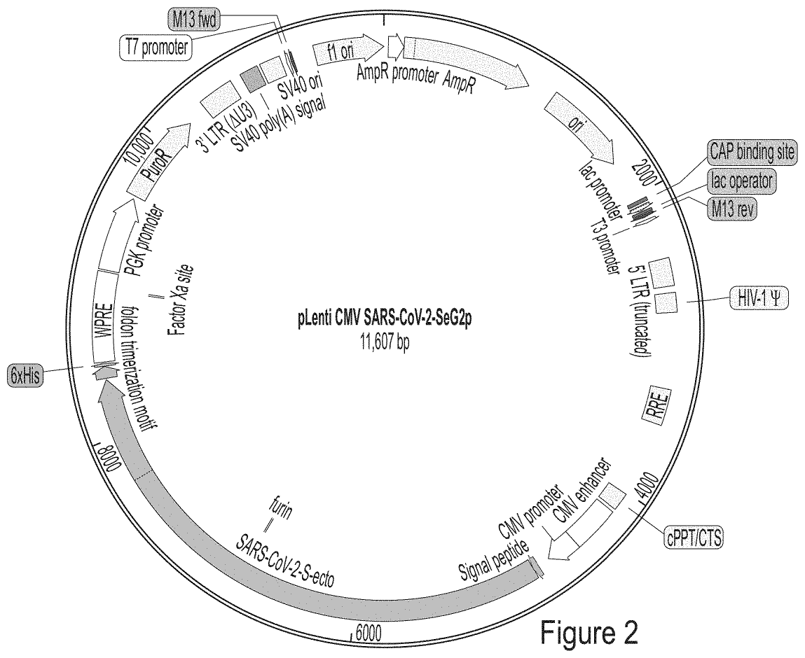

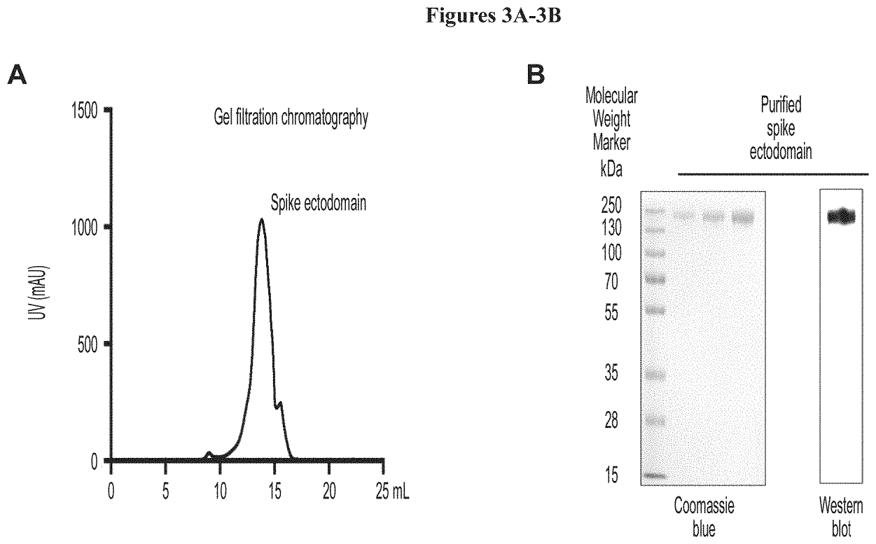

Mammalian 293F cells were genetically modified using a lentivirus system (e.g., FIG. 2) to stably express engineered SARS-CoV-2 spike ectodomain polypeptides (SEQ ID NO:9, 11, 13, or 26). It is noted that SEQ ID NOs: 9, 11, 13 and 26 refer to a polypeptide comprising a signal peptide; this signal peptide may be cleaved from the polypeptide prior to secretion. Accordingly, the purified polypeptide may be lacking the signal peptide (see, SEQ ID NO: 8, 10, 12, or 25). In particular, the expressed recombinant polypeptides in this Example comprised a SARS-CoV-2 spike ectodomain (SEQ ID NO:6 or 7) operably linked to a foldon trimer motif and a C-terminal 6× His tag (SEQ ID NO: 20), or comprised a SARS-CoV-2 spike ectodomain (SEQ ID NO:6 or 7) operably linked to a GCN4 motif and a C-terminal 6× His tag (SEQ ID NO: 20). The protein was purified sequentially using both a Nickel column and a gel filtration column. The homogeneity of the purified protein was confirmed by the elution profile of the gel filtration chromatography (FIG. 3A) and SDS-PAGE analysis (FIG. 3B).

Specifically, the first engineered control spike ectodomain polypeptide (SEQ ID NOs: 8 and 9) contains three mutations in the furin motif (R682A, R683G, R685G) and another two mutations in S2 (K986P, V987P).

Another engineered spike ectodomain polypeptide (SEQ ID NOs:10 and 11) contains a D614G mutation in addition to those five mutations in the control spike ectodomain polypeptide (SEQ ID NOs:8 and 9).

Surprisingly, the engineered spike ectodomain polypeptide construct that contains the D614G mutation showed a dramatic increase in protein yield (more than 20-fold increase in yield) compared to the control spike ectodomain polypeptide construct.

The protein yield from the engineered spike ectodomain polypeptide construct was ˜20 mg/liter cell culture, a vast improvement over the control spike ectodomain (typical yield was <1 mg/liter cell culture).

Compared to transiently transfected mammalian cells, the SARS-CoV-2 spike ectodomain polypeptide expressed from stably transfected mammalian cells can be quickly obtained, has a high yield and high purity, and is correctly folded and biologically active. Additionally, the SARS-CoV-2 spike ectodomain expressed from stably transfected mammalian cells has correctly added glycans (e.g., is better suited for assays) as compared to protein generated in other expression systems, such as insect cells. This system also allows production of SARS-CoV-2 spike ectodomain to be scaled up to suit industrial and commercial uses while maintaining the high quality of the product.

### Example 2. Enzyme-Linked Immunosorbent Assay (ELISA) Using Engineered SARS-CoV-2 Spike Ectodomain or RBD-Fc as ELISA Antigen

Mice were administered SARS-CoV-2 engineered control spike ectodomain (comprising SEQ ID NO:8), or RBD-Fc (10 μg antigen/mouse; 4 mice in each group).

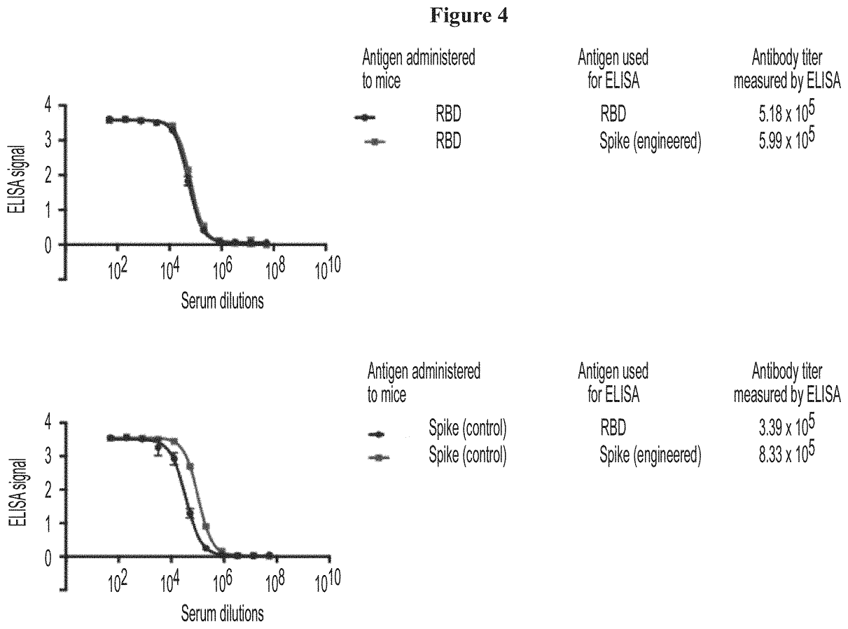

ELISA was performed (FIG. 4) for detecting the titers of RBD-targeting or spike-targeting IgG antibodies in the mouse sera. Two different antigens were used in the assay and coated on ELISA plates. One was the RBD (1 μg/ml or 40 μM for each monomeric RBD). The other was the engineered spike ectodomain (comprising SEQ ID NO:10) (5.33 μg/ml or 40 μM for each monomeric spike). Both antigens contain a C-terminal His6 tag (SEQ ID NO: 20). Serially diluted sera from each mouse group were added for detection of antigen/IgG binding. The titers were expressed as the endpoint dilutions that remain positively detectable. A titer was determined for sera from each group (sera from mice within each group were pooled together for this assay). Data are mean+S.E.M. All experiments were repeated independently three times with similar results.

The results (FIG. 4) showed that (i) for RBD-induced sera, the measured antibody titers using either RBD or engineered spike ectodomain (comprising SEQ ID NO:10) as the diagnostic antigen were similar. This is because only RBD-targeting antibodies were present and equal molar amounts of antigens were used in ELISA); (ii) for spike-induced sera, the measured antibody titer using the engineered spike ectodomain (comprising SEQ ID NO:10) as the diagnostic antigen was significantly higher than that using the RBD as the antigen. This is because antibodies targeting non-RBD parts of the spike ectodomain were present and were measured by the spike ectodomain as the antigen. In fact, RBD-targeting antibodies were less than half of the total spike-targeting antibodies. Therefore, compared to the RBD, the spike ectodomain is a more sensitive and specific antigen in diagnostic assays on virus-induced or spike-induced sera (as in the case of virus infections or vaccinations by Pfizer/Moderna spike mRNA vaccines, respectively).

### Analytical Procedures

### ELISA Method

1. SARS-CoV-2 RBD or S-ectodomain was diluted in coating buffer. The final concentration of each protein was 40 μM (S-ectodomain protein was calculated as monomer).
2. To coat protein on ELISA plate, 50 μl protein was added to each well and incubated at 4° C. overnight.
3. The coating buffer was removed and the ELISA plate was washed with PBS once.
4. Each well was blocked by adding 200 μl 3% BSA (or 5% non-fat milk, dilute in PBS).
5. The wells were washed with wash solution 3 times. The wash solution was removed.
6. Serum at different dilutions was added and then incubated for 1 hour at 37° C.
7. The wells were washed with wash solution 3 times and the wash solution removed.
8. An HRP conjugated anti-Fc antibody (1:1000 dilution in PBS) was added and incubated for 1 hour at 37° C.
9. The wells were washed with wash solution 4 times, and then the buffer was removed completely.
10. 50 μl ELISA substrate TMB was added to each well and incubated for 10 min.
11. The reaction was stopped by adding 50 μl H₂SO₄ (1 N) to each well.
12. The ELISA signal was detected using a plate reader (Tecan, San Jose, Calif.) at 450 nm.

### ELISA Materials

ELISA plate (half-area plate, Corning), BSA or non-fat milk, H2504 (1 N), PBS, Anti-mouse Fc or Anti-human Fc antibody (HRP conjugated, Jackson ImmunoResearch Lab, PA), ELISA substrate: 3,3′,5,5′-Tetramethylbenzidine (TMB) (Sigma, St. Louis, Mo.),

Coating buffer:

| Salt | MW | Concentration (g/L) | Concentration (mol/L) |
|---|---|---|---|
| Na₂CO₃ | 105.989 | 1.5 | 0.200 |
| NaHCO₃ | 84.007 | 2.93 | 0.020 |
| | | | Adjust pH to 9.6 |

### Wash Solution:

1 L of PBS+0.5 ml of Tween 20

### Example 3. Comparison of RBD and Spike Ectodomain as Antigens in COVID-19 Diagnostic Assays

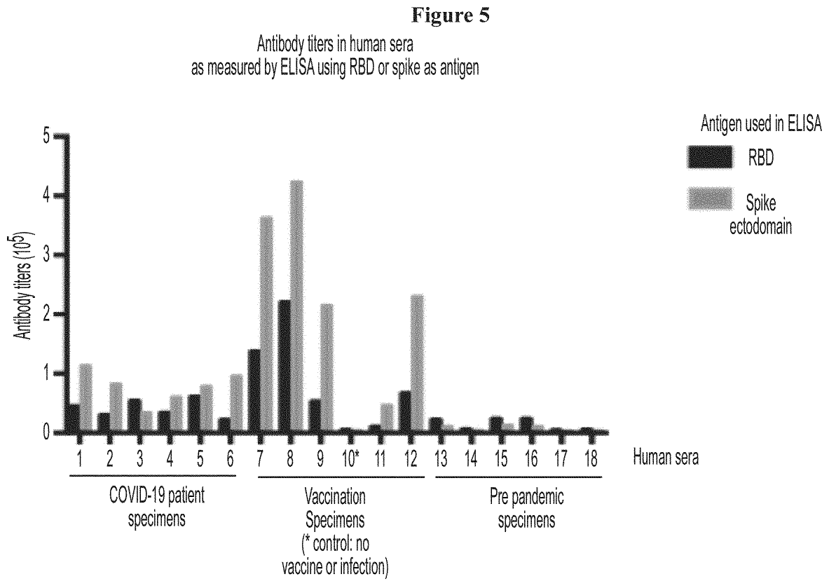

ELISA was performed (e.g., see FIG. 5) to detect the SARS-CoV-2-specific antibody titers in COVID-19 patients (samples #1-6), vaccinated people (samples #7-12, with #10 as a control), and pre-pandemic samples (see, ELISA method of Example 2). Two antigens, RBD and engineered spike ectodomain (comprising SEQ ID NO:10), were used for head-to-head comparisons (equal molar amounts of the antigens were coated).

The results showed that for samples from COVID-19 patients or vaccinated people, in which SARS-CoV-2-specific antibodies were present, the spike ectodomain gave significantly higher ELISA signals than the RBD. Conversely, for pre-pandemic samples in which SARS-CoV-2-specific antibodies were absent, the spike ectodomain gave significantly lower ELISA signals than the RBD. Therefore, due to its higher sensitivity and specificity, the spike ectodomain has significant advantages over RBD as a COVID-19 diagnostic antigen.

TABLE 1

| SEQ | | |
|---|---|---|
| ID NO: | Sequences | Comment |
| 1 | MFVFLVLLPLVSSQCVNLTTRTQLPPAYTNSFTRGVYYPDKVFRSSVL | SARS-CoV-2 Spike |
| | HSTQDLFLPFFSNVTWFHAIHVSGTNGTKRFDNPVLPFNDGVYFASTE | protein (wildtype) |
| | KSNIIRGWIFGTTLDSKTQSLLIVNNATNVVIKVCEFQFCNDPFLGVY | full-length amino acid |
| | YHKNNKSWMESEFRVYSSANNCTFEYVSQPFLMDLEGKQGNFKNLREF | sequence (1-1273). |
| | VFKNIDGYFKIYSKHTPINLVRDLPQGFSALEPLVDLPIGINITRFQT | The putative furin |
| | LLALHRSYLTPGDSSSGWTAGAAAYYVGYLQPRTFLLKYNENGTITDA | cleavage site [RRAR |
| | VDCALDPLSETKCTLKSFTVEKGIYQTSNFRVQPTESIVRFPNITNLC | (SEQ ID NO: 44)] is |
| | PFGEVFNATRFASVYAWNRKRISNCVADYSVLYNSASFSTFKCYGVSP | bolded. |
| | TKLNDLCFTNVYADSFVIRGDEVRQIAPGQTGKIADYNYKLPDDFTGC | The signal peptide is |
| | VIAWNSNNLDSKVGGNYNYLYRLFRKSNLKPFERDISTEIYQAGSTPC | bolded and underlined |
| | NGVEGFNCYFPLQSYGFQPTNGVGYQPYRVVVLSFELLHAPATVCGPK | |
| | KSTNLVKNKCVNFNFNGLTGTGVLTESNKKFLPFQQFGRDIADTTDAV | |
| | RDPQTLEILDITPCSFGGVSVITPGTNTSNQVAVLYQDVNCTEVPVAI | |
| | HADQLTPTWRVYSTGSNVFQTRAGCLIGAEHVNNSYECDIPIGAGICA | |
| | SYQTQTNSPRRARSVASQSIIAYTMSLGAENSVAYSNNSIAIPTNFTI | |
| | SVTTEILPVSMTKTSVDCTMYICGDSTECSNLLLQYGSFCTQLNRALT | |
| | GIAVEQDKNTQEVFAQVKQIYKTPPIKDFGGFNFSQILPDPSKPSKRS | |
| | FIEDLLFNKVTLADAGFIKQYGDCLGDIAARDLICAQKFNGLTVLPPL | |
| | LTDEMIAQYTSALLAGTITSGWTFGAGAALQIPFAMQMAYRFNGIGVT | |
| | QNVLYENQKLIANQFNSAIGKIQDSLSSTASALGKLQDVVNQNAQALN | |
| | TLVKQLSSNFGAISSVLNDILSRLDKVEAEVQIDRLITGRLQSLQTYV | |
| | TQQLIRAAEIRASANLAATKMSECVLGQSKRVDFCGKGYHLMSFPQSA | |
| | PHGVVFLHVTYVPAQEKNFTTAPAICHDGKAHFPREGVFVSNGTHWFV | |
| | TQRNFYEPQIITTDNTFVSGNCDVVIGIVNNTVYDPLQPELDSFKEEL | |
| | DKYFKNHTSPDVDLGDISGINASVVNIQKEIDRLNEVAKNLNESLIDL | |
| | QELGKYEQYIKWPWYIWLGFIAGLIAIVMVTIMLCCMTSCCSCLKGCC | |
| | SCGSCCKFDEDDSEPVLKGVKLHYT | |
| 2 | MFVFLVLLPLVSSQCVNLTTRTQLPPAYTNSFTRGVYYPDKVFRSSVL | SARS-CoV-2 spike |
| | HSTQDLFLPFFSNVTWFHAIHVSGTNGTKRFDNPVLPFNDGVYFASTE | ectodomain (wildtype) |
| | KSNIIRGWIFGTTLDSKTQSLLIVNNATNVVIKVCEFQFCNDPFLGVY | amino acid sequence |
| | YHKNNKSWMESEFRVYSSANNCTFEYVSQPFLMDLEGKQGNFKNLREF | (1-1211). |
| | VFKNIDGYFKIYSKHTPINLVRDLPQGFSALEPLVDLPIGINITRFQT | The putative furin |
| | LLALHRSYLTPGDSSSGWTAGAAAYYVGYLQPRTFLLKYNENGTITDA | cleavage sie [RRAR |
| | VDCALDPLSETKCTLKSFTVEKGIYQTSNFRVQPTESIVRFPNITNLC | (SEQ ID NO: 44)] is |
| | PFGEVFNATRFASVYAWNRKRISNCVADYSVLYNSASFSTFKCYGVSP | bolded. |
| | TKLNDLCFTNVYADSFVIRGDEVRQIAPGQTGKIADYNYKLPDDFTGC | The signal peptide is |
| | VIAWNSNNLDSKVGGNYNYLYRLFRKSNLKPFERDISTEIYQAGSTPC | bolded and underlined. |
| | NGVEGFNCYFPLQSYGFQPTNGVGYQPYRVVVLSFELLHAPATVCGPK | |
| | KSTNLVKNKCVNFNFNGLTGTGVLTESNKKFLPFQQFGRDIADTTDAV | |
| | RDPQTLEILDITPCSFGGVSVITPGTNTSNQVAVLYQDVNCTEVPVAI | |
| | HADQLTPTWRVYSTGSNVFQTRAGCLIGAEHVNNSYECDIPIGAGICA | |
| | SYQTQTNSPRRARSVASQSIIAYTMSLGAENSVAYSNNSIAIPTNFTI | |
| | SVTTEILPVSMTKTSVDCTMYICGDSTECSNLLLQYGSFCTQLNRALT | |
| | GIAVEQDKNTQEVFAQVKQIYKTPPIKDFGGFNFSQILPDPSKPSKRS | |
| | FIEDLLFNKVTLADAGFIKQYGDCLGDIAARDLICAQKFNGLTVLPPL | |
| | LTDEMIAQYTSALLAGTITSGWTFGAGAALQIPFAMQMAYRFNGIGVT | |
| | QNVLYENQKLIANQFNSAIGKIQDSLSSTASALGKLQDVVNQNAQALN | |
| | TLVKQLSSNFGAISSVLNDILSRLDKVEAEVQIDRLITGRLQSLQTYV | |
| | TQQLIRAAEIRASANLAATKMSECVLGQSKRVDFCGKGYHLMSFPQSA | |
| | PHGVVFLHVTYVPAQEKNFTTAPAICHDGKAHFPREGVFVSNGTHWFV | |
| | TQRNFYEPQIITTDNTFVSGNCDVVIGIVNNTVYDPLQPELDSFKEEL | |
| | DKYFKNHTSPDVDLGDISGINASVVNIQKEIDRLNEVAKNLNESLIDL | |
| | QELGKYEQYIK | |
| 3 | QCVNLTTRTQLPPAYTNSFTRGVYYPDKVFRSSVLHSTQDLFLPFFSN | One embodiment of a |
| | VTWFHAIHVSGTNGTKRFDNPVLPFNDGVYFASTEKSNIIRGWIFGTT | SARS-CoV-2 spike |
| | LDSKTQSLLIVNNATNVVIKVCEFQFCNDPFLGVYYHKNNKSWMESEF | ectodomain (wildtype) |
| | RVYSSANNCTFEYVSQPFLMDLEGKQGNFKNLREFVFKNIDGYFKIYS | amino acid sequence |
| | KHTPINLVRDLPQGFSALEPLVDLPIGINITRFQTLLALHRSYLTPGD | (residues 14-1201). |
| | SSSGWTAGAAAYYVGYLQPRTFLLKYNENGTITDAVDCALDPLSETKC | |
| | TLKSFTVEKGIYQTSNFRVQPTESIVRFPNITNLCPFGEVFNATRFAS | |
| | VYAWNRKRISNCVADYSVLYNSASFSTFKCYGVSPTKLNDLCFTNVYA | |
| | DSFVIRGDEVRQIAPGQTGKIADYNYKLPDDFTGCVIAWNSNNLDSKV | |
| | GGNYNYLYRLFRKSNLKPFERDISTEIYQAGSTPCNGVEGFNCYFPLQ | |
| | SYGFQPTNGVGYQPYRVVVLSFELLHAPATVCGPKKSTNLVKNKCVNF | |
| | NFNGLTGTGVLTESNKKFLPFQQFGRDIADTTDAVRDPQTLEILDITP | |
| | CSFGGVSVITPGTNTSNQVAVLYQDVNCTEVPVAIHADQLTPTWRVYS | |
| | TGSNVFQTRAGCLIGAEHVNNSYECDIPIGAGICASYQTQTNSPRRAR | |
| | SVASQSIIAYTMSLGAENSVAYSNNSIAIPTNFTISVTTEILPVSMTK | |
| | TSVDCTMYICGDSTECSNLLLQYGSFCTQLNRALTGIAVEQDKNTQEV | |
| | FAQVKQIYKTPPIKDFGGFNFSQILPDPSKPSKRSFIEDLLFNKVTLA | |
| | DAGFIKQYGDCLGDIAARDLICAQKFNGLTVLPPLLTDEMIAQYTSAL | |
| | LAGTITSGWTFGAGAALQIPFAMQMAYRFNGIGVTQNVLYENQKLIAN | |
| | QFNSAIGKIQDSLSSTASALGKLQDVVNQNAQALNTLVKQLSSNFGAI | |
| | SSVLNDILSRLDKVEAEVQIDRLITGRLQSLQTYVTQQLIRAAEIRAS | |
| | ANLAATKMSECVLGQSKRVDFCGKGYHLMSFPQSAPHGVVFLHVTYVP | |
| | AQEKNFTTAPAICHDGKAHFPREGVFVSNGTHWFVTQRNFYEPQIITT | |
| | DNTFVSGNCDVVIGIVNNTVYDPLQPELDSFKEELDKYFKNHTSPDVD | |
| | LGDISGINASVVNIQKEIDRLNEVAKNLNESLIDLQ | |
| 4 | QCVNLTTRTQLPPAYTNSFTRGVYYPDKVFRSSVLHSTQDLFLPFFSN | One embodiment of |
| | VTWFHAIHVSGTNGTKRFDNPVLPFNDGVYFASTEKSNIIRGWIFGTT | engineered SARS-CoV-2 |
| | LDSKTQSLLIVNNATNVVIKVCEFQFCNDPFLGVYYHKNNKSWMESEF | spike ectodomain amino |
| | RVYSSANNCTFEYVSQPFLMDLEGKQGNFKNLREFVFKNIDGYFKIYS | acid sequence (14- |
| | KHTPINLVRDLPQGFSALEPLVDLPIGINITRFQTLLALHRSYLTPGD | 1211) comprising |
| | SSSGWTAGAAAYYVGYLQPRTFLLKYNENGTITDAVDCALDPLSETKC | D614G. |
| | TLKSFTVEKGIYQTSNFRVQPTESIVRFPNITNLCPFGEVFNATRFAS | |
| | VYAWNRKRISNCVADYSVLYNSASFSTFKCYGVSPTKLNDLCFTNVYA | Mutations are |
| | DSFVIRGDEVRQIAPGQTGKIADYNYKLPDDFTGCVIAWNSNNLDSKV | underlined. |
| | GGNYNYLYRLFRKSNLKPFERDISTEIYQAGSTPCNGVEGFNCYFPLQ | |
| | SYGFQPTNGVGYQPYRVVVLSFELLHAPATVCGPKKSTNLVKNKCVNF | |
| | NFNGLTGTGVLTESNKKFLPFQQFGRDIADTTDAVRDPQTLEILDITP | |
| | CSFGGVSVITPGTNTSNQVAVLYQGVNCTEVPVAIHADQLTPTWRVYS | |
| | TGSNVFQTRAGCLIGAEHVNNSYECDIPIGAGICASYQTQTNSPRRAR | |
| | SVASQSIIAYTMSLGAENSVAYSNNSIAIPTNFTISVTTEILPVSMTK | |
| | TSVDCTMYICGDSTECSNLLLQYGSFCTQLNRALTGIAVEQDKNTQEV | |
| | FAQVKQIYKTPPIKDFGGFNFSQILPDPSKPSKRSFIEDLLFNKVTLA | |
| | DAGFIKQYGDCLGDIAARDLICAQKFNGLTVLPPLLTDEMIAQYTSAL | |
| | LAGTITSGWTFGAGAALQIPFAMQMAYRFNGIGVTQNVLYENQKLIAN | |
| | QFNSAIGKIQDSLSSTASALGKLQDVVNQNAQALNTLVKQLSSNFGAI | |
| | SSVLNDILSRLDKVEAEVQIDRLITGRLQSLQTYVTQQLIRAAEIRAS | |
| | ANLAATKMSECVLGQSKRVDFCGKGYHLMSFPQSAPHGVVFLHVTYVP | |
| | AQEKNFTTAPAICHDGKAHFPREGVFVSNGTHWFVTQRNFYEPQIITT | |
| | DNTFVSGNCDVVIGIVNNTVYDPLQPELDSFKEELDKYFKNHTSPDVD | |
| | LGDISGINASVVNIQKEIDRLNEVAKNLNESLIDLQELGKYEQYIK | |
| 5 | VTWFHAIHVSGTNGTKRFDNPVLPFNDGVYFASTEKSNIIRGWIFGTT | One embodiment of |
| | LDSKTQSLLIVNNATNVVIKVCEFQFCNDPFLGVYYHKNNKSWMESEF | engineered SARS-CoV-2 |
| | RVYSSANNCTFEYVSQPFLMDLEGKQGNFKNLREFVFKNIDGYFKIYS | spike ectodomain amino |
| | KHTPINLVRDLPQGFSALEPLVDLPIGINITRFQTLLALHRSYLTPGD | acid sequence (14- |
| | SSSGWTAGAAAYYVGYLQPRTFLLKYNENGTITDAVDCALDPLSETKC | 1211) comprising: |
| | TLKSFTVEKGIYQTSNFRVQPTESIVRFPNITNLCPFGEVFNATRFAS | furin cleavage site |
| | VYAWNRKRISNCVADYSVLYNSASFSTFKCYGVSPTKLNDLCFTNVYA | mutations (R682A, |
| | DSFVIRGDEVRQIAPGQTGKIADYNYKLPDDFTGCVIAWNSNNLDSKV | R683G, R685G). |
| | GGNYNYLYRLFRKSNLKPFERDISTEIYQAGSTPCNGVEGFNCYFPLQ | Mutations are |
| | SYGFQPTNGVGYQPYRVVVLSFELLHAPATVCGPKKSTNLVKNKCVNF | underlined. |
| | NFNGLTGTGVLTESNKKFLPFQQFGRDIADTTDAVRDPQTLEILDITP | |
| | CSFGGVSVITPGTNTSNQVAVLYQDVNCTEVPVAIHADQLTPTWRVYS | |
| | TGSNVFQTRAGCLIGAEHVNNSYECDIPIGAGICASYQTQTNSPAGAG | |
| | SVASQSIIAYTMSLGAENSVAYSNNSIAIPTNFTISVTTEILPVSMTK | |
| | TSVDCTMYICGDSTECSNLLLQYGSFCTQLNRALTGIAVEQDKNTQEV | |
| | FAQVKQIYKTPPIKDFGGFNFSQILPDPSKPSKRSFIEDLLFNKVTLA | |
| | DAGFIKQYGDCLGDIAARDLICAQKFNGLTVLPPLLTDEMIAQYTSAL | |
| | LAGTITSGWTFGAGAALQIPFAMQMAYRFNGIGVTQNVLYENQKLIAN | |
| | QFNSAIGKIQDSLSSTASALGKLQDVVNQNAQALNTLVKQLSSNFGAI | |
| | SSVLNDILSRLDKVEAEVQIDRLITGRLQSLQTYVTQQLIRAAEIRAS | |
| | ANLAATKMSECVLGQSKRVDFCGKGYHLMSFPQSAPHGVVFLHVTYVP | |
| | AQEKNFTTAPAICHDGKAHFPREGVFVSNGTHWFVTQRNFYEPQIITT | |
| | DNTFVSGNCDVVIGIVNNTVYDPLQPELDSFKEELDKYFKNHTSPDVD | |
| | LGDISGINASVVNIQKEIDRLNEVAKNLNESLIDLQELGKYEQYIK | |
| 6 | VTWFHAIHVSGTNGTKRFDNPVLPFNDGVYFASTEKSNIIRGWIFGTT | One embodiment of |
| | LDSKTQSLLIVNNATNVVIKVCEFQFCNDPFLGVYYHKNNKSWMESEF | engineered SARS-CoV-2 |
| | RVYSSANNCTFEYVSQPFLMDLEGKQGNFKNLREFVFKNIDGYFKIYS | spike ectodomain amino |
| | KHTPINLVRDLPQGFSALEPLVDLPIGINITRFQTLLALHRSYLTPGD | acid sequence (14- |
| | SSSGWTAGAAAYYVGYLQPRTFLLKYNENGTITDAVDCALDPLSETKC | 1211) comprising: |
| | TLKSFTVEKGIYQTSNFRVQPTESIVRFPNITNLCPFGEVFNATRFAS | 1) furin cleavage site |
| | VYAWNRKRISNCVADYSVLYNSASFSTFKCYGVSPTKLNDLCFTNVYA | mutations (R682A, |
| | DSFVIRGDEVRQIAPGQTGKIADYNYKLPDDFTGCVIAWNSNNLDSKV | R683G, R685G), and |
| | GGNYNYLYRLFRKSNLKPFERDISTEIYQAGSTPCNGVEGFNCYFPLQ | 2) a stabilizing dual |
| | SYGFQPTNGVGYQPYRVVVLSFELLHAPATVCGPKKSTNLVKNKCVNF | mutation (K986P, |
| | NFNGLTGTGVLTESNKKFLPFQQFGRDIADTTDAVRDPQTLEILDITP | V987P). |
| | CSFGGVSVITPGTNTSNQVAVLYQDVNCTEVPVAIHADQLTPTWRVYS | Mutations are |
| | TGSNVFQTRAGCLIGAEHVNNSYECDIPIGAGICASYQTQTNSPAGAG | underlined. |
| | SVASQSIIAYTMSLGAENSVAYSNNSIAIPTNFTISVTTEILPVSMTK | |
| | TSVDCTMYICGDSTECSNLLLQYGSFCTQLNRALTGIAVEQDKNTQEV | |
| | FAQVKQIYKTPPIKDFGGFNFSQILPDPSKPSKRSFIEDLLFNKVTLA | |
| | DAGFIKQYGDCLGDIAARDLICAQKFNGLTVLPPLLTDEMIAQYTSAL | |
| | LAGTITSGWTFGAGAALQIPFAMQMAYRFNGIGVTQNVLYENQKLIAN | |
| | QFNSAIGKIQDSLSSTASALGKLQDVVNQNAQALNTLVKQLSSNFGAI | |
| | SSVLNDILSRLDPPEAEVQIDRLITGRLQSLQTYVTQQLIRAAEIRAS | |
| | ANLAATKMSECVLGQSKRVDFCGKGYHLMSFPQSAPHGVVFLHVTYVP | |
| | AQEKNFTTAPAICHDGKAHFPREGVFVSNGTHWFVTQRNFYEPQIITT | |
| | DNTFVSGNCDVVIGIVNNTVYDPLQPELDSFKEELDKYFKNHTSPDVD | |
| | LGDISGINASVVNIQKEIDRLNEVAKNLNESLIDLQELGKYEQYIK | |
| 7 | QCVNLTTRTQLPPAYTNSFTRGVYYPDKVFRSSVLHSTQDLFLPFFSN | One embodiment of |
| | VTWFHAIHVSGTNGTKRFDNPVLPFNDGVYFASTEKSNIIRGWIFGTT | engineered SARS-CoV-2 |
| | LDSKTQSLLIVNNATNVVIKVCEFQFCNDPFLGVYYHKNNKSWMESEF | spike ectodomain amino |
| | RVYSSANNCTFEYVSQPFLMDLEGKQGNFKNLREFVFKNIDGYFKIYS | acid sequence (14- |
| | KHTPINLVRDLPQGFSALEPLVDLPIGINITRFQTLLALHRSYLTPGD | 1211) comprising: |
| | SSSGWTAGAAAYYVGYLQPRTFLLKYNENGTITDAVDCALDPLSETKC | 1) D614G, |
| | TLKSFTVEKGIYQTSNFRVQPTESIVRFPNITNLCPFGEVFNATRFAS | 2) furin cleavage site |
| | VYAWNRKRISNCVADYSVLYNSASFSTFKCYGVSPTKLNDLCFTNVYA | mutations (R682A, |
| | DSFVIRGDEVRQIAPGQTGKIADYNYKLPDDFTGCVIAWNSNNLDSKV | R683G, R685G), and |
| | GGNYNYLYRLFRKSNLKPFERDISTEIYQAGSTPCNGVEGFNCYFPLQ | 3) stabilizing dual |
| | SYGFQPTNGVGYQPYRVVVLSFELLHAPATVCGPKKSTNLVKNKCVNF | mutation (K986P, |
| | NFNGLTGTGVLTESNKKFLPFQQFGRDIADTTDAVRDPQTLEILDITP | V987P). |
| | CSFGGVSVITPGTNTSNQVAVLYQGVNCTEVPVAIHADQLTPTWRVYS | Mutations are |
| | TGSNVFQTRAGCLIGAEHVNNSYEEDIPIGAGICASYQTQTNSPAGAG | underlined. |
| | SVASQSIIAYTMSLGAENSVAYSNNSIAIPTNFTISVTTEILPVSMTK | |
| | TSVDCTMYICGDSTECSNLLLQYGSFCTQLNRALTGIAVEQDKNTQEV | |
| | FAQVKQIYKTPPIKDFGGFNFSQILPDPSKPSKRSFIEDLLFNKVTLA | |
| | DAGFIKQYGDCLGDIAARDLICAQKFNGLTVLPPLLTDEMIAQYTSAL | |
| | LAGTITSGWTFGAGAALQIPFAMQMAYRFNGIGVTQNVLYENQKLIAN | |
| | QFNSAIGKIQDSLSSTASALGKLQDVVNQNAQALNTLVKQLSSNFGAI | |
| | SSVLNDILSRLDPPEAEVQIDRLITGRLQSLQTYVTQQLIRAAEIRAS | |
| | ANLAATKMSECVLGQSKRVDFCGKGYHLMSFPQSAPHGVVFLHVTYVP | |
| | AQEKNFTTAPAICHDGKAHFPREGVFVSNGTHWFVTQRNFYEPQIITT | |
| | DNTFVSGNCDVVIGIVNNTVYDPLQPELDSFKEELDKYFKNHTSPDVD | |
| | LGDISGINASVVNIQKEIDRLNEVAKNLNESLIDLQELGKYEQYIK | |
| 8 | QCVNLTTRTQLPPAYTNSFTRGVYYPDKVFRSSVLHSTQDLFLPFFSN | One embodiment of |
| | VTWFHAIHVSGTNGTKRFDNPVLPFNDGVYFASTEKSNIIRGWIFGTT | engineered SARS-CoV-2 |
| | LDSKTQSLLIVNNATNVVIKVCEFQFCNDPFLGVYYHKNNKSWMESEF | spike ectodomain (SEQ |
| | RVYSSANNCTFEYVSQPFLMDLEGKQGNFKNLREFVFKNIDGYFKIYS | ID NO: 6) operably |
| | KHTPINLVRDLPQGFSALEPLVDLPIGINITRFQTLLALHRSYLTPGD | linked to a foldon |
| | SSSGWTAGAAAYYVGYLQPRTFLLKYNENGTITDAVDCALDPLSETKC | trimer motif, |
| | TLKSFTVEKGIYQTSNFRVQPTESIVRFPNITNLCPFGEVFNATRFAS | comprising: |
| | VYAWNRKRISNCVADYSVLYNSASFSTFKCYGVSPTKLNDLCFTNVYA | 1) furin cleavage site |
| | DSFVIRGDEVRQIAPGQTGKIADYNYKLPDDFTGCVIAWNSNNLDSKV | mutations (R682A, |
| | GGNYNYLYRLFRKSNLKPFERDISTEIYQAGSTPCNGVEGFNCYFPLQ | R683G, R685G), and |
| | SYGFQPTNGVGYQPYRVVVLSFELLHAPATVCGPKKSTNLVKNKCVNF | 2) stabilizing dual |
| | NFNGLTGTGVLTESNKKFLPFQQFGRDIADTTDAVRDPQTLEILDITP | mutation (K986P, |
| | CSFGGVSVITPGTNTSNQVAVLYQDVNCTEVPVAIHADQLTPTWRVYS | V987P). |
| | TGSNVFQTRAGCLIGAEHVNNSYECDIPIGAGICASYQTQTNSPAGAG | Linker GSG (SEQ ID NO: |
| | SVASQSIIAYTMSLGAENSVAYSNNSIAIPTNFTISVTTEILPVSMTK | 15) is bolded. |
| | TSVDCTMYICGDSTECSNLLLQYGSFCTQLNRALTGIAVEQDKNTQEV | Foldon trimer motif is |
| | FAQVKQIYKTPPIKDFGGFNFSQILPDPSKPSKRSFIEDLLFNKVTLA | italicized. |
| | DAGFIKQYGDCLGDIAARDLICAQKFNGLTVLPPLLTDEMIAQYTSAL | |
| | LAGTITSGWTFGAGAALQIPFAMQMAYRFNGIGVTQNVLYENQKLIAN | |
| | QFNSAIGKIQDSLSSTASALGKLQDVVNQNAQALNTLVKQLSSNFGAI | |
| | SSVLNDILSRLDPPEAEVQIDRLITGRLQSLQTYVTQQLIRAAEIRAS | |
| | ANLAATKMSECVLGQSKRVDFCGKGYHLMSFPQSAPHGVVFLHVTYVP | |
| | AQEKNFTTAPAICHDGKAHFPREGVFVSNGTHWFVTQRNFYEPQIITT | |
| | DNTFVSGNCDVVIGIVNNTVYDPLQPELDSFKEELDKYFKNHTSPDVD | |
| | LGDISGINASVVNIQKEIDRLNEVAKNLNESLIDLQELGKYEQYIKGS | |
| | GYIPEAPRDGQAYVREDGEWVLLSTFLGHHHHHH | |
| 9 | MFVFLVLLPLVSSQCVNLTTRTQLPPAYTNSFTRGVYYPDKVFRSSVL | One embodiment of |
| | HSTQDLFLPFFSNVTWFHAIHVSGTNGTKRFDNPVLPFNDGVYFASTE | engineered SARS-CoV-2 |
| | YHKNNKSWMESEFRVYSSANNCTFEYVSQPFLMDLEGKQGNFKNLREF | spike ectodomain (SEQ |
| | VFKNIDGYFKIYSKHTPINLVRDLPQGFSALEPLVDLPIGINITRFQT | ID NO: 6 + signal |
| | LLALHRSYLTPGDSSSGWTAGAAAYYVGYLQPRTFLLKYNENGTITDA | peptide) operably |
| | VDCALDPLSETKCTLKSFTVEKGIYQTSNFRVQPTESIVRFPNITNLC | linked to a foldon |
| | PFGEVFNATRFASVYAWNRKRISNCVADYSVLYNSASFSTFKCYGVSP | trimer motif, |
| | TKLNDLCFTNVYADSFVIRGDEVRQIAPGQTGKIADYNYKLPDDFTGC | comprising: |
| | VIAWNSNNLDSKVGGNYNYLYRLFRKSNLKPFERDISTEIYQAGSTPC | 1) furin cleavage site |
| | NGVEGFNCYFPLQSYGFQPTNGVGYQPYRVVVLSFELLHAPATVCGPK | mutations (R682A, |
| | KSTNLVKNKCVNFNFNGLTGTGVLTESNKKFLPFQQFGRDIADTTDAV | R683G, R685G), and |
| | RDPQTLEILDITPCSFGGVSVITPGTNTSNQVAVLYQDVNCTEVPVAI | 2) stabilizing dual |
| | HADQLTPTWRVYSTGSNVFQTRAGCLIGAEHVNNSYECDIPIGAGICA | mutation (K986P, |
| | SYQTQTNSPAGAGSVASQSIIAYTMSLGAENSVAYSNNSIAIPTNFTI | V987P). |
| | SVTTEILPVSMTKTSVDCTMYICGDSTECSNLLLQYGSFCTQLNRALT | Linker GSG (SEQ ID NO: |
| | GIAVEQDKNTQEVFAQVKQIYKTPPIKDFGGFNFSQILPDPSKPSKRS | 15) is bolded. |
| | FIEDLLFNKVTLADAGFIKQYGDCLGDIAARDLICAQKFNGLTVLPPL | Signal peptide is |
| | LTDEMIAQYTSALLAGTITSGWTFGAGAALQIPFAMQMAYRFNGIGVT | bolded and underlined |
| | QNVLYENQKLIANQFNSAIGKIQDSLSSTASALGKLQDVVNQNAQALN | Foldon trimer motif is |
| | TLVKQLSSNFGAISSVLNDILSRLDPPEAEVQIDRLITGRLQSLQTYV | italicized. |
| | TQQLIRAAEIRASANLAATKMSECVLGQSKRVDFCGKGYHLMSFPQSA | |
| | PHGVVFLHVTYVPAQEKNFTTAPAICHDGKAHFPREGVFVSNGTHWFV | |
| | TQRNFYEPQIITTDNTFVSGNCDVVIGIVNNTVYDPLQPELDSFKEEL | |
| | DKYFKNHTSPDVDLGDISGINASVVNIQKEIDRLNEVAKNLNESLIDL | |
| | QELGKYEQYIKGSGYIPEAPRDGQAYVREDGEWVLLSTFLGHHHHHH | |
| 10 | QCVNLTTRTQLPPAYTNSFTRGVYYPDKVFRSSVLHSTQDLFLPFFSN | One embodiment of |
| | VTWFHAIHVSGTNGTKRFDNPVLPFNDGVYFASTEKSNIIRGWIFGTT | engineered SARS-CoV-2 |
| | LDSKTQSLLIVNNATNVVIKVCEFQFCNDPFLGVYYHKNNKSWMESEF | spike ectodomain (SEQ |
| | RVYSSANNCTFEYVSQPFLMDLEGKQGNFKNLREFVFKNIDGYFKIYS | ID NO: 7) operably |
| | KHTPINLVRDLPQGFSALEPLVDLPIGINITRFQTLLALHRSYLTPGD | linked to a foldon |
| | SSSGWTAGAAAYYVGYLQPRTFLLKYNENGTITDAVDCALDPLSETKC | trimer motif, |
| | TLKSFTVEKGIYQTSNFRVQPTESIVRFPNITNLCPFGEVFNATRFAS | comprising: |
| | VYAWNRKRISNCVADYSVLYNSASFSTFKCYGVSPTKLNDLCFTNVYA | 1) D614G, |
| | DSFVIRGDEVRQIAPGQTGKIADYNYKLPDDFTGCVIAWNSNNLDSKV | 2) furin cleavage site |
| | GGNYNYLYRLFRKSNLKPFERDISTEIYQAGSTPCNGVEGFNCYFPLQ | mutations (R682A, |
| | SYGFQPTNGVGYQPYRVVVLSFELLHAPATVCGPKKSTNLVKNKCVNF | R683G, R685G), and |
| | NFNGLTGTGVLTESNKKFLPFQQFGRDIADTTDAVRDPQTLEILDITP | 3) stabilizing dual |
| | CSFGGVSVITPGTNTSNQVAVLYQGVNCTEVPVAIHADQLTPTWRVYS | mutation (K986P, |
| | TGSNVFQTRAGCLIGAEHVNNSYEEDIPIGAGICASYQTQTNSPAGAG | V987P). |
| | SVASQSIIAYTMSLGAENSVAYSNNSIAIPTNFTISVTTEILPVSMTK | Linker GSG (SEQ ID NO: |
| | TSVDCTMYICGDSTECSNLLLQYGSFCTQLNRALTGIAVEQDKNTQEV | 15) is bolded. |
| | FAQVKQIYKTPPIKDFGGFNFSQILPDPSKPSKRSFIEDLLFNKVTLA | Foldon trimer motif is |
| | DAGFIKQYGDCLGDIAARDLICAQKFNGLTVLPPLLTDEMIAQYTSAL | italicized. |
| | LAGTITSGWTFGAGAALQIPFAMQMAYRFNGIGVTQNVLYENQKLIAN | |
| | QFNSAIGKIQDSLSSTASALGKLQDVVNQNAQALNTLVKQLSSNFGAI | |
| | SSVLNDILSRLDPPEAEVQIDRLITGRLQSLQTYVTQQLIRAAEIRAS | |
| | ANLAATKMSECVLGQSKRVDFCGKGYHLMSFPQSAPHGVVFLHVTYVP | |
| | AQEKNFTTAPAICHDGKAHFPREGVFVSNGTHWFVTQRNFYEPQIITT | |
| | DNTFVSGNCDVVIGIVNNTVYDPLQPELDSFKEELDKYFKNHTSPDVD | |
| | LGDISGINASVVNIQKEIDRLNEVAKNLNESLIDLQELGKYEQYIKGS | |
| | GYIPEAPRDGQAYVREDGEWVLLSTFLGHHHHHH | |
| 11 | MFVFLVLLPLVSSQCVNLTTRTQLPPAYTNSFTRGVYYPDKVFRSSVL | One embodiment of |
| | HSTQDLFLPFFSNVTWFHAIHVSGTNGTKRFDNPVLPFNDGVYFASTE | engineered SARS-CoV-2 |
| | KSNIIRGWIFGTTLDSKTQSLLIVNNATNVVIKVCEFQFCNDPFLGVY | spike ectodomain (SEQ |
| | YHKNNKSWMESEFRVYSSANNCTFEYVSQPFLMDLEGKQGNFKNLREF | ID NO: 7 + signal |
| | VFKNIDGYFKIYSKHTPINLVRDLPQGFSALEPLVDLPIGINITRFQT | peptide) operably |
| | LLALHRSYLTPGDSSSGWTAGAAAYYVGYLQPRTFLLKYNENGTITDA | linked to a foldon |
| | VDCALDPLSETKCTLKSFTVEKGIYQTSNFRVQPTESIVRFPNITNLC | trimer motif, |
| | PFGEVFNATRFASVYAWNRKRISNCVADYSVLYNSASFSTFKCYGVSP | comprising: |
| | TKLNDLCFTNVYADSFVIRGDEVRQIAPGQTGKIADYNYKLPDDFTGC | 1) D614G, |
| | VIAWNSNNLDSKVGGNYNYLYRLFRKSNLKPFERDISTEIYQAGSTPC | 2) furin cleavage site |
| | NGVEGFNCYFPLQSYGFQPTNGVGYQPYRVVVLSFELLHAPATVCGPK | mutations (R682A, |
| | KSTNLVKNKCVNFNFNGLTGTGVLTESNKKFLPFQQFGRDIADTTDAV | R683G, R685G), and |
| | RDPQTLEILDITPCSFGGVSVITPGTNTSNQVAVLYQGVNCTEVPVAI | 3) stabilizing dual |
| | HADQLTPTWRVYSTGSNVFQTRAGCLIGAEHVNNSYECDIPIGAGICA | mutation (K986P, |
| | SYQTQTNSPAGAGSVASQSIIAYTMSLGAENSVAYSNNSIAIPTNFTI | V987P). |
| | SVTTEILPVSMTKTSVDCTMYICGDSTECSNLLLQYGSFCTQLNRALT | Linker GSG (SEQ ID NO: |
| | GIAVEQDKNTQEVFAQVKQIYKTPPIKDFGGFNFSQILPDPSKPSKRS | 15) is bolded. |
| | FIEDLLFNKVTLADAGFIKQYGDCLGDIAARDLICAQKFNGLTVLPPL | Signal peptide is |
| | LTDEMIAQYTSALLAGTITSGWTFGAGAALQIPFAMQMAYRFNGIGVT | bolded and underlined. |
| | QNVLYENQKLIANQFNSAIGKIQDSLSSTASALGKLQDVVNQNAQALN | Foldon trimer motif is |
| | TLVKQLSSNFGAISSVLNDILSRLDPPEAEVQIDRLITGRLQSLQTYV | italicized. |
| | TQQLIRAAEIRASANLAATKMSECVLGQSKRVDFCGKGYHLMSFPQSA | |
| | PHGVVFLHVTYVPAQEKNFTTAPAICHDGKAHFPREGVFVSNGTHWFV | |
| | TQRNFYEPQIITTDNTFVSGNCDVVIGIVNNTVYDPLQPELDSFKEEL | |
| | DKYFKNHTSPDVDLGDISGINASVVNIQKEIDRLNEVAKNLNESLIDL | |
| | QELGKYEQYIKGSGYIPEAPRDGQAYVREDGEWVLLSTFLGHHHHHH | |
| 12 | QCVNLTTRTQLPPAYTNSFTRGVYYPDKVFRSSVLHSTQDLFLPFFSN | One embodiment of |
| | VTWFHAIHVSGTNGTKRFDNPVLPFNDGVYFASTEKSNIIRGWIFGTT | engineered SARS-CoV-2 |
| | LDSKTQSLLIVNNATNVVIKVCEFQFCNDPFLGVYYHKNNKSWMESEF | spike ectodomain (SEQ |
| | RVYSSANNCTFEYVSQPFLMDLEGKQGNFKNLREFVFKNIDGYFKIYS | ID NO: 7) operably |
| | KHTPINLVRDLPQGFSALEPLVDLPIGINITRFQTLLALHRSYLTPGD | linked to a GCN4 |
| | SSSGWTAGAAAYYVGYLQPRTFLLKYNENGTITDAVDCALDPLSETKC | trimer motif, |
| | TLKSFTVEKGIYQTSNFRVQPTESIVRFPNITNLCPFGEVFNATRFAS | comprising: |
| | VYAWNRKRISNCVADYSVLYNSASFSTFKCYGVSPTKLNDLCFTNVYA | 1) D614G, |
| | DSFVIRGDEVRQIAPGQTGKIADYNYKLPDDFTGCVIAWNSNNLDSKV | 2) furin cleavage site |
| | GGNYNYLYRLFRKSNLKPFERDISTEIYQAGSTPCNGVEGFNCYFPLQ | mutations (R682A, |
| | SYGFQPTNGVGYQPYRVVVLSFELLHAPATVCGPKKSTNLVKNKCVNF | R683G, R685G), and |
| | NFNGLTGTGVLTESNKKFLPFQQFGRDIADTTDAVRDPQTLEILDITP | 3) stabilizing dual |
| | CSFGGVSVITPGTNTSNQVAVLYQGVNCTEVPVAIHADQLTPTWRVYS | mutation (K986P, |
| | TGSNVFQTRAGCLIGAEHVNNSYECDIPIGAGICASYQTQTNSPAGAG | V987P). |
| | SVASQSIIAYTMSLGAENSVAYSNNSIAIPTNFTISVTTEILPVSMTK | Linker GSG (SEQ ID NO: |
| | TSVDCTMYICGDSTECSNLLLQYGSFCTQLNRALTGIAVEQDKNTQEV | 15) is bolded. |
| | FAQVKQIYKTPPIKDFGGFNFSQILPDPSKPSKRSFIEDLLFNKVTLA | GCN4 trimer motif is |
| | DAGFIKQYGDCLGDIAARDLICAQKFNGLTVLPPLLTDEMIAQYTSAL | italicized, |
| | LAGTITSGWTFGAGAALQIPFAMQMAYRFNGIGVTQNVLYENQKLIAN | |
| | QFNSAIGKIQDSLSSTASALGKLQDVVNQNAQALNTLVKQLSSNFGAI | |
| | SSVLNDILSRLDPPEAEVQIDRLITGRLQSLQTYVTQQLIRAAEIRAS | |
| | ANLAATKMSECVLGQSKRVDFCGKGYHLMSFPQSAPHGVVFLHVTYVP | |
| | AQEKNFTTAPAICHDGKAHFPREGVFVSNGTHWFVTQRNFYEPQIITT | |
| | DNTFVSGNCDVVIGIVNNTVYDPLQPELDSFKEELDKYFKNHTSPDVD | |
| | LGDISGINASVVNIQKEIDRLNEVAKNLNESLIDLQELGKYEQYIKGS | |
| | GIKRMKQIEDKIEEIESKQKKIENEIARIKKIKGHHHHHH | |
| 13 | MFVFLVLLPLVSSQCVNLTTRTQLPPAYTNSFTRGVYYPDKVFRSSVL | One embodiment of |
| | HSTQDLFLPFFSNVTWFHAIHVSGTNGTKRFDNPVLPFNDGVYFASTE | engineered SARS-CoV-2 |
| | KSNIIRGWIFGTTLDSKTQSLLIVNNATNVVIKVCEFQFCNDPFLGVY | spike ectodomain (SEQ |
| | YHKNNKSWMESEFRVYSSANNCTFEYVSQPFLMDLEGKQGNFKNLREF | ID NO: 7) + Signal |
| | VFKNIDGYFKIYSKHTPINLVRDLPQGFSALEPLVDLPIGINITRFQT | peptide) operably |
| | LLALHRSYLTPGDSSSGWTAGAAAYYVGYLQPRTFLLKYNENGTITDA | linked to a GCN4 |
| | VDCALDPLSETKCTLKSFTVEKGIYQTSNFRVQPTESIVRFPNITNLC | trimer motif, |
| | PFGEVFNATRFASVYAWNRKRISNCVADYSVLYNSASFSTFKCYGVSP | comprising: |
| | TKLNDLCFTNVYADSFVIRGDEVRQIAPGQTGKIADYNYKLPDDFTGC | 1) D614G, |
| | VIAWNSNNLDSKVGGNYNYLYRLFRKSNLKPFERDISTEIYQAGSTPC | 2) furin cleavage site |
| | NGVEGFNCYFPLQSYGFQPTNGVGYQPYRVVVLSFELLHAPATVCGPK | mutations (R682A, |
| | KSTNLVKNKCVNFNFNGLTGTGVLTESNKKFLPFQQFGRDIADTTDAV | R683G, R685G), and |
| | RDPQTLEILDITPCSFGGVSVITPGTNTSNQVAVLYQGVNCTEVPVAI | 3) stabilizing dual |
| | HADQLTPTWRVYSTGSNVFQTRAGCLIGAEHVNNSYEEDIPIGAGICA | mutation (K986P, |
| | SYQTQTNSPAGAGSVASQSIIAYTMSLGAENSVAYSNNSIAIPTNFTI | V987P). |
| | SVTTEILPVSMTKTSVDCTMYICGDSTECSNLLLQYGSFCTQLNRALT | Linker GSG (SEQ ID NO: |
| | GIAVEQDKNTQEVFAQVKQIYKTPPIKDFGGFNFSQILPDPSKPSKRS | 15) is bolded. |
| | FIEDLLFNKVTLADAGFIKQYGDCLGDIAARDLICAQKFNGLTVLPPL | GCN4 trimer motif is |
| | LTDEMIAQYTSALLAGTITSGWTFGAGAALQIPFAMQMAYRFNGIGVT | italicized. |
| | QNVLYENQKLIANQFNSAIGKIQDSLSSTASALGKLQDVVNQNAQALN | |
| | TLVKQLSSNFGAISSVLNDILSRLDPPEAEVQIDRLITGRLQSLQTYV | |
| | TQQLIRAAEIRASANLAATKMSECVLGQSKRVDFCGKGYHLMSFPQSA | |
| | PHGVVELHVTYVPAQEKNETTAPAICHDGKAHFPREGVEVSNGTHWEV | |
| | TQRNFYEPQIITTDNTFVSGNCDVVIGIVNNTVYDPLQPELDSFKEEL | |
| | DKYFKNHTSPDVDLGDISGINASVVNIQKEIDRLNEVAKNLNESLIDL | |
| | QELGKYEQYIKGSGIKRMKQIEDKIEEIESKQKKIENEIARIKKIKGH | |
| | HHHHH | |
| 25 | QCVNLTTRTQLPPAYTNSFTRGVYYPDKVFRSSVLHSTQDLFLPFFSN | One embodiment of |
| | VTWFHAIHVSGTNGTKRFDNPVLPFNDGVYFASTEKSNIIRGWIFGTT | engineered SARS-CoV-2 |
| | LDSKTQSLLIVNNATNVVIKVCEFQFCNDPFLGVYYHKNNKSWMESEF | spike ectodomain (SEQ |
| | RVYSSANNCTFEYVSQPFLMDLEGKQGNFKNLREFVFKNIDGYFKIYS | ID NO: 6) operably |
| | KHTPINLVRDLPQGFSALEPLVDLPIGINITRFQTLLALHRSYLTPGD | linked to a GCN4 |
| | SSSGWTAGAAAYYVGYLQPRTFLLKYNENGTITDAVDCALDPLSETKC | trimer motif, |
| | TLKSFTVEKGIYQTSNFRVQPTESIVRFPNITNLCPFGEVFNATRFAS | comprising: |
| | VYAWNRKRISNCVADYSVLYNSASFSTFKCYGVSPTKLNDLCFTNVYA | 1) furin cleavage site |
| | DSFVIRGDEVRQIAPGQTGKIADYNYKLPDDFTGCVIAWNSNNLDSKV | mutations (R682A, |
| | GGNYNYLYRLFRKSNLKPFERDISTEIYQAGSTPCNGVEGFNCYFPLQ | R683G, R685G), and |
| | SYGFQPTNGVGYQPYRVVVLSFELLHAPATVCGPKKSTNLVKNKCVNF | 2) stabilizing dual |
| | NFNGLTGTGVLTESNKKFLPFQQFGRDIADTTDAVRDPQTLEILDITP | mutation (K986P, |
| | CSFGGVSVITPGTNTSNQVAVLYQDVNCTEVPVAIHADQLTPTWRVYS | V987P). |
| | TGSNVFQTRAGCLIGAEHVNNSYECDIPIGAGICASYQTQTNSPAGAG | Linker GSG (SEQ ID NO: |
| | SVASQSIIAYTMSLGAENSVAYSNNSIAIPTNFTISVTTEILPVSMTK | 15) is bolded. |
| | TSVDCTMYICGDSTECSNLLLQYGSFCTQLNRALTGIAVEQDKNTQEV | GCN4 trimer motif is |
| | FAQVKQIYKTPPIKDFGGFNFSQILPDPSKPSKRSFIEDLLFNKVTLA | italicized. |
| | DAGFIKQYGDCLGDIAARDLICAQKFNGLTVLPPLLTDEMIAQYTSAL | |
| | LAGTITSGWTFGAGAALQIPFAMQMAYRFNGIGVTQNVLYENQKLIAN | |
| | QFNSAIGKIQDSLSSTASALGKLQDVVNQNAQALNTLVKQLSSNFGAI | |
| | SSVLNDILSRLDPPEAEVQIDRLITGRLQSLQTYVTQQLIRAAEIRAS | |
| | ANLAATKMSECVLGQSKRVDFCGKGYHLMSFPQSAPHGVVFLHVTYVP | |
| | AQEKNFTTAPAICHDGKAHFPREGVFVSNGTHWFVTQRNFYEPQIITT | |
| | DNTFVSGNCDVVIGIVNNTVYDPLQPELDSFKEELDKYFKNHTSPDVD | |
| | LGDISGINASVVNIQKEIDRLNEVAKNLNESLIDLQELGKYEQYIKGS | |
| | GIKRMKQIEDKIEEIESKQKKIENEIARIKKIKGHHHHHH | |
| 26 | MFVFLVLLPLVSSQCVNLTTRTQLPPAYTNSFTRGVYYPDKVFRSSVL | One embodiment of |
| | HSTQDLFLPFFSNVTWFHAIHVSGTNGTKRFDNPVLPFNDGVYFASTE | engineered SARS-CoV-2 |
| | KSNIIRGWIFGTTLDSKTQSLLIVNNATNVVIKVCEFQFCNDPFLGVY | spike ectodomain (SEO |
| | YHKNNKSWMESEFRVYSSANNCTFEYVSQPFLMDLEGKQGNFKNLREF | ID NO: 6 + Signal |
| | VFKNIDGYFKIYSKHTPINLVRDLPQGFSALEPLVDLPIGINITRFQT | peptide) operably |
| | LLALHRSYLTPGDSSSGWTAGAAAYYVGYLQPRTFLLKYNENGTITDA | linked to a GCN4 |
| | VDCALDPLSETKCTLKSFTVEKGIYQTSNFRVQPTESIVRFPNITNLC | trimer motif, |
| | PFGEVFNATRFASVYAWNRKRISNCVADYSVLYNSASFSTFKCYGVSP | comprising: |
| | TKLNDLCFTNVYADSFVIRGDEVRQIAPGQTGKIADYNYKLPDDFTGC | 1) furin cleavage site |
| | VIAWNSNNLDSKVGGNYNYLYRLFRKSNLKPFERDISTEIYQAGSTPC | mutations (R682A, |
| | NGVEGFNCYFPLQSYGFQPTNGVGYQPYRVVVLSFELLHAPATVCGPK | R683G, R685G), and |
| | KSTNLVKNKCVNFNFNGLTGTGVLTESNKKFLPFQQFGRDIADTTDAV | 2) stabilizing dual |
| | RDPQTLEILDITPCSFGGVSVITPGTNTSNQVAVLYQDVNCTEVPVAI | mutation (K986P, |
| | HADQLTPTWRVYSTGSNVFQTRAGCLIGAEHVNNSYECDIPIGAGICA | V987P). |
| | SYQTQTNSPAGAGSVASQSIIAYTMSLGAENSVAYSNNSIAIPTNFTI | Linker GSG (SEQ ID NO: |
| | SVTTEILPVSMTKTSVDCTMYICGDSTECSNLLLQYGSFCTQLNRALT | 15) is bolded. |
| | GIAVEQDKNTQEVFAQVKQIYKTPPIKDFGGFNFSQILPDPSKPSKRS | GCN4 trimer motif is |
| | FIEDLLFNKVTLADAGFIKQYGDCLGDIAARDLICAQKFNGLTVLPPL | italicized. |
| | LTDEMIAQYTSALLAGTITSGWTFGAGAALQIPFAMQMAYRFNGIGVT | |
| | QNVLYENQKLIANQFNSAIGKIQDSLSSTASALGKLQDVVNQNAQALN | |
| | TLVKQLSSNFGAISSVLNDILSRLDPPEAEVQIDRLITGRLQSLQTYV | |
| | TQQLIRAAEIRASANLAATKMSECVLGQSKRVDFCGKGYHLMSFPQSA | |
| | PHGVVFLHVTYVPAQEKNFTTAPAICHDGKAHFPREGVFVSNGTHWFV | |
| | TQRNFYEPQIITTDNTFVSGNCDVVIGIVNNTVYDPLQPELDSFKEEL | |
| | DKYFKNHTSPDVDLGDISGINASVVNIQKEIDRLNEVAKNLNESLIDL | |
| | QELGKYEQYIKGSGIKRMKQIEDKIEEIESKQKKIENEIARIKKIKGH | |
| | HHHHH | |
| 14 | MFVFLVLLPLVSS | Embodiment of SARS- |
| | | CoV-2 Spike protein |
| | | signal peptide |
| | | (residues 1-13) |
| 15 | GSG | One embodiment of a |
| | | linker |
| 16 | YIPEAPRDGQAYVRKDGEWVLLSTFL | One embodiment of a |
| | | foldon trimerization |
| | | motif |
| 17 | IKRMKQIEDKIEEIESKQKKIENEIARIKKIK | One embodiment of a |
| | | GCN4 trimerization |
| | | motif |
| 18 | GSGYIPEAPRDGQAYVRKDGEWVLLSTFLG | Linker embodiment |
| | | comprising foldon |
| | | trimerization motif |
| 19 | GSGIKRMKQIEDKIEEIESKQKKIENEIARIKKIKG | Linker embodiment |
| | | comprising GCN4 |
| | | trimerization motif |
| 20 | HHHHHH | One embodiment of a |
| | | His tag |
| 21 | ATGTTTGTCTTCCTGGTCCTGCTGCCTCTGGTCTCGTCTCAGTGCGTG | Nucleic acid sequence |
| | AACCTGACTACTAGAACCCAGCTGCCTCCTGCCTATACTAACTCCTTC | encoding the full- |
| | ACCCGCGGCGTGTACTACCCAGACAAGGTGTTCCGCAGCTCCGTGCTG | length spike ptotein |
| | CACTCCACCCAGGATCTGTTCCTGCCCTTCTTCAGCAACGTGACCTGG | |
| | TTCCACGCCATCCACGTGAGCGGCACCAATGGCACCAAGCGGTTCGAC | |
| | AATCCCGTGCTGCCATTCAACGATGGCGTGTACTTCGCCTCCACCGAG | |
| | AAGAGCAACATCATCCGCGGCTGGATCTTCGGCACCACCCTGGACTCC | |
| | AAGACCCAGAGCCTGCTGATCGTGAACAATGCCACCAACGTGGTCATC | |
| | AAGGTGTGCGAGTTCCAGTTCTGCAATGATCCATTCCTGGGCGTGTAC | |
| | TACCACAAGAACAATAAGTCCTGGATGGAGAGCGAGTTCCGCGTGTAC | |
| | AGCTCCGCCAACAATTGCACCTTCGAGTACGTGTCCCAGCCCTTCCTG | |
| | ATGGACCTGGAGGGCAAGCAGGGCAATTTCAAGAACCTGCGCGAGTTC | |
| | GTGTTCAAGAATATCGATGGCTACTTCAAGATCTACTCCAAGCACACC | |
| | CCCATCAACCTGGTGCGCGACCTGCCACAGGGCTTCAGCGCCCTGGAG | |
| | CCACTGGTGGATCTGCCAATCGGCATCAACATCACCAGGTTCCAGACC | |
| | CTGCTGGCCCTGCACCGCAGCTACCTGACCCCAGGCGACAGCTCCAGC | |
| | GGATGGACCGCTGGAGCTGCTGCCTACTACGTGGGCTACCTGCAGCCC | |
| | CGCACCTTCCTGCTGAAGTACAACGAGAATGGCACCATCACCGACGCC | |
| | GTGGATTGCGCCCTGGATCCACTGTCCGAGACAAAGTGCACCCTGAAG | |
| | AGCTTCACCGTGGAGAAGGGCATCTACCAGACCTCCAATTTCCGCGTG | |
| | CAGCCAACCGAGAGCATCGTGCGCTTCCCCAATATCACCAACCTGTGC | |
| | CCATTCGGCGAGGTGTTCAACGCTACCAGGTTCGCCAGCGTGTACGCT | |
| | TGGAATCGCAAGCGCATCTCCAACTGCGTGGCCGACTACAGCGTGCTG | |
| | TACAACTCCGCCAGCTTCTCCACCTTCAAGTGCTACGGCGTGTCCCCC | |
| | ACCAAGCTGAATGATCTGTGCTTCACCAACGTGTACGCCGATAGCTTC | |
| | GTGATCAGGGGCGACGAGGTGCGCCAGATCGCTCCAGGACAGACCGGC | |
| | AAGATCGCTGACTACAATTACAAGCTGCCCGACGATTTCACCGGCTGC | |
| | GTGATCGCCTGGAACTCCAACAATCTGGATAGCAAAGTGGGCGGCAAC | |
| | TACAATTACCTGTACCGCCTGTTCCGCAAGTCCAATCTGAAGCCATTC | |
| | GAGCGCGACATCTCCACCGAGATCTACCAGGCTGGAAGCACCCCATGC | |
| | AATGGAGTGGAGGGCTTCAACTGCTACTTCCCCCTGCAGAGCTACGGC | |
| | TTCCAGCCAACCAACGGAGTGGGATACCAGCCATACAGGGTGGTGGTG | |
| | CTGTCCTTCGAGCTGCTGCACGCTCCAGCTACCGTGTGCGGACCAAAG | |
| | AAGAGCACCAATCTGGTGAAGAACAAGTGCGTGAACTTCAATTTCAAC | |
| | GGCCTGACCGGAACCGGCGTGCTGACCGAGTCCAACAAGAAGTTCCTG | |
| | CCATTCCAGCAGTTCGGAAGGGACATCGCTGATACCACCGACGCCGTG | |
| | CGCGACCCACAGACCCTGGAGATCCTGGATATCACCCCATGCTCCTTC | |
| | GGCGGCGTGAGCGTGATCACCCCAGGAACCAATACCAGCAACCAGGTG | |
| | GCCGTGCTGTACCAGGACGTGAATTGCACCGAGGTGCCAGTGGCTATC | |
| | CACGCTGATCAGCTGACCCCAACCTGGCGCGTGTACAGCACCGGATCC | |
| | AACGTGTTCCAGACCCGCGCCGGATGCCTGATCGGAGCTGAGCACGTG | |
| | AACAATTCCTACGAGTGCGACATCCCAATCGGAGCTGGAATCTGCGCC | |
| | AGCTACCAGACCCAGACCAACTCCCCAAGGAGGGCTCGCAGCGTGGCC | |
| | AGCCAGTCCATCATCGCCTACACCATGTCCCTGGGCGCCGAGAATAGC | |
| | GTGGCCTACAGCAACAATTCCATCGCCATCCCAACCAACTTCACCATC | |
| | TCCGTGACCACCGAGATCCTGCCCGTGTCCATGACCAAGACCAGCGTG | |
| | GACTGCACCATGTACATCTGCGGCGATTCCACCGAGTGCAGCAACCTG | |
| | CTGCTGCAGTACGGCAGCTTCTGCACCCAGCTGAATCGCGCCCTGACC | |
| | GGAATCGCTGTGGAGCAGGATAAGAACACCCAGGAGGTGTTCGCCCAG | |
| | GTGAAGCAGATCTACAAGACCCCCCCAATCAAGGACTTCGGCGGCTTC | |
| | AATTTCAGCCAGATCCTGCCCGATCCAAGCAAGCCCTCCAAGCGCAGC | |
| | TTCATCGAGGACCTGCTGTTCAACAAGGTGACCCTGGCCGATGCCGGC | |
| | TTCATCAAGCAGTACGGCGATTGCCTGGGCGACATCGCTGCCCGCGAC | |
| | CTGATCTGCGCCCAGAAGTTCAATGGCCTGACCGTGCTGCCACCACTG | |
| | CTGACCGATGAGATGATCGCTCAGTACACCTCCGCCCTGCTGGCCGGA | |
| | ACCATCACCAGCGGATGGACCTTCGGCGCTGGAGCCGCCCTGCAGATC | |
| | CCCTTCGCCATGCAGATGGCCTACCGCTTCAACGGCATCGGCGTGACC | |
| | CAGAATGTGCTGTACGAGAACCAGAAGCTGATCGCCAATCAGTTCAAC | |
| | TCCGCCATCGGCAAGATCCAGGACTCCCTGTCCAGCACCGCCAGCGCC | |
| | CTGGGCAAGCTGCAGGATGTGGTGAATCAGAACGCCCAGGCCCTGAAT | |
| | ACCCTGGTGAAGCAGCTGTCCAGCAACTTCGGCGCCATCTCCAGCGTG | |
| | CTGAATGATATCCTGAGCCGCCTGGACAAGGTGGAGGCTGAGGTGCAG | |
| | ATCGATAGGCTGATCACCGGCCGCCTGCAGTCCCTGCAGACCTACGTG | |
| | ACCCAGCAGCTGATCAGGGCTGCTGAGATCAGGGCCAGCGCCAATCTG | |
| | GCTGCTACCAAGATGTCCGAGTGCGTGCTGGGACAGAGCAAGAGGGTG | |
| | GACTTCTGCGGCAAGGGCTACCACCTGATGTCCTTCCCACAGAGCGCC | |
| | CCACACGGAGTGGTGTTCCTGCACGTGACCTACGTGCCAGCCCAGGAG | |
| | AAGAACTTCACCACCGCTCCAGCTATCTGCCACGATGGCAAGGCTCAC | |
| | TTCCCACGCGAGGGCGTGTTCGTGTCCAACGGCACCCACTGGTTCGTG | |
| | ACCCAGCGCAATTTCTACGAGCCCCAGATCATCACCACCGACAATACC | |
| | TTCGTGAGCGGCAACTGCGACGTGGTCATCGGAATCGTGAACAATACC | |
| | GTGTACGATCCCCTGCAGCCAGAGCTGGACTCCTTCAAGGAGGAGCTG | |
| | GATAAGTACTTCAAGAATCACACCAGCCCCGACGTGGATCTGGGCGAC | |
| | ATCTCCGGCATCAATGCCAGCGTGGTGAACATCCAGAAGGAGATCGAC | |
| | CGCCTGAACGAGGTGGCCAAGAATCTGAACGAGTCCCTGATCGATCTG | |
| | CAGGAGCTGGGCAAGTACGAGCAGTACATCAAGTGGCCATGGTACATC | |
| | TGGCTGGGCTTCATCGCCGGCCTGATCGCCATCGTGATGGTGACCATC | |
| | ATGCTGTGCTGCATGACCTCCTGCTGCAGCTGCCTGAAGGGCTGCTGC | |
| | TCCTGCGGCAGCTGCTGCAAGTTCGATGAGGACGATAGCGAGCCCGTG | |
| | CTGAAGGGCGTCAAACTGCACTATACA | |
| 22 | ATGTTTGTCTTCCTGGTCCTGCTGCCTCTGGTCTCGTCTCAGTGCGTG | Nucleic acid sequence |
| | AACCTGACTACTAGAACCCAGCTGCCTCCTGCCTATACTAACTCCTTC | encoding SEQ ID NO: 11 |
| | ACCCGCGGCGTGTACTACCCAGACAAGGTGTTCCGCAGCTCCGTGCTG | |
| | CACTCCACCCAGGATCTGTTCCTGCCCTTCTTCAGCAACGTGACCTGG | |
| | TTCCACGCCATCCACGTGAGCGGCACCAATGGCACCAAGCGGTTCGAC | |
| | AATCCCGTGCTGCCATTCAACGATGGCGTGTACTTCGCCTCCACCGAG | |
| | AAGAGCAACATCATCCGCGGCTGGATCTTCGGCACCACCCTGGACTCC | |
| | AAGACCCAGAGCCTGCTGATCGTGAACAATGCCACCAACGTGGTCATC | |
| | AAGGTGTGCGAGTTCCAGTTCTGCAATGATCCATTCCTGGGCGTGTAC | |
| | TACCACAAGAACAATAAGTCCTGGATGGAGAGCGAGTTCCGCGTGTAC | |
| | AGCTCCGCCAACAATTGCACCTTCGAGTACGTGTCCCAGCCCTTCCTG | |
| | ATGGACCTGGAGGGCAAGCAGGGCAATTTCAAGAACCTGCGCGAGTTC | |
| | GTGTTCAAGAATATCGATGGCTACTTCAAGATCTACTCCAAGCACACC | |
| | CCCATCAACCTGGTGCGCGACCTGCCACAGGGCTTCAGCGCCCTGGAG | |
| | CCACTGGTGGATCTGCCAATCGGCATCAACATCACCAGGTTCCAGACC | |
| | CTGCTGGCCCTGCACCGCAGCTACCTGACCCCAGGCGACAGCTCCAGC | |
| | GGATGGACCGCTGGAGCTGCTGCCTACTACGTGGGCTACCTGCAGCCC | |
| | CGCACCTTCCTGCTGAAGTACAACGAGAATGGCACCATCACCGACGCC | |
| | GTGGATTGCGCCCTGGATCCACTGTCCGAGACAAAGTGCACCCTGAAG | |
| | AGCTTCACCGTGGAGAAGGGCATCTACCAGACCTCCAATTTCCGCGTG | |
| | CAGCCAACCGAGAGCATCGTGCGCTTCCCCAATATCACCAACCTGTGC | |
| | CCATTCGGCGAGGTGTTCAACGCTACCAGGTTCGCCAGCGTGTACGCT | |
| | TGGAATCGCAAGCGCATCTCCAACTGCGTGGCCGACTACAGCGTGCTG | |
| | TACAACTCCGCCAGCTTCTCCACCTTCAAGTGCTACGGCGTGTCCCCC | |
| | ACCAAGCTGAATGATCTGTGCTTCACCAACGTGTACGCCGATAGCTTC | |
| | GTGATCAGGGGCGACGAGGTGCGCCAGATCGCTCCAGGACAGACCGGC | |
| | AAGATCGCTGACTACAATTACAAGCTGCCCGACGATTTCACCGGCTGC | |
| | GTGATCGCCTGGAACTCCAACAATCTGGATAGCAAAGTGGGCGGCAAC | |
| | TACAATTACCTGTACCGCCTGTTCCGCAAGTCCAATCTGAAGCCATTC | |
| | GAGCGCGACATCTCCACCGAGATCTACCAGGCTGGAAGCACCCCATGC | |
| | AATGGAGTGGAGGGCTTCAACTGCTACTTCCCCCTGCAGAGCTACGGC | |
| | TTCCAGCCAACCAACGGAGTGGGATACCAGCCATACAGGGTGGTGGTG | |
| | CTGTCCTTCGAGCTGCTGCACGCTCCAGCTACCGTGTGCGGACCAAAG | |
| | AAGAGCACCAATCTGGTGAAGAACAAGTGCGTGAACTTCAATTTCAAC | |
| | GGCCTGACCGGAACCGGCGTGCTGACCGAGTCCAACAAGAAGTTCCTG | |
| | CCATTCCAGCAGTTCGGAAGGGACATCGCTGATACCACCGACGCCGTG | |
| | CGCGACCCACAGACCCTGGAGATCCTGGATATCACCCCATGCTCCTTC | |
| | GGCGGCGTGAGCGTGATCACCCCAGGAACCAATACCAGCAACCAGGTG | |
| | GCCGTGCTGTACCAGGGCGTGAATTGCACCGAGGTGCCAGTGGCTATC | |
| | CACGCTGATCAGCTGACCCCAACCTGGCGCGTGTACAGCACCGGATCC | |
| | AACGTGTTCCAGACCCGCGCCGGATGCCTGATCGGAGCTGAGCACGTG | |
| | AACAATTCCTACGAGTGCGACATCCCAATCGGAGCTGGAATCTGCGCC | |
| | AGCTACCAGACCCAGACCAACTCCCCAgctggaGCTggcAGCGTGGCC | |
| | AGCCAGTCCATCATCGCCTACACCATGTCCCTGGGCGCCGAGAATAGC | |
| | GTGGCCTACAGCAACAATTCCATCGCCATCCCAACCAACTTCACCATC | |
| | TCCGTGACCACCGAGATCCTGCCCGTGTCCATGACCAAGACCAGCGTG | |
| | GACTGCACCATGTACATCTGCGGCGATTCCACCGAGTGCAGCAACCTG | |
| | CTGCTGCAGTACGGCAGCTTCTGCACCCAGCTGAATCGCGCCCTGACC | |
| | GGAATCGCTGTGGAGCAGGATAAGAACACCCAGGAGGTGTTCGCCCAG | |
| | GTGAAGCAGATCTACAAGACCCCCCCAATCAAGGACTTCGGCGGCTTC | |
| | AATTTCAGCCAGATCCTGCCCGATCCAAGCAAGCCCTCCAAGCGCAGC | |
| | TTCATCGAGGACCTGCTGTTCAACAAGGTGACCCTGGCCGATGCCGGC | |
| | TTCATCAAGCAGTACGGCGATTGCCTGGGCGACATCGCTGCCCGCGAC | |
| | CTGATCTGCGCCCAGAAGTTCAATGGCCTGACCGTGCTGCCACCACTG | |
| | CTGACCGATGAGATGATCGCTCAGTACACCTCCGCCCTGCTGGCCGGA | |
| | ACCATCACCAGCGGATGGACCTTCGGCGCTGGAGCCGCCCTGCAGATC | |
| | CCCTTCGCCATGCAGATGGCCTACCGCTTCAACGGCATCGGCGTGACC | |
| | CAGAATGTGCTGTACGAGAACCAGAAGCTGATCGCCAATCAGTTCAAC | |
| | TCCGCCATCGGCAAGATCCAGGACTCCCTGTCCAGCACCGCCAGCGCC | |
| | CTGGGCAAGCTGCAGGATGTGGTGAATCAGAACGCCCAGGCCCTGAAT | |
| | ACCCTGGTGAAGCAGCTGTCCAGCAACTTCGGCGCCATCTCCAGCGTG | |
| | CTGAATGATATCCTGAGCCGCCTGGACCCACCGGAGGCTGAGGTGCAG | |
| | ATCGATAGGCTGATCACCGGCCGCCTGCAGTCCCTGCAGACCTACGTG | |
| | ACCCAGCAGCTGATCAGGGCTGCTGAGATCAGGGCCAGCGCCAATCTG | |
| | GCTGCTACCAAGATGTCCGAGTGCGTGCTGGGACAGAGCAAGAGGGTG | |
| | GACTTCTGCGGCAAGGGCTACCACCTGATGTCCTTCCCACAGAGCGCC | |
| | CCACACGGAGTGGTGTTCCTGCACGTGACCTACGTGCCAGCCCAGGAG | |
| | AAGAACTTCACCACCGCTCCAGCTATCTGCCACGATGGCAAGGCTCAC | |
| | TTCCCACGCGAGGGCGTGTTCGTGTCCAACGGCACCCACTGGTTCGTG | |
| | ACCCAGCGCAATTTCTACGAGCCCCAGATCATCACCACCGACAATACC | |
| | TTCGTGAGCGGCAACTGCGACGTGGTCATCGGAATCGTGAACAATACC | |
| | GTGTACGATCCCCTGCAGCCAGAGCTGGACTCCTTCAAGGAGGAGCTG | |
| | GATAAGTACTTCAAGAATCACACCAGCCCCGACGTGGATCTGGGCGAC | |
| | ATCTCCGGCATCAATGCCAGCGTGGTGAACATCCAGAAGGAGATCGAC | |
| | CGCCTGAACGAGGTGGCCAAGAATCTGAACGAGTCCCTGATCGATCTG | |
| | CAGGAGCTGGGCAAGTACGAGCAGTACATCAAGGGATCCGGCTACATC | |
| | CCCGAGGCCCCCAGAGACGGCCAGGCCTACGTGAGAAAGGACGGCGAG | |
| | TGGGTGCTGCTGAGCACCTTCCTGGGCCACCACCACCACCACCAC | |
| 23 | ATGTTTGTCTTCCTGGTCCTGCTGCCTCTGGTCTCGTCTCAGTGCGTG | Nucleic acid sequence |
| | AACCTGACTACTAGAACCCAGCTGCCTCCTGCCTATACTAACTCCTTC | encoding SEQ ID NO: 13 |
| | ACCCGCGGCGTGTACTACCCAGACAAGGTGTTCCGCAGCTCCGTGCTG | |
| | CACTCCACCCAGGATCTGTTCCTGCCCTTCTTCAGCAACGTGACCTGG | |
| | TTCCACGCCATCCACGTGAGCGGCACCAATGGCACCAAGCGGTTCGAC | |
| | AATCCCGTGCTGCCATTCAACGATGGCGTGTACTTCGCCTCCACCGAG | |
| | AAGAGCAACATCATCCGCGGCTGGATCTTCGGCACCACCCTGGACTCC | |
| | AAGACCCAGAGCCTGCTGATCGTGAACAATGCCACCAACGTGGTCATC | |
| | AAGGTGTGCGAGTTCCAGTTCTGCAATGATCCATTCCTGGGCGTGTAC | |
| | TACCACAAGAACAATAAGTCCTGGATGGAGAGCGAGTTCCGCGTGTAC | |
| | AGCTCCGCCAACAATTGCACCTTCGAGTACGTGTCCCAGCCCTTCCTG | |
| | ATGGACCTGGAGGGCAAGCAGGGCAATTTCAAGAACCTGCGCGAGTTC | |
| | GTGTTCAAGAATATCGATGGCTACTTCAAGATCTACTCCAAGCACACC | |
| | CCCATCAACCTGGTGCGCGACCTGCCACAGGGCTTCAGCGCCCTGGAG | |
| | CCACTGGTGGATCTGCCAATCGGCATCAACATCACCAGGTTCCAGACC | |
| | CTGCTGGCCCTGCACCGCAGCTACCTGACCCCAGGCGACAGCTCCAGC | |
| | GGATGGACCGCTGGAGCTGCTGCCTACTACGTGGGCTACCTGCAGCCC | |
| | CGCACCTTCCTGCTGAAGTACAACGAGAATGGCACCATCACCGACGCC | |
| | GTGGATTGCGCCCTGGATCCACTGTCCGAGACAAAGTGCACCCTGAAG | |
| | AGCTTCACCGTGGAGAAGGGCATCTACCAGACCTCCAATTTCCGCGTG | |
| | CAGCCAACCGAGAGCATCGTGCGCTTCCCCAATATCACCAACCTGTGC | |
| | CCATTCGGCGAGGTGTTCAACGCTACCAGGTTCGCCAGCGTGTACGCT | |
| | TGGAATCGCAAGCGCATCTCCAACTGCGTGGCCGACTACAGCGTGCTG | |
| | TACAACTCCGCCAGCTTCTCCACCTTCAAGTGCTACGGCGTGTCCCCC | |
| | ACCAAGCTGAATGATCTGTGCTTCACCAACGTGTACGCCGATAGCTTC | |
| | GTGATCAGGGGCGACGAGGTGCGCCAGATCGCTCCAGGACAGACCGGC | |
| | AAGATCGCTGACTACAATTACAAGCTGCCCGACGATTTCACCGGCTGC | |
| | GTGATCGCCTGGAACTCCAACAATCTGGATAGCAAAGTGGGCGGCAAC | |
| | TACAATTACCTGTACCGCCTGTTCCGCAAGTCCAATCTGAAGCCATTC | |
| | GAGCGCGACATCTCCACCGAGATCTACCAGGCTGGAAGCACCCCATGC | |
| | AATGGAGTGGAGGGCTTCAACTGCTACTTCCCCCTGCAGAGCTACGGC | |
| | TTCCAGCCAACCAACGGAGTGGGATACCAGCCATACAGGGTGGTGGTG | |
| | CTGTCCTTCGAGCTGCTGCACGCTCCAGCTACCGTGTGCGGACCAAAG | |
| | AAGAGCACCAATCTGGTGAAGAACAAGTGCGTGAACTTCAATTTCAAC | |
| | GGCCTGACCGGAACCGGCGTGCTGACCGAGTCCAACAAGAAGTTCCTG | |
| | CCATTCCAGCAGTTCGGAAGGGACATCGCTGATACCACCGACGCCGTG | |
| | CGCGACCCACAGACCCTGGAGATCCTGGATATCACCCCATGCTCCTTC | |
| | GGCGGCGTGAGCGTGATCACCCCAGGAACCAATACCAGCAACCAGGTG | |
| | GCCGTGCTGTACCAGGGCGTGAATTGCACCGAGGTGCCAGTGGCTATC | |
| | CACGCTGATCAGCTGACCCCAACCTGGCGCGTGTACAGCACCGGATCC | |
| | AACGTGTTCCAGACCCGCGCCGGATGCCTGATCGGAGCTGAGCACGTG | |
| | AACAATTCCTACGAGTGCGACATCCCAATCGGAGCTGGAATCTGCGCC | |
| | AGCTACCAGACCCAGACCAACTCCCCAgctggaGCTggcAGCGTGGCC | |
| | AGCCAGTCCATCATCGCCTACACCATGTCCCTGGGCGCCGAGAATAGC | |
| | GTGGCCTACAGCAACAATTCCATCGCCATCCCAACCAACTTCACCATC | |
| | TCCGTGACCACCGAGATCCTGCCCGTGTCCATGACCAAGACCAGCGTG | |
| | GACTGCACCATGTACATCTGCGGCGATTCCACCGAGTGCAGCAACCTG | |
| | CTGCTGCAGTACGGCAGCTTCTGCACCCAGCTGAATCGCGCCCTGACC | |
| | GGAATCGCTGTGGAGCAGGATAAGAACACCCAGGAGGTGTTCGCCCAG | |
| | GTGAAGCAGATCTACAAGACCCCCCCAATCAAGGACTTCGGCGGCTTC | |
| | AATTTCAGCCAGATCCTGCCCGATCCAAGCAAGCCCTCCAAGCGCAGC | |
| | TTCATCGAGGACCTGCTGTTCAACAAGGTGACCCTGGCCGATGCCGGC | |
| | TTCATCAAGCAGTACGGCGATTGCCTGGGCGACATCGCTGCCCGCGAC | |
| | CTGATCTGCGCCCAGAAGTTCAATGGCCTGACCGTGCTGCCACCACTG | |
| | CTGACCGATGAGATGATCGCTCAGTACACCTCCGCCCTGCTGGCCGGA | |
| | ACCATCACCAGCGGATGGACCTTCGGCGCTGGAGCCGCCCTGCAGATC | |
| | CCCTTCGCCATGCAGATGGCCTACCGCTTCAACGGCATCGGCGTGACC | |
| | CAGAATGTGCTGTACGAGAACCAGAAGCTGATCGCCAATCAGTTCAAC | |
| | TCCGCCATCGGCAAGATCCAGGACTCCCTGTCCAGCACCGCCAGCGCC | |
| | CTGGGCAAGCTGCAGGATGTGGTGAATCAGAACGCCCAGGCCCTGAAT | |
| | ACCCTGGTGAAGCAGCTGTCCAGCAACTTCGGCGCCATCTCCAGCGTG | |
| | CTGAATGATATCCTGAGCCGCCTGGACCCACCGGAGGCTGAGGTGCAG | |
| | ATCGATAGGCTGATCACCGGCCGCCTGCAGTCCCTGCAGACCTACGTG | |
| | ACCCAGCAGCTGATCAGGGCTGCTGAGATCAGGGCCAGCGCCAATCTG | |
| | GCTGCTACCAAGATGTCCGAGTGCGTGCTGGGACAGAGCAAGAGGGTG | |
| | GACTTCTGCGGCAAGGGCTACCACCTGATGTCCTTCCCACAGAGCGCC | |
| | CCACACGGAGTGGTGTTCCTGCACGTGACCTACGTGCCAGCCCAGGAG | |
| | AAGAACTTCACCACCGCTCCAGCTATCTGCCACGATGGCAAGGCTCAC | |
| | TTCCCACGCGAGGGCGTGTTCGTGTCCAACGGCACCCACTGGTTCGTG | |
| | ACCCAGCGCAATTTCTACGAGCCCCAGATCATCACCACCGACAATACC | |
| | TTCGTGAGCGGCAACTGCGACGTGGTCATCGGAATCGTGAACAATACC | |
| | GTGTACGATCCCCTGCAGCCAGAGCTGGACTCCTTCAAGGAGGAGCTG | |
| | GATAAGTACTTCAAGAATCACACCAGCCCCGACGTGGATCTGGGCGAC | |
| | ATCTCCGGCATCAATGCCAGCGTGGTGAACATCCAGAAGGAGATCGAC | |
| | CGCCTGAACGAGGTGGCCAAGAATCTGAACGAGTCCCTGATCGATCTG | |
| | CAGGAGCTGGGCAAGTACGAGCAGTACATCAAGGGATCCGGCATAAAA | |
| | AGGATGAAGCAAATCGAGGATAAAATTGAGGAAATCGAGTCGAAGCAA | |
| | AAAAAAATCGAGAATGAGATAGCGCGAATCAAGAAGATAAAG | |
| | GGCCACCACCACCACCACCAC | |
| 24 | ELGKYEQYIKWPWYIWLGFI | Residues 1,202-1,221, |
| | | in reference to SEQ ID |
| | | NO: 1. |

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed is:

1. A method for detecting an anti-SARS-CoV-2 antibody in a sample, the method comprising 1) contacting the sample with a polypeptide comprising a SARS-CoV-2 spike ectodomain amino acid sequence comprising a D614G mutation, under conditions suitable for an anti-SARS-CoV-2 antibody to bind to the polypeptide; and 2) detecting the presence of an anti-SARS-CoV-2 antibody bound to the polypeptide.

2. The method of claim 1, wherein anti-SARS-CoV-2 antibody titers are measured.

3. The method of claim 1, wherein the sample is from an animal and wherein the method further comprises diagnosing the animal as having or having had a SARS-CoV2 infection when anti-SARS-CoV-2 antibodies are detected, and optionally, further comprises administering an anti-SARS-CoV-2 therapeutic agent to the animal.

4. The method of claim 1, wherein the polypeptide is resistant to furin cleavage.

5. The method of claim 4, wherein the polypeptide further comprises R682A, R683G and R685G mutations.

6. The method of claim 1, wherein the polypeptide further comprises K986P and V987P mutations.

7. The method of claim 1, wherein the spike ectodomain amino acid sequence comprises an amino acid sequence having at least about 80%, 85%, 90%, or 95% sequence identity to SEQ ID NO:3, and wherein the spike ectodomain amino acid sequence is further linked to 1 to 20 (e.g., consecutive) amino acids provided in a sequence corresponding to SEQ ID NO:24.

8. The method of claim 1, wherein the spike ectodomain amino acid sequence comprises an amino acid sequence having at least about 80%, 85%, 90%, or 95% sequence identity to any one of SEQ ID NOs:4-7.

9. The method of claim 8, wherein the spike ectodomain amino acid sequence comprises an amino acid sequence having at least about 95% sequence identity to SEQ ID NO:4.

10. The method of claim 8, wherein the spike ectodomain amino acid sequence comprises SEQ ID NO:7.

11. The method of claim 1, wherein the polypeptide further comprises a trimerization motif, and wherein the trimerization motif is operably linked to the spike ectodomain amino acid sequence.

12. The method of claim 11, wherein the trimerization motif is a foldon trimer motif or a GCN4 motif.

13. The method of claim 12, wherein the trimerization motif is a foldon trimer motif comprising SEQ ID NO:16 or a GCN4 motif comprising SEQ ID NO:17.

14. The method of claim 1, wherein the polypeptide is further operably linked to a detectable marker.

15. The method of claim 1, wherein the polypeptide comprises an amino acid sequence having at least about 80%, 85%, 90%, or 95% sequence identity to SEQ ID NO:10, 11, 12 or 13.

16. The method of claim 15, wherein the polypeptide comprises SEQ ID NO:10.

17. The method of claim 15, wherein the polypeptide comprises SEQ ID NO:12.

18. The method of claim 11, wherein the polypeptide is trimerized.

19. The method of claim 1, wherein the polypeptide is immobilized on a substrate.

20. A polypeptide comprising a SARS-CoV-2 spike ectodomain amino acid sequence comprising D614G, R682A, R683G, R685G, K986P and V987P mutations.
